(19) United States
(12) Patent Application Publication
(10) Pub. No.: US 2024/0138459 A1
(43) Pub. Date: May 2, 2024

(54) NUTRITIONAL FORMULATIONS COMPRISING A PEA PROTEIN ISOLATE

(71) Applicant: ROQUETTE FRERES, Lestrem (FR)

(72) Inventors: Manuel Barata, Maisnil Les Ruitz (FR); Marilyne Guillemant, Aire sur la Lys (FR); Emmanuelle Moretti, Lille (FR); Elsa Müller, Bethune (FR); Marie Delebarre, La Couture (FR)

(21) Appl. No.: 18/380,818

(22) Filed: Oct. 17, 2023

Related U.S. Application Data
(63) Continuation of application No. 16/070,311, filed on Jul. 16, 2018, now abandoned.

(30) Foreign Application Priority Data
Jan. 29, 2016 (FR) 1650710
Mar. 7, 2016 (FR) 1651865
Apr. 29, 2016 (FR) 1653861
Jul. 8, 2016 (FR) 1656605

(51) Int. Cl. A23L 33/185; A23J 3/14; A23J 3/34; A23L 2/66; A23L 11/60; A23L 33/00; A23L 33/19

(52) U.S. Cl. CPC A23L 33/185; A23J 3/14; A23J 3/346; A23L 2/66; A23L 11/60; A23L 33/19; A23L 33/40; A23C 9/13

(57) ABSTRACT

The present invention relates to a nutritional formulation containing a pea protein isolate, characterized in that the pea protein isolate:
contains between 0.5 and 2% of free amino acids,
has a viscosity:
   from 13 to 16×10⁻³ Pa·s. at a shear rate of 10 s⁻¹,
   from 10 to 14×10⁻³ Pa·s. at shear rate of 40 s⁻¹, and
   from 9.8 to 14×10⁻³ Pa·s. at a shear rate of 600 s⁻¹,
has a solubility:
   from 30 to 40% in pH zones from 4 to 5
   from 40 to 70% in pH zones from 6 to 8.

The invention also relates to the use of this nutritional formulation as a single protein source or as a food supplement, intended for infants, children and/or adults.

### TECHNICAL FIELD OF THE INVENTION

The present invention relates to nutritional formulations comprising a pea protein isolate.

More particularly, the invention relates to the application of these nutritional formulations:
as beverages, by means of powder mixtures to be reconstituted, especially for dietetic nutrition (sport, slimming),
as ready-to-drink beverages for dietetic or clinical nutrition,
as liquids (enteral bags or beverages) for clinical nutrition,
as fermented milks of yoghurt type (stirred, Greek, drinking, etc. yoghurt),
as dairy/plant-based beverages,
as dairy/plant-based creams (such as coffee cream or “coffee whitener”), dessert creams, iced desserts or sorbets,
as biscuits, muffins, griddle cakes or nutritional bars (intended for specialized/slimming or sports nutrition),
as protein-enriched breads or gluten-free breads,
as high-protein cereals, obtained by extrusion cooking (“crisps” for inclusion/breakfast cereals/snacks).
as cheeses.

### CONTEXT OF THE INVENTION

Nutritional Powders and Liquids

The nutritional powders and liquids manufactured in pediatrics, for infants or adults comprise a well-defined selection of nutritional ingredients (carbohydrates, protein, fat, fiber, vitamins and/or trace elements, etc.).

Some are used as a single food source, while others are used as food supplements.

These nutritional products comprise powders which may be reconstituted, with water or another aqueous liquid, as nutritional liquids such as enteral bags or ready-to-drink beverages.

These nutritional formulations in powder and liquid form for ready-to-drink beverages and enteral bags are particularly popular in nutrition and their use is on the increase worldwide.

Nutritional formulations in powder form are typically prepared by intimately mixing various powders.

Ready-to-drink or enterally-administered nutritional formulations are typically prepared by making one or two separate solutions which are then mixed together, and then heat-treated to allow conservation for at least 12 months at room temperature.

A first solution represents the aqueous phase containing carbohydrates, protein, fiber, minerals and water-soluble emulsifiers, and the second represents the lipid phase containing the oil and liposoluble emulsifiers.

It is well known that the addition of this second lipid phase depends on the nutritional formulations targeted.

These nutritional formulations in powder and liquid form are especially sought for their supply of protein and their supply of energetic nutrients.

Conventionally, use has been made above all of milk protein.

However, for reasons of cost and environmental considerations, it is preferred to make use of plant protein as an alternative to milk protein for protein enrichment in powder mix beverages and ready-to-drink beverages.

Soybean protein (isolates, hydrolyzates) is used in the vast majority, but also rice, wheat and potato protein (especially for improving the vegetable taste of finished products).

In the context of the revegetation of market products and of cost reduction, it may be proposed to develop novel solutions based on pea protein as an alternative to milk protein for protein enrichment, in finished products such as beverages (powder mix to be reconstituted for dietetic nutrition (sport/slimming) and ready-to-drink beverages for clinical and dietetic nutrition), and enteral bags.

In this case, the pea protein must satisfy certain functionalities such as good solubility, low viscosity in solution, good resistance to heat treatments for the heat-treated liquids, and also good viscosity stability over time.

It must also satisfy the nutritional recommendations recommended by the FAO/WHO, in terms of amino acid profile and digestibility profile.

Now, it has been found that when it is chosen to use protein extracted from pea as a dry mix in nutritional powder bases, even at very low concentrations, and when an attempt is made to reconstitute the nutritional formulation, said formulation may have an undesirable sandy feel in the mouth, associated with the granulometry, the solubility and the composition of said protein.

The excessive viscosity of formulations with a high protein content containing pea protein is also a source of dissatisfaction.

An alternative solution to milk protein must thus imperatively comply with the good sensory and functional properties which milk protein naturally satisfies.

Fermented Milks of Desserts Such as Stirred, Greek and Set Yoghurts

A yoghurt is a milk seeded with lactic acid ferments in order to thicken it and to conserve it for longer.

In order to be called a yoghurt, it must necessarily, and only, contain two specific ferments, Lactobacillus delbrueckii subsp. bulgaricus and Streptococcus thermophilus, which give it its taste specificity and its texture, and also provide certain nutritional and health benefits.

Other fermented milks (with a yoghurt texture) have been created in recent years. They may or may not contain these two bacteria, and in addition strains such as Lactobacillus acidophilus, Lactobacillus casei, Bifidobacterium bifidum, B. longum, B. infantis and B. breve.

Yoghurts are thus an excellent source of probiotics, i.e. of live microorganisms, which, when ingested in sufficient amount, exert positive health effects, beyond the conventional nutritional effects.

Whether it is set, stirred or liquid, the name yoghurt is retained since, in point of fact, beyond the regulatory definitions, it is its manufacture which conditions its final texture.

Thus, to obtain a set yoghurt, the milk is seeded directly in the pot.

On the other hand, in the case of a stirred yoghurt (also called “bulgarian” yoghurt), the milk is seeded in a tank and then stirred, before being poured into its pot.

Finally, liquid yoghurt, also called drinking yoghurt, is stirred and then blended until the appropriate texture is obtained, and is poured into bottles.

However, other types of plain yoghurt also exist, such as Greek yoghurt, which has a thicker texture.

The percentage of fat may also modify the texture of the yoghurt, which may be manufactured based on whole milk, semi-skimmed milk or skimmed milk (a label comprising only the word “yoghurt” necessarily denotes a yoghurt made with semi-skimmed milk).

In all cases, its expiry date cannot exceed 30 days and it must always be stored in a refrigerator between 0° and 6°.

Three main classes of yoghurt are distinguished as follows:

Stirred Yoghurt

More liquid, it is often more acidic than plain yoghurt. Only its texture differs. It is also known as bulgarian yoghurt—in reference to the supposed origins of yoghurt and to Lactobacillus bulgaricus, one of the two ferments involved in the transformation of milk into yoghurt. It is manufactured in a tank before being packaged in pots.

It is particularly suitable for making beverages, such as lassis, fruit cocktails, etc.

Greek Yoghurt

This particularly thick yoghurt is a plain yoghurt that has been considerably strained (traditional technique) or enriched with cream. This very tasty, gourmet yoghurt is essential for the preparation of tsatsiki and for Eastern European dishes, and quite simply mixed with fines herbes, it is a delicious aperitif dip. Used cold, it can be used as a replacement for thick crème fraîche.

Drinking Yoghurt

Although it exists in plain form, it is usually sweetened and flavored, and manufactured with a blended stirred yoghurt. Conceived of in 1974, it has enabled adolescents to rediscover the pleasure of milk, by eating yoghurt without a spoon, direct from the bottle. “Pouring yoghurt”, in a 950 g carton, has also recently come into existence, for those who wish to combine cereals and yoghurt for breakfast.

This low energy—52 kcal for a fat-free yoghurt made from skimmed milk, 88 kcal for a whole milk yoghurt—“plain” yoghurt is naturally low in fat and carbohydrates, but contains a fair amount of protein. It is also a source of micronutrients (especially calcium and phosphorus), as well as vitamins B2, B5, B12 and A. Yoghurt, which is constituted of 80% water, participates actively in hydrating the body.

Regular consumption of yoghurt is thus acknowledged to improve the digestion and absorption of lactose (EFSA opinion of Oct. 19, 2010). Other studies show potential benefits on improving diarrhea in children and on the immune system in certain persons such as the elderly.

However, the consumption of cow's milk is subject to increasing criticism and questioning, and an increasing number of people are quite simply deciding to cut it out of their diet, for example for reasons of lactose intolerance, or for allergenicity problems.

Plant-milk-based yoghurt solutions have thus been proposed, since plant milks are much easier to digest than cow's milk, and are rich in vitamins, minerals and unsaturated fatty acids.

In the rest of the present description, for the sake of simplicity, the term “yoghurt” will continue to be used, even if the origin of the protein is not dairy (officially, “yoghurts” that are manufactured from ingredients other than fermented milk, dairy ingredients or conventional ferments such as Lactobacillus delbrueckii subsp bulgaricus and Streptococcus thermophilus do not have the right to be named as such).

The plant source most commonly used is soybean. However, although soybean milk has the highest richness in calcium and protein, it is also very indigestible; this is why it is not recommended for children.

Furthermore, it is not recommended either to excessively consume soybean-based products since their effects on health may be counter productive when they are consumed in large amount.

Moreover, it is commonly accepted that 70% of the worldwide production of soybean is from GMO sources.

Milk and Dairy Beverages; Plant-Based Beverages

Milk is a food which contains a not insignificant protein source of high biological quality. For a long time, animal proteins were overwhelmingly favored for their excellent nutritional qualities, since they contain all the essential amino acids in adequate proportions.

However, certain, animal proteins may be allergenic, entailing reactions that are particularly troublesome, or even hazardous in dally life.

Dairy product allergy is one of the most widespread allergic reactions. Studies demonstrate that 65% of people who suffer from food allergies are allergic to milk. The adult form of milk allergy, referred to herein as “dairy product allergy”, is a reaction of the immune system which creates antibodies in order to combat the undesirable food. This allergy is different from cow's milk protein (bovine protein) allergy, also referred to as CMPA, which affects infants and infants. The clinical manifestations of this allergy are mainly gastrointestinal (50 to 80% of cases), and also cutaneous (10 to 39% of cases) and respiratory (19% of cases).

In the light of all the drawbacks mentioned above associated with the consumption of dairy protein, there is great interest in the use of replacement proteins, also known as alternative proteins, among which are plant proteins.

Plant-based milks, obtained from plant ingredients, may be an alternative to milks of animal origin. They overcome and avoid CMPA. They are free of casein, lactose and cholesterol, are rich in vitamins and mineral salts, and are also rich in essential fatty acids, but low in saturated fatty acids. Some also have a fair content of fiber.

Besides the fact that certain plant-based milks are low in calcium, and that others are commercially unavailable on account of their botanical rarity, it should also be mentioned that certain plant-based milks are also allergenic. This is the case, for example, for plant-based milks prepared from oleaginous plants, for instance soybean milks.

In the light of all the drawbacks of dairy protein, but also of the hazardous allergenic nature imparted by certain plant proteins, there is a real demand from consumers, who are unsatisfied to date, for plant-based milks which have indisputable and acknowledged harmlessness and which as a result can be consumed by all the family. Conventional manufacturers are also commencing the search for novel protein sources to enrich their products.

The Applicant Company has also addressed this search in order to be able to meet the increasing demands of manufacturers and consumers for compositions which have advantageous nutritional properties, without however having the drawbacks of certain already-existing compounds. The Applicant's studies have related to the formulation of novel plant-based milks which have indisputable and acknowledged harmlessness and which as a result can be consumed by all the family.

Dairy Creams for Coffee Cream, Butter, Cheese, Chantilly Creams, Sauces, Cake Toppings and Decorations

Dairy creams are products containing more than 30% fat, obtained by concentrating milk, and are in the form of an emulsion of oil droplets in skimmed milk. They may be used for various applications, either directly as a consumer product (for example used as a coffee cream) or as an industrial raw material for the manufacture of other products such as butter, cheese, chantilly creams, sauces, ice creams, or alternatively cake toppings and decorations.

Various varieties of creams exist: crème fraîche, low-fat cream, single cream, double cream, pasteurized cream. Creams differ according to their fat content, their conservation and their texture.

Raw cream is cream obtained from the separation of milk and cream, directly after skimming and without performing a pasteurization step. It is liquid and contains from 30 to 40% fat.

Pasteurized cream, which is still of liquid texture, has undergone the pasteurization process. It has thus been heated at 72° C. for about 20 seconds so as to remove the microorganisms that are harmful to humans. This cream is particularly suitable for expanding. It thus takes on a lighter and more voluminous texture on being whipped to incorporate air bubbles therein. It is perfect for chantilly creams, for example.

Certain fluid creams sold in shops are termed as being “long-life”. They may be stored for several weeks in a cool, dry place. To be conserved for such a long time, these creams have either been sterilized, or heated via the UHT process. For sterilization, it is a matter of healing the cream for 15 to 20 minutes at 115° C. With the UHT (or Ultra-High Temperature) process, the cream is heated for 2 seconds at 150° C. The cream is then rapidly cooled, the result of which is that its taste qualities are better conserved.

Cream is naturally fluid, once it has been separated from milk, after skimming. In order for it to take a thick texture, it passes through the seeding step. Lactic acid ferments are thus incorporated and, after maturation, give the cream a thicker texture and a more acidic and richer taste.

Along with the conventional techniques (dating back millennia or centuries) for obtaining cream from milk, techniques for assembling or reconstituting cream from dairy ingredients have been developed in the last decade.

These novel techniques for reconstituting dairy creams have obvious advantages in industrial processes, compared with crème fraîche: low cost of storage of the raw materials, greater formulation flexibility, independence from the seasonal effect on the composition of the milk.

Thus, reconstituted dairy creams can benefit from the natural image generally attributed to dairy products, since the regulations stipulate for their manufacture the exclusive use of dairy ingredients with or without addition of drinking water and the same finished product characteristics as milk cream (Codex Alimentarius, 2007).

The development of the field of reconstituted dairy creams has opened up new possibilities in the formulation of creams, and more particularly that of the birth of the concept of plant-based creams.

Plant-based creams are products that are similar to dairy creams, the dairy fat of which is replaced with plant fat (Codex Alimentarius, codex Stan 192, 1995).

They are formulated starting with well-defined amounts of water, plant fat, dairy or plant protein, stabilizers, thickeners and emulsifiers of low molecular weight.

The physicochemical parameters, such as the particle size, the rheology, the stability and the expandability, are the characteristics which are of chief interest to manufacturers and researchers in the field of substitution of dairy creams with plant-based creams.

For example, as in any emulsion, the size of the dispersed droplets (particle size) is a key parameter in the characterization of creams since it has an appreciable impact firstly on the other physicochemical properties such as the meology and the stability, and secondly on the sensory properties such as the texture and color of creams.

The influence of the type of emulsifier Includes both low molecular weight emulsifiers such as monoglycerides, diglycerides and phospholipids, and high molecular weight emulsifiers such as proteins, and also protein/low molecular weight emulsifier interactions.

It is thus known that the concentration of the lipid emulsifier also has an influence on the droplet size of creams. In protein-stabilized systems, a very high concentration of the lipid emulsifier can cause a high increase in the mean droplet size, due to substantial aggregation of the droplets following desorption of the proteins.

The type of protein used in the formulation may also affect the particle size of creams. Specifically, under the same emulsification conditions, creams based on casein-rich protein sources, such as skimmed milk powder generally have smaller mean droplet diameters than those based on whey protein-rich protein sources, such as whey powder.

The particle size differences between creams prepared from the two protein sources (caseins of whey proteins) are linked to the differences in interfacial properties at the oil/water interface, caseins having a higher capacity to lower the interfacial tension than whey proteins.

Moreover, the protein concentration in the formulation has an influence on the particle size of creams. Specifically, it has been demonstrated that for a constant mass fraction of oil, the droplet size decreases as the protein concentration increases, up to a certain concentration beyond which the size varies very little.

The simultaneous presence of amphiphilic molecules of low molecular weight (surfactant) and high molecular weight (proteins) in a cream formulation is generally reflected by a decrease in the droplet size during emulsification. Moreover, competitive adsorption at the oil/water interface between surfactants and proteins generally leads during maturation to desorption of the proteins at the surface of the droplets, which may entail particle size changes.

Finally, it appears that the emulsification conditions, the choice of the ingredients (both proteins and lipids) used in the formulation, and the temperature, have an influence on the final properties of creams.

It appears that plant-based creams may lead to novel techno-functional properties. Thus, the resistance to freezing, which may impart great stability on Ice creams, is an example thereof. They may also show cook-and-serve or cook-and-chill stability, which is a considerable advantage, since these creams may be used either in the preparation of hot or cold meals.

While plant-based creams may afford novel functionalities and show textural properties comparable to or even more interesting than those of dairy creams, it nevertheless remains that they may have sensory defects, especially with regard to their taste and their odor, even sometimes after the addition of flavorings (which is the case for soybean protein or pea protein).

The Applicant Company thus conducted studies on plant-based creams (including the field of “non-dairy” coffee creamers) so as to further the understanding regarding the influence of their Ingredients, such as pea protein, and their interactions with each other (protein-protein, protein-fat, protein-water, etc.) on the final properties of the creams.

The Applicant Company also developed vegan cheese recipes.

Cheese is normally a food obtained from coagulated milk or from dairy cream, followed by straining and then optionally fermentation and optional maturing.

Cheese is thus manufactured mainly from cow's milk, but also from the milk of goats, sheep, buffaloes or other mammals. The milk is acidified, generally using a bacterial culture. An enzyme, rennet, or a substitute such as acetic acid or vinegar, is then added so as to bring about coagulation and to form clotted milk and whey.

It is known practice to prepare vegan alternatives to cheese (especially mozzarella-type cheeses) by replacing milk caseinates with native and modified starches, more particularly acetate-stabilized starches.

However, it is still sought to improve the shreddability, the melting, the stability to freezing/thawing and the taste (especially in the United States for pizza preparations).

Tests were conducted combining oil, modified starches and pea protein, but were not entirely satisfactory.

The Applicant Company found that the use of the pea protein isolates in accordance with the invention made it possible to satisfy these specifications, especially in terms of shreddability, melting and taste.

Ice Creams

Ice creams conventionally contain animal or plant fats, protein (milk protein, egg protein) and/or lactose.

The protein then acts as texturizer in addition to giving the ice cream taste.

They are essentially produced by weighing out the ingredients, premixing them, homogenizing, pasteurizing and refrigerating them at 4° C. (allowing maturation), followed by freezing before packaging and storing.

However, many people suffer from intolerance to dairy products or other ingredients of animal origin, which prevent them from consuming milk or conventional ice cream.

For this group of consumers, there has hitherto been no alternative to ice cream containing milk which has comparable sensory value.

In the ice cream preparations known hitherto containing plant ingredients, mainly based on soybean, attempts were made to replace the animal emulsifiers with plant proteins.

Dried plant proteins, obtained in conventional aqueous or aqueous-alcoholic extraction processes and in powder form after drying, were often used.

These proteins prove to be heterogeneous mixtures of polypeptides, certain fractions of which have variable degrees of particularly good properties such as emulsifiers or gel-forming agents, as water binding agents, foaming agents or texture-improving agents.

Hitherto, plant protein products were obtained almost exclusively from soybean, without fractionation as a function of their specific functional properties.

Moreover, the taste of ice creams prepared with said soybean protein is offputting.

The Applicant Company thus conducted studies on plant-based creams and found that the pea protein isolates according to the invention, made it possible to satisfy the desired specifications.

High Protein Biscuit Products, Pastry Products, Breadmaking Products and Cereal Products

To obtain the “protein-rich” designation, it is necessary, according to the regulations in force, for the calorific supply associated with the proteins to be greater than or equal to 20% of the total energy supply of the finished product.

This means that, in products with an appreciable fat content such as biscuits or cakes (between 10% for the leanest to 25% for the richest with an average of 18% fat), the degree of protein incorporation to achieve the designation is substantial and is greater than 20%.

However, replacing at least one fifth of the formulation with a protein, irrespective of the protein and irrespective of the matrix (biscuits/cake) is a real technological challenge, since these reformulations are not without consequence on:
the structural and/or textural properties of the high-protein preparations thus produced (associated especially with their level of hydration) and also of the finished products,
the process for manufacturing the high-protein preparations (ability to be fashioned by molding, “machinability”),
the sensory quality of the high-protein preparations and of the finished products.

The Applicant Company has already proposed a pes protein, NUTRALYS® BF, for increasing the protein content of biscuits, while limiting the negative impacts on the preparation and the finished product.

The solution came from a pea protein having little or no functional properties (emulsifying power/gelling power) and little interaction with water, this protein being sparingly soluble.

However, this protein does not make it possible to fully satisfy the technical problems mentioned above.

Thus, it is possible to obtain good results on “protein source” biscuits, i.e. biscuits in which 12% of the total calorific supply is provided by the proteins.

However, on “protein-rich” designations, this protein NUTRALYS® BF has limits and the products are not optimized in terms of texture, this texture remaining pasty.

The Applicant Company thus continued working to optimize the qualities of plant proteins, especially derived from peas, by proposing novel pea protein isolates in accordance with the invention, which better satisfy the technological challenges such as the protein enrichment of baking products.

Specifically, the pea protein isolate obtained according to the invention makes it possible to combine the benefits of NUTRALYS® BF, namely little functionality (emulsifier power/gelling power) but with high solubility.

The Applicant Company has thus found that these two properties, which to its knowledge have never been combined to date, could be combined to offer a protein source allowing a high protein enrichment without a negative impact on the preparation process or texture of the preparations or finished products.

### SUMMARY OF THE INVENTION

The present invention proposes novel nutritional formulations containing a pea protein isolate that can totally or partly substitute for milk or soybean protein, of neutral taste, and which have properties suitable for:
mixing powders,
ready-to-drink UHT sterilized beverages containing protein (or even protein-rich or “high-protein”) and
enterally administered nutritional liquids,

in which low viscosity of the beverage and Improvement of the pea protein solubility are desired and also in:
fermented milks of yoghurt type (stirred, Greek, drinking, etc. yoghurt),
dairy/plant-based beverages,
dairy/plant-based creams (such as “coffee whitener”), iced desserts or sorbets,

in which the emulsifying capacity of said pea protein isolate is of interest for its use in the matrices of these dairy products in partial or total substitution for dairy protein,
vegan cheeses,

in which the addition of said pea protein isolate makes it possible to improve the shreddability, the melting and the taste of mozzarella-type vegan cheeses.

The present invention also proposes novel nutritional formulations containing a pea protein isolate having properties suitable for:
biscuits, muffins, griddle cakes of nutritional bars (intended for specialized/slimming or sports nutrition),
protein-enriched breads or gluten-free breads,
high-protein cereals, obtained by extrusion cooking (“crisps” for inclusion/breakfast cereals/snacks).

The invention also leads to improving the taste of the pea protein (reducing the pea notes, green notes) in order to be more neutral in the applications/finished products (with a high content of protein and standard) using the pea protein isolate in partial or total substitution for milk, protein, which is an important property for all types of dairy products, dairy or plant-based beverages, fermented milks of yoghurt type, dairy or plant-based creams, etc.

The subject of the invention is, precisely, a nutritional formulation comprising a pea protein isolate which:
contains between 0.5 and 2% of free amino acids,
has a viscosity at 20° C.:
   from 11 to 18×10⁻³ Pa·s. at a shear rate of 10 s⁻¹,
   from 9 to 16×10⁻³ Pa·s. at a shear rate of 40 s⁻¹, and
   from 8 to 16×10⁻³ Pa·s. at a shear rate of 600 s⁻¹,
has a solubility:
   from 30 to 40% in pH zones from 4 to 5
   from 40 to 70% in pH zones from 6 to 8.

Preferably, the pea protein, isolate has a digestibility expressed according to the Coefficient of Digestive Use (CDU) of between 93.5 and 95%.

Preferably, the pea protein isolate has a degree of hydrolysis (DH) of between 5 and 10%.

In particular, the pea protein isolate is presented, according to the SYMPHID test, as a protein of “rapid viscosity”, reflecting rapid duodenal assimilation of the constituent amino acids of said isolate.

Preferably, the pea protein isolate has been pasteurized at high temperature for a short time before being dried by atomization.

In one embodiment of the present invention, the nutritional formulation comprises at least one pea protein isolate and at least one milk protein. The milk protein preferably represents at least 10, 15, 20, 25, 30, 40, 45 or 50% by weight relative to the total weight of proteins, when the nutritional formulation is in powder form.

In another embodiment of the present invention, the nutritional formulation comprises at least one pes protein isolate, another plant protein, such as a soybean, rice and/or wheat protein, and at least one milk protein.

The pea protein isolate represents:
between 40 and 100%, preferably between 50 and 100%, 60-100%, 70-100%, 80-100% or 50-90% of the total protein in the nutritional formulation in powder form.
between 0.1% and 100% of the total protein for ready-to-drink beverages for clinical nutrition and slimming, preferably between 20-100%, 30-100%, 40-100%, 50 and 100%, 60-100%, 70-100%, 80-100% or 50-90% of the total protein in the nutritional formulation and
between 52% and 100% of the total protein for ready-to-drink beverages for sports nutrition, 60-100%, 70-100%, 80-100% or 50-90% of the total protein in the nutritional formulation,
between 0.1% and 100% of the total protein for fermented milks of yoghurt type, preferably between 20-100%, 30-100%, 40-100%, 50 and 100%, 60-100%, 70-100%, 80-100%, 20-60%, 30-50% or 50-90% of the total protein in the nutritional formulation,
between 0.1% and 100% of the total protein for dairy beverages, preferably between 20-100%, 30-100%, 40-100%, 50 and 100%, 60-100%, 70-100%, 80-100% or 50-90% of the total protein in the nutritional formulation,
between 0.1% and 100% of the total protein for dairy creams, iced desserts of sorbets, more particularly between 50-100%, 60-100%, 70-100%, 80-100% or 50-90% of the total protein for coffee whitener and between 20-100%, 30-100%, 40-100%, 50-100%, or 40-90% of the total protein for dairy creams, iced desserts or sorbets,
between 5% and 100% of the total protein for biscuits, muffins, griddle cakes of nutritional bars (intended for specialized/slimming nutrition or sports nutrition), preferably between 20-100%, 30-100%, 40-100%, 50 and 100%, 60-100%, 70-100%, 80-100% or 50-90% of the total protein in the nutritional formulation,
between 5% and 100% of the total protein for protein-enriched breads or gluten-free breads, preferably between 10-100%, 20-100%, 30-100%, 40-100%, 50 and 100%, 60-100%, 70-100%, 80-100% or 50-90% of the total protein in the nutritional formulation,
between 5% and 100% of the total protein for high-protein cereals, obtained by extrusion cooking (“crisps” for inclusion/breakfast cereals/snacks), preferably between 20-100%, 30-100%, 40-100%, 50 and 100%, 60-100%, 70-100%, 80-100% or 50-90% of the total protein in the nutritional formulation.

For vegan cheeses, about 5% by weight of pea protein isolate in the recipe is sufficient to improve their technical and organoleptic characteristics.

For example, the pea protein isolate according to the present invention may represent 0.1-10%, 10-20%, 20-30%, 40-50%, 50-60%, 60-70%, 70-80%, 80-90% or 90-100%, in particular by weight, of the total protein in the nutritional formulation, or any combination of these percentage ranges.

A subject of the invention is also a nutritional formulation as described above, for use as a single protein source or as a food Supplement, intended for infants, children and/or adults.

A subject of the invention is also the use of this nutritional formulation as a single protein source or as a food supplement, intended for Infants, children and/or adults.

### DETAILED DESCRIPTION OF THE INVENTION

The present invention relates to nutritional formulations comprising a pea protein isolate according to the present invention. The invention also relates to the isolate according to the present invention, and in particular to the use of the Isolate according to the present invention for the preparation of the nutritional formulations

More particularly, the invention relates to the application of these nutritional formulations as beverages, by means of powder mixes to be reconstituted, for dietetic nutrition (sports, slimming), and as ready-to-drink beverages for clinical nutrition (oral route or enteral bag) and dietetic nutrition, in which low viscosity of the beverage and an improvement in the solubility of the pea protein are sought.

The invention also relates to the application of these nutritional formulations as dairy or plant-based beverages, in fermented milks of yoghurt type (stirred, Greek or drinking yoghurt) and as dairy or plant-based creams, iced desserts or sorbets.

Finally, the invention relates to the application of these nutritional formulations as biscuits, muffins, griddle cakes or nutritional bars (intended for specialized/slimming nutrition or sports nutrition), as protein-enriched breads or gluten-free breads, as high protein smell cereals obtained by extrusion cooking (“crisps”), in which high protein solutions are more particularly sought without a negative impact on the preparation process or the texture of the preparations or finished products.

As regards the taste, it was found by the Applicant Company that the undesirable sandy feel in the mouth of the reconstituted powder resulting from the dry mixing of a pea protein in a protein-enriched nutritional formulation in powder form may be reduced or eliminated by implementing a particular pea protein isolate.

It was also found that the incorporation into said nutritional formula of the pea protein isolate of the invention makes it possible to improve the taste of the pea protein by reducing the pea note and the vegetable note.

For the purposes of the invention, the term “nutritional formulations in powder form” means formulations in powder form comprising:
at least one plant protein, and in particular from pea,
optionally, at least one protein of dairy origin, and
optionally, at least one ingredient of fat and carbohydrate type,

which are reconstitutable with an aqueous liquid, and which are suitable for oral administration to a human being.

The term. “dry mixing” as used herein refers, unless otherwise indicated, to the mixing of the components or ingredients to form a base nutritional powder, or to the addition of a dry component in powder or granule form or of a powder-based ingredient to form a nutritional formulation in powder form.

All the percentages, parts and ratios, as used herein, relate to the weight of the total formulation, unless otherwise indicated.

The food formulations in powder form and the corresponding manufacturing processes of the present Invention may comprise, consist of on essentially consist of the essential components of the invention as described herein, and also any additional or optional component described herein or otherwise useful in the applications of the nutritional formulation.

The nutritional formulations in powder form of the present invention comprise a pea protein isolate.

The nutritional formulations in powder form of the present invention are generally in the form of particulate compositions that are capable of flowing or are substantially fluid, or at least particulate compositions that can be readily molded and measured out using a spoon or another similar device, in which the compositions can be readily reconstituted by the intended user with an aqueous solution, typically water, to form a liquid nutritional formulation for immediate oral of enteral use.

In this context, “immediate” use generally means within 48 hours, more typically over about 24 hours, preferably just after reconstitution.

The nutritional formulations in powder form comprise pea protein isolates, which, in certain embodiments, may represent up to 100% of the supplied protein.

The food formulations in powder form may be formulated with all types and amounts of sufficient nutrients so as to form a food supplement, or a specialized nutritional formulation intended to be used by people following a particular diet intended for sports dietetics and slimming.

In one implementation example, the nutritional formulation in powder form may be formulated for a use:
for repairing muscles after an intense effort, for example in the case of sportspeople, or
to ensure the maintenance or construction of muscle mass in sportspeople, or
as a meal substitute for people wishing to lose weight via a satiety-generating effect.

The food formulations in powder form may have a calorific density adapted to the nutritional needs of the final user, although, in the majority of cases, the reconstituted powders comprise from about 350 to about 400 kcal/100 ml.

The food formulations in powder form may have a protein content adapted to the nutritional needs of the final user, although, in the majority of cases, the reconstituted powders comprise from about 20 to about 91 g of protein/100 g, including from about 40 to about 65 g of protein/100 g.

Thus, the formulation may comprise between 20 and 95% of protein relative to the total weight of the formulation, for example between 20-90%, 30-80% or 40-60%.

For example, the pea protein isolate according to the present invention may represent 40-50%, 50-60%, 60-70%, 70-80%, 80-90% or 90-100% of the total protein of the formulation, or any combination of these percentage ranges.

Moreover, the food formulations in powder form may have a fat content adapted to the nutritional needs of the final user, although, in the majority of cases, the reconstituted powders comprise from about 0.5 to about 13 g/100 g, including from about 3 to about 7 g/100 g.

Thus, the formulation may comprise between 0 and 20% of lipids relative to the total weight of the formulation, for example between 0.5-15%, 1-10% or 3-7% (in particular % by weight).

The nutritional formulations in powder form of the present invention may be packaged and sealed in single-use or multi-use containers, and then stored under ambient conditions for up to 36 months or more, more typically from about 12 to about 24 months.

For multi-use containers, they may be opened and covered for repealed use by the final user, on condition that the covered packet is then stored under ambient conditions (for example avoid extreme temperatures) and the contents used within about one or two months.

The fields of application of the nutritional formulations according to the invention are especially:
dietetic nutrition (sports, slimming),
clinical nutrition (in the form of a beverage, a dessert cream or an enteral bag), dairy products (in the form of yoghurts, dairy beverages, dairy creams, iced desserts or sorbets),
high-protein biscuit products, pastry products, breadmaking products and cereal products.

In the sports field, it is known that protein participates in maintaining and growing muscle. The supply of protein is also important for athletes who practice bodybuilding or muscle strengthening.

This protein must be equilibrated in terms of amino acid profile and must comply with the recommendations of the FAO/WHO. Its digestibility is an important factor, going from rapid digestibility to a slower digestibility depending on the moment when the protein is supplied.

Ready-to-drink protein or high-protein beverages then enable the body to benefit from a protein supply of choice, with limited calories.

These high-protein beverages must:
be rich in protein and low in carbohydrates and fat;
have a good taste;
be designed to aid weight loss, by stimulating the loss of fat and by aiding muscle recovery;
be satiety-generating;
help to counter hunger pangs, without added sugars or fat;
have an equilibrated content of essential amino acids, fiber, vitamins and minerals;
be low-calorie.

These ready-to-drink beverages may be advantageously prepared with the pea protein isolates in accordance with the invention. They may moreover be used as sole protein source.

For example, the plant-based beverages that are alternatives to cow's milk contain on average from 4.5 to 11 g of protein per 100 ml of beverage, preferably about 7 g of protein per 100 ml, and are very low in fiber (about 0.5 to 1 g per 100 ml).

Thus, the beverage may comprise between 1 and 20% of protein relative to the total weight of the beverage, for example between 3-15% or 6-8%.

For example, the pea protein isolate according to the present invention may represent 50-60%, 60-70%, 70-80%, 80-90% or 90-100% of the total protein, or any combination of these percentage ranges. Preferably, it represents at least 52%. In particular, the supply of pea protein is between 52 and 100% of the total protein supply.

For ready-to-drink beverages, the supply of pea protein may range from 0 to 100%, preferably from 0.01 or 0.1 to 100%. For example, the pea protein isolate according to the present invention may represent 0.1-10%, 10-20%, 20-30%, 40-50%, 50-60%, 60-70%, 70-80%, 80-90% or 90-100% of the total protein, or any combination of these percentage ranges.

By virtue of a taste free of a pronounced vegetable note, this protein source is well suited to any type of beverage and, by virtue of its moderate viscosity, it may be incorporated at up to 100% without impairing the final taste (although, for very high contents, it may be advantageous to add flavorings).

In the field of “slimming” beverages, i.e. beverages intended to be used in low-calorie diets or intended for weight loss, as mentioned previously, these protein-based or protein-enriched beverages are not only efficient for rapid muscle gain. This type of beverage is also very advantageous in the context of a slimming diet based on protein consumption.

It is known that slimming beverages are ideal for aiding weight loss. They can more particularly:
afford a satiety effect
protect the muscles and tonify the body, avoiding weight regain.

As for “sports” beverages, these slimming beverages have:
an equilibrated content of essential amino acids, fiber, vitamins and minerals
a reduced content of sugars, fat and calories.

Thus, protein-based beverages are indeed of great efficacy for rapidly shedding a few kilos. These protein-rich preparations quite simply reduce or stop the sensation of hunger in the person who consumes them. For example, by taking such a beverage, the user can considerably reduce the amount of food to be consumed, and allow faster weight loss (in the context of a process of replacing meals for weight control, or replacing the total daily ration for weight control).

In clinical nutrition, it is known that enteral nutrition is a therapeutic solution of nutrition by probe which is used when the digestive tube is functional and accessible but when the patient cannot feed normally or else in the case of severe malnutrition.

This technique allows the nutrients to be supplied directly into the digestive tube. If replaces, totally or partly, conventional oral feeding with “complete” nutritive formulations which supply all of the nutrients required by the body.

These formulations are generally packaged in flexible (PVC) bags and administered by means of nasogastric, or gastrostomy, nasojejunal, nasoduodenal or jejunostomy probes.

These nutritional mixes are composed of protein, lipids, carbohydrates, vitamins and minerals, with or without fiber.

Several categories are distinguished: polymer (standard) mixes and semi-elementary (“predigested”) mixes, the latter being indicated in quite specific cases (short bowel syndrome, exocrine pancreatic insufficiency, etc.):

Polymer Mixes
low-calorie (0.5-0.75 kcal/ml), normo or high-protein, with or without fiber
isocalorie (1 kcal/ml), normo or high-protein, with or without fiber
high-calorie (1.25-1.5 kcal/ml), normo or high-protein, with or without fiber
special formulations (glycemic metabolism disorders, respiratory insufficiency).

Semi-elementary mixes ara isocalorie or high-calorie, normo or high-protein mixes, based on medium-chain triglycerides and peptides.

By virtue of their functional properties, pea protein isolates, as a source of protein, are particularly suitable for this use.

Moreover, they make it possible to preserve the same properties as milk protein, for a lower cost.

In the field of the (total or partial) replacement of dairy protein in yoghurts, dairy beverages, dairy creams, ice creams or sorbets, plant protein whose functional properties are equivalent to or even better than those of dairy protein is sought.

In the present patent application, the term “functional properties” means any non-nutritional property which influences the usefulness of an ingredient in a dairy product.

These various properties contribute toward obtaining the desired final characteristics of the dairy product. Some of these functional properties are the solubility, the viscosity, the foaming properties and the emulsifying properties.

Protein also plays an important role in the sensory properties of the food matrices in which it is used, and there is a real synergy between the functional properties and the sensory properties.

The functional properties of protein, or functionalities, are therefore the physical or physicochemical properties which have an effect on the sensory qualities of the food systems generated during technological transformations; storage of domestic culinary preparations.

It is noted that, whatever the origin of the protein, said protein has an influence on the color, the flavor and/or the texture of a product. These organoleptic characteristics have a determining influence on the choice made by the consumer and they are, in this case, strongly taken into account by manufacturers.

The functionality of protein is the result of molecular interactions of the latter with its environment (other molecules, pH, temperature, etc.).

In this instance, it is a matter of the surface properties, which group together the properties of interaction of the protein with other polar or nonpolar structures in the liquid or gas phase: this covers the emulsifying, foaming, etc., properties.

The Applicant Company has noted that there is a real, unsatisfied, need for a nutritional formulation which has advantageous functional properties, and which can be used in dairy product preparation as an at least partial substitute for dairy protein.

By Virtue in particular of their properties of taste improvement, pea protein isolates, as a source of protein, are particularly suitable for this use.

More particularly, in these particular fields of application, i.e.
fermented milks of yoghurt type (stirred, Greek, drinking, etc. yoghurt),
dairy/plant-based beverages,
dairy/plant-based creams (such as “coffee whitener”), iced desserts or sorbets,

the Applicant Company has found that:
As regards “non-dairy coffee whiteners”, also known as “non-dairy coffee creamers”, as will be demonstrated hereinbelow:
   The viscosity of the emulsions after pasteurization before drying is closer to the milk control than pea protein of NUTRALYS® type, which makes it possible to dry a low-viscosity emulsion with a high solids content;
   The flocculation in coffee appears to be less pronounced with the pea protein isolates in accordance with the invention than with pes protein of NUTRALYS® type, but this may be correlated with the improvement of their solubility at the acidic pH of coffee or by better stability to the divalent ions contained in the coffee reconstitution water.
   Moreover, optionally, to improve the flocculating power, it may be chosen to add buffers such as sodium citrates, salts of NaCl type (salt) which promotes protein solubility, or divalent-ion complexing agents that are more efficient than phosphate salts.
   As regards the emulsifying power of said isolates for this particular application, it may be advantageously chosen to use additional emulsifiers, for instance E472 (monoacetyltartaric and diacetyltartaric esters of fatty acid monoglycerides and diglycerides), or to vary the concentrations of E471, or by adjusting the protein concentration or adjusting the homogenization processes.
As regards “stirred yoghurts”, in the manufacturing recipes such as those that will be illustrated hereinbelow,
   The temperature before homogenization may range between 65 to 80° C.,
   The homogenization pressure may range between 150 to 250 bar,
   The pasteurization temperature may range between 80-85° C. for 30 minutes to 90-95° C. for 5 to 10 minutes,
   The fermentation temperature may range between 30 to 45° C., preferentially from 38 to 42° C.,
   It is possible to broaden the type of ferments to all those used in the yoghurt sector as specified in the “yoghurt” regulations.

As regards the formulation of said yoghurts:
In addition to modified starch and pectin, as stabilizers, locust bean or guar gum in various proportions may be chosen,
The starch selected is a modified starch, preferentially starch which develops little viscosity, or even is fully dissolved. Its proportion may range between 2.5 and 5% by weight of the total composition, preferably between 2.8 and 3.5%.
It may be advantageously chosen to add milky flavorings, making it possible to provide a “more dairy” note or fruit preparations, even though the vegetable note is much more attenuated with the pea protein isolates according to the invention with regard to pea protein.
As regards “drinking yoghurts”, the recipes according to the invention are similar to those used for “stirred yoghurts”, but the amount of protein is much lower than that of stirred yoghurts, the amount of starch is preferentially chosen between 1.5% and 2.5% by weight of the total composition, and the starch may or may not be in a dissolved form for this matrix.

In the field of protein enrichment, the supply of calories from protein may prove to be complicated in baking products:
in dry products containing fat such as biscuits, this necessitates the use of a high protein concentration in the formulation. This has a certain impact:
   on the texture of the finished product (increase in hardness/loss of crunchiness, pasty texture)
   on the taste of the finished product (bitterness, bean taste, etc.) and
   on the manufacturing process (fashioning problem/dough rheology/competition for water with the other ingredients, etc.)
in moist products such as bread, the incorporation of protein has an impact on the dough rheology. Since the added protein comes into competition with the gluten network, the consequence of this is a decrease in the bread volume and a more compact and pasty texture.

An even more critical application is the production of high protein crisps, i.e. small cereals obtained by extrusion and intended to be used in inclusion in cereal bars or other cereal agglomerations such as “clusters” or muesli.

A protein content of greater than 70% is sought in these high-protein crisps, which has the consequence of considerably reducing the proportion of starch in the recipe, which is responsible for the expansion and thus the crunchiness. Without these starches, the high-protein crisps are dense and very hard.

Studies have been performed for several years on the functionalities of proteins to select the protein which has the least impact on the texture of protein-enriched baking products.

For the Applicant Company, it is in this context that the pea protein NUTRALYS® BF was developed, since it has low solubility and little interaction with water.

However, this pea protein does not make it possible to fully satisfy the technical problems mentioned above.

Thus, a “protein-rich” biscuit with NUTRALYS® BF is not optimized in terms of texture, the texture remaining pasty.

For high-protein crisps, NUTRALYS® BF does not make it possible, either, to achieve the desired crunchy texture.

In bread, despite an increase in the bread volume after baking, the volume nevertheless remains much lower than that of the control bread.

To solve these difficulties, the Applicant Company thus found that the pea protein isolates in accordance with the invention made it possible:
to improve the solubility relative to pea protein,
to reduce the viscosity in water relative to pea protein.

The pea protein isolates developed have both high solubility and low viscosity, which constitutes a novel combination of properties.

In so doing, the Applicant Company has overcome a technical preconception in that, in order to satisfy the problems of baking products, it was rather necessary to choose a pea protein which has little interaction with water, whereas it turns out that a soluble but sparingly viscous protein performs better.

Nature of the Pea Protein Isolates

The pea protein isolates according to the invention are first characterized by their content of free amino acids (determined according to standard NF EN ISO13903:2005).

This value is between 0.5 and 2%. For example, this value may be between 0.5-1%, 1-1.5% or 1.5-2%, or any combination of these percentage ranges.

For comparative purposes, pea protein (such as NUTRALYS® S85F) has a content of free amino acids of about 0.18%.

The pea protein isolates have a total protein content expressed as N.6.25 of more than at least 70% by weight of dry product, preferably at least 80% by weight, for example between 80 and 99%, 80 and 95%, 80 and 90% or 80 and 85%.

The pea protein isolates according to the invention are also characterized by
their viscosity profile in water at 15% solids and at 20° C. determined as a function of the shear rate;
their solubility profile in water, as a function of the pH, preferably at 20° C.

For the determination of the viscosity profile in water, the measurements are taken
on an aqueous solution of pea protein isolates at 15% solids.
with an AR2000 rheometer from the company TA Instruments,
having concentric cylinder geometry,
with a shear rate of 0.6×10⁻³ at 600 s⁻¹ in 3 minutes (log) and
at a temperature of 20° C. (3 minutes of temperature equilibration before testing).

The shear rates produced in the rheometer make it possible to mimic the treatment conditions to which the solutions of pea protein isolate according to the invention may be subjected:
a shear rate from 1 to 10 s⁻¹ is thus characteristic of a beverage at rest (spoon texture for more viscous products),
a shear rate from 40 to 50 s⁻¹ is the texture in the mouth,
a shear rate of 300-1000 s⁻¹ is equivalent to the shear in product delivery pumps.

Thus, the pea protein isolates in accordance with the invention have a viscosity:
from 11 to 18×10⁻³ Pa·s. at a shear rate of 10 s⁻¹, preferably 12 to 17×10⁻³ Pa·s., ever more preferably from 13 to 16×10⁻¹ Pa·s.,
from 9 to 16×10⁻³ Pa·s. at a shear rate of 40 s⁻¹, preferably 10 to 15×10⁻³ Pa·s., even more preferably from 10 to 14×10⁻³ Pa·s., and
from 8 to 16×10⁻³ Pa·s. at a shear rate of 600 s⁻¹, preferably 9 to 15×10⁻³ Pa·s., even more preferably from 9.8 to 14×10⁻³ Pa·s.

This reflects noteworthy stability of said isolates, irrespective the shear force to which they are subjected.

The pea protein isolates are then characterized by their water solubility profile, as a function of the pH.

The principle of the method used is as follows, as will be developed in the example section:
suspend the pea protein isolate at 2.5% by weight in distilled water,
adjust to the desired pH: in this case 3, 4, 5, 6, 7 or 8 with 0.1 N NaOH or 0.1 N HCl,
mix for 30 minutes at 1100 rpm,
centrifuge for 15 minutes at 3000 g,
measure the solids content of a portion of the supernatant.

The solubility of the pea protein isolates is thus:
from 30 to 40% in pH zones from 4 to 5,
from 40 to 70% in pH zones from 6 to 8,

which reflects their noteworthy solubility within these pH zones.

For comparative purposes, pea protein (such as NUTRALYS® S85F) has:
from 10 to 15% solubility in pH zones from 4 to 5,
from 20 to 50% solubility in pH zones from 6 to 8.

The pea protein isolates are also characterized by their total digestibility profile, with regard to an intact pea protein, and by their digestion kinetics.

As will be illustrated hereinbelow, the digestibility measured in vivo makes it possible to attribute to the pea protein isolates according to the invention a Coefficient of Digestive Use (CDU) with a value of between 93.5 and 95%.

To measure the digestion kinetics of the pea protein isolates, an in vitro model of dynamic digestion under physiological conditions equivalent to the stomach and then the small intestine is used (see example 1, section 4).

As will be illustrated hereinbelow, the behavior of the isolates according to the invention in such a model shows their original positioning between intact pes protein (digestion of “rapid intermediate” type) and whey protein (digestion of “rapid” type).

The pea protein isolates are finally characterized in an in vitro digestibility model as “rapid-digestibility protein”.

To obtain this result, the gastric behavior of five proteins (pea protein, whey protein and sodium caseinates, and two batches of pea protein isolates according to the invention) is evaluated in an in vitro digestion model (see the example on page 32; example 1, section 5).

The digestion kinetics of the proteins depend to a large extent on the residence time in the stomach and on the gastric emptying time.

The viscosity is an important characteristic determining the gastric emptying rate. Thus, in vitro viscosity measurements under gastric conditions are selected as pertinent parameters for characterizing the proteins.

The protein preparations, are introduced into an in vitro system which simulates gastrointestinal digestion, in the present case the system developed by the company NIZO (SIMPHYD system, meaning SIMulation of PHYsiological Digestion) as presented on the website www.nizo.com in their brochure entitled Bioavailability of your ingredients which makes reference to the article published in Appl. Environ. Microbiol. 2007, January; 73(2): 508-15.

This device presents a system of online rheological measurements for comparing the behavior of the test proteins.

The viscosity profiles over time are measured under gastric pH and enzyme release conditions.

As illustrated hereinbelow, when compared with whey protein (classified in the “low viscosity” category) and sodium caseinates (classified among the “prolonged high viscosity” proteins):
pea protein shows a rapid increase in viscosity during acidification, which returns to the baseline at pH 2 (“rapid intermediate viscosity” proteins), whereas
the pea protein isolates according to the invention show a very slight increase in viscosity after acidification, which then decreases to reach values similar to those of whey protein, over 30 minutes (“rapid viscosity” proteins).

Based on their in vitro gastric behavior, the pea protein isolates according to the invention are thus rapidly transported into the duodenum, which will result in rapid assimilation of their amino acids.

Evaluation of the emulsifying properties of the pea protein isolates is performed in comparison with pea protein and milk protein.

It was performed using a Malvern Mastersizer 2000E particle size analyzer via the liquid route.

The measurement principle is based on light scattering.

The powders are dissolved at 1% by weight in azide-containing water with stirring for 6 hours at 750 rpm.

4 ml of edible oil combining four plant oils (sunflower, rapeseed, “oléisol” hybrid sunflower, grapeseed) (for example the off Lesieur Isio 4) are added to 20 ml of proteins (or protein isolate) at 1%.

The whole is blended in a homogenizer (Ultra-Turrax) for 3 minutes at 13 500 rpm, and the emulsions thus formed are then analyzed with a particle size analyzer so as to determine the size of the fat globules thereof.

As will be illustrated hereinbelow, the pea protein isolates according to the invention have better emulsifying properties than the milk protein.

Moreover, their emulsifying property equivalent to that of caseinates makes them most particularly advantageous for the preparation of dried emulsions of “coffee whitener” type.

The present invention relates to the pea protein isolate as described above and to the use thereof for preparing a nutritional formulation.

Preparation of the Pea Protein Isolates According to the Invention

The preparation of the pea protein isolates according to the invention comprises enzymatic or non-enzymatic hydrolysis of the pea protein, so that said pea protein isolate has a degree of hydrolysis (DH) of between 5% and 10%, preferably between 6% and 8% and even more specifically from 6.5% to 7%.

In a first embodiment, the hydrolysis is performed with an endopeptidase.

A nonspecific endopeptidase is chosen, derived from a strain of Aspergillus, in particular a strain of Aspergillus spp of Aspergillus oryzae.

An endopeptidase EC 3-4-11 is more particularly chosen.

The exact amount of enzyme added to the suspension to obtain the desired characteristics of the pea protein isolates will vary as a function of specific characteristics such as:
(1) the enzyme or the enzymatic system used;
(2) the desired final degree of hydrolysis; and/or
(3) the desired molecular weight/final distribution.

Given that these parameters are known, a person skilled in the art can readily determine the appropriate conditions for obtaining the desired characteristics of the pea protein isolate.

In one particular embodiment, the initial pea protein used to prepare the pea protein isolate according to the invention is a pea protein composition as described in patent application WO 2007/17572 or prepared via a process as described in patent application WO 2007/17572 (the teaching being incorporated by reference). In one particular embodiment, the initial pea protein composition is the composition sold by Roquette Frères under the brand name NUTRALYS® S85F.

In a preferred embodiment of the invention, the pea protein suspension is brought to a value of 5 to 20% by weight of solids, in particular from 15 to 20%.

The reaction temperature is adjusted to a value of between 50 and 60° C. preferably about 55° C.

As a general rule, the enzyme system or an enzyme is added to the suspension in amounts in the range from about 0.3 to 1% weight/volume.

The hydrolysis reaction is typically performed over a desired time so as to obtain the desired degree of hydrolysis and/or desired molecular weight profile, in the present case for a time from about 45 minutes to about 2 hours, 30 minutes, preferably about 1 hour.

Once again, the time required for the hydrolysis reaction depends on the characteristics as indicated above, but may be readily determined by a person skilled in the art.

In other embodiments, the suspension containing pea protein may be hydrolysed using non-enzymatic means, for example by mechanical (physical) and/or chemical hydrolysis. This technique is also well known in the prior art.

Once the pea protein has been hydrolyzed to the desired degree the hydrolysis reaction is stopped, for example by inactivating the enzyme, or via other standard means.

In one embodiment, the inactivation of the enzyme is performed by heat treatment.

In accordance with the established practice, the enzyme preparation may be suitably inactivated by increasing the temperature of the incubation suspension to a temperature at which the enzymes become inactivated, for example to about 70° C. for about 10 minutes.

The pea protein isolates thus obtained are then treated at high temperature for a short time (HTST) and then pasteurized and optionally concentrated to a solids content from 10 to 30%, before being dried by atomization. For example, the isolate may be pasteurized at a temperature of between 130° C. and 150° C. for a time from about 1 second to about 30 seconds.

The present invention thus relates to a pea protein isolate that is obtained or that may be obtained via the process as described above.

The present invention also relates to a nutritional formulation comprising a pea protein isolate according to the invention and also to the use of this isolate for preparing a nutritional formulation.

The pea protein isolates according to the invention are present in the nutritional formulation according to the invention in an amount ranging up to 100% by weight, especially in an amount of between 52 and 60% by weight, in particular of the nutritional formulation. For example, the pea protein isolate according to the present invention may represent 0.1-10%, 10-20%, 20-30%, 40-50%, 50-60%, 60-70%, 70-80%, 80-90% or 90-100% of the total protein of the nutritional formulation, or any combination of these percentage ranges.

Moreover, the pea protein isolate according to the present invention may represent 0.1-10%, 10-20%, 20-30%, 40-50%, 50-60%, 60-70%, 70-80%, 80-90% or 90-100% by weight of the nutritional formulation, or any combination of these percentage ranges. Preferably, it represents 0.1-60%, 1-50%, 1-20% or 1-10% or any combination of these percentage ranges.

In one particular embodiment, the pea protein isolate according to the present invention may represent 0.1-10%, 10-20%, 20-30%, 40-50%, 50-60%, 60-70%, 70-80%, 80-90% or 90-100% by weight of the nutritional formulation, or any combination of these percentage ranges, and A may represent 0.1-10%, 10-20%, 20-30%, 40-50%, 50-60%, 60-70%, 70-80%, 80-90% of 90-100% of the total protein of the nutritional formulation, or any combination of these percentage ranges. Preferably, it represents 0.1-60%, 1-50%, 1-20% or 1-10% or any combination of these percentage ranges.

At least part of the pea protein isolates present in the food formulations in powder form is dried by atomization before being introduced (by drying mixing of the like) into the nutritional formulation in powder form.

Nature of the Other Ingredients

The nutritional formulations in powder form may comprise at least one fat, one protein or one carbohydrate, in which at least some of the protein is a pea protein isolate.

The liquid nutritional formulations may comprise at least one protein, carbohydrate and fat, in which at least some of the protein is a pea protein isolate.

In general, a source of fat, carbohydrate and protein, in addition to the pen protein isolate, may be used here, on condition that these macronutrients are also compatible with the essential components of the nutritional formulations according to the invention.

Although the total concentrations or the amounts of fat, protein and carbohydrate may vary according to the user's nutritional needs, these concentrations or amounts usually fall within one of the following ranges, including any other essential fat, protein, carbohydrate and/or ingredients as described herein:
1) for the powder mixes for beverages:
   the fat concentrations are from about 0.5% to about 13%, preferentially from about 1% to about 9%, even more preferentially from about 1% to about 3% by weight of the nutritional formulation in powder form;
   the protein concentrations are from about 20% to about 91%, preferentially from about 40% to about 90%, even more preferentially between about 40% and about 65% by weight of the nutritional formulation in powder form;
   the carbohydrate concentrations are from about 0.9% to about 70%, preferentially from about 2% to about 7%, even more preferentially between about 20% and about 40% by weight of the nutritional formulation in powder form.
2) for the liquids:
   the tat concentrations are from about 1% to about 10%, preferentially from about 1.5% to about 7%, even more preferentially from about 1.5% to about 5% by weight of the nutritional formulation in liquid form;
   the protein concentrations are from about 1% to about 15%, preferentially from about 3% to about 11%, even more preferentially from about 4% to about 7% by weight of the nutritional formulation in liquid form;
   the carbohydrate concentrations are from about 5% to about 45%, preferentially from about 9% to about 20%, even more preferentially from about 13% to about 17% by weight of the nutritional formulation in liquid form.
3) for the dairy products (in the form of yoghurts, dairy beverages, dairy creams, iced desserts or sorbets),
   the fat concentrations are from about 0% to about 15%, preferentially from about 1.5% to about 10%, even more preferentially from about 3% to about 6% by weight of the nutritional formulation in liquid form;
   the protein concentrations are from about 1% to about 25%, preferentially from about 2% to about 20%, even more preferentially from about 2.5% to about 15% by weight of the nutritional formulation in liquid form;
   the carbohydrate concentrations are from about 5% to about 45%, preferentially from about 9% to about 25%, even more preferentially from about 13% to about 20% by weight of the nutritional formulation in liquid form.
4) for the high-protein biscuit products, pastry products, breadmaking products and cereal products:
   the fat concentrations are from about 0% to about 25%, preferentially from about 1% to about 20%, even more preferentially from about 10% to about 18% by weight of the nutritional formulation in liquid form;
   the protein concentrations are from about 1% to about 30%, preferentially from about 2% to about 25%, even more preferentially from about 2.5% to about 15% by weight of the nutritional formulation in liquid form;
   the carbohydrate concentrations are from about 15% to about 75%, preferentially from about 20% to about 60%, even more preferentially from about 20% to about 55% by weight of the nutritional formulation in liquid form.

Nonlimiting examples of fats (in powder or liquid form) or suitable sources thereof for use in the food formulations in powder and liquid form described herein comprise coconut oil, fractionated coconut oil, soybean oil, corn oil, olive oil, safflower oil, safflower oil dich in oleic acid, sunflower oil, sunflower oil rich in oleic acid, palm and palm kernel oils, palm olein, canola oil, marine oils, cotton oils, fats of dairy origin, and combinations thereof.

Nonlimiting examples of carbohydrates or of suitable sources thereof for use in the food formulations in powder and liquid form described herein may comprise maltodextrins, dextrins, corn starch or hydrolyzed or modified corn starch, glucose polymers, corn syrup, carbohydrates derived from rice, glucose, fructose, lactose, high-fructose syrup, honey, sugar alcohols (for example maltitol, erythritol or sorbitol), and combinations thereof.

Nonlimiting examples of proteins, including pea protein isolates, for use in the food formulations in powder and liquid form comprise hydrolyzed, partially hydrolyzed or non-hydrolyzed proteins or protein sources, which may be derived from any known source, such as milk (for example casein or whey), from animals (for example meat or fish), from cereals (for example rice or corn), from oleaginous plants (soybean or rapeseed), seed-bearing leguminous plants (lentils, chickpeas or beans), or combinations thereof.

Nonlimiting examples of such proteins comprise milk protein isolates, milk protein concentrates such as whey protein concentrates, casein, whey protein isolates, caseinates, whole cow's milk, skimmed milk, soybean protein, partially or totally hydrolyzed protein isolates, concentrated soybean protein, and the like.

In one particular embodiment, the nutritional formulation in powder form comprises a combination of a pea protein isolate and of a milk-based protein.

In one example of this particular embodiment, the milk-based protein is present in the nutritional formulation in powder form in an amount of at least 10, 15, 20, 25, 30, 40, 45 or 50% by weight relative to the total weight of protein, preferably about 45% by weight relative to the total weight of protein. For example, the milk-based protein is present in the nutritional formulation in powder form, in an amount of 10-60%, 20-50%, 30-40% by weight relative to the total weight of protein. Preferably, the rest of the protein is provided by the pea protein isolate according to the invention.

In another example of this particular embodiment, the milk-based protein is present in the nutritional formulation in liquid form for clinical nutrition in sin amount of at least 10, 15, 20, 25, 30, 40, 45 or 50% by weight relative to the total weight of protein, preferably about 50% by weight. For example, the milk-based protein is present in the nutritional formulation in liquid form for clinical nutrition in an amount of 10-60%, 20-50%, 30-40% or 45-55% by weight relative to the total weight of protein. Preferably, the rest of the protein is provided by the pea protein isolate according to the invention.

In another example of this particular embodiment, the milk-based protein is present in the nutritional formulation in liquid form for sports in an amount of at least 10, 15, 20, 25, 30, 40, 50, 60 or 75% by weight relative to the total weight of protein, preferably about 75% by weight. For example, the milk-based protein is present in the nutritional formulation in liquid form for sports in an amount of 10-60%, 20-50%, 30-40% or 45-55% by weight relative to the total weight of protein. Preferably, the rest of the protein is provided by the pea protein isolate according to the invention.

Nature of the Optional Ingredients

The nutritional formulations according to the invention may also comprise other ingredients that can modify the chemical, physical, hedonic or processing characteristics of the products or serve as pharmaceutical or additional nutritional components when they are used by certain target populations.

Many of these optional ingredients are known or otherwise adapted for use in other food products and may also be used in the nutritional formulations in accordance with the invention, on condition that these optional ingredients are safe and efficient for oral administration and are compatible with the other essential ingredients of the selected product.

Nonlimiting examples of such optional ingredients comprise preserving agents, antioxidants, emulsifiers, buffers, pharmaceutical active agents, additional nutrients, dyes, flavorings, thickeners and stabilizers, etc.

The nutritional formulations in powder or liquid form may also comprise vitamins or associated nutrients, such as vitamin A, vitamin E, vitamin K, thiamine, riboflavin, pyridoxine, vitamin B12, carotenoids, niacin, folic acid, pantothenic acid, biotin, vitamin C, choline, inositol, salts thereof and derivatives thereof, and combinations thereof.

The nutritional formulations in powder or liquid form may also comprise minerals, such as phosphorus, magnesium, iron, zinc, manganese, copper, sodium, potassium, molybdenum, chromium, selenium, chloride, and combinations thereof.

The nutritional formulations in powder or liquid form may also comprise one or more masking agents to reduce, for example, the bitter tastes in reconstituted powders.

Suitable masking agents comprise natural and artificial sweeteners, sources of sodium, such as sodium chloride, and hydrocolloids such as guar gum, xanthan gum, carrageenan, and combinations thereof.

The amount of masking agent in the nutritional formulation in powder form may vary as a function of the particular masking agent selected, the other ingredients of the formulation and other formulation variables or target products.

Process for Manufacturing the Nutritional Formulation in Powder Form According to the Invention

The base nutrient powder (comprising the pea protein isolate according to the invention) may be prepared by dry-mixing of all the ingredients that are themselves in powder form.

As a variant, the base nutrient powder may be prepared by using conventional wet-route processes which generally comprise the use of two or more suspensions that are finally mixed, processed and then dried.

At least some of the plant protein present in the nutritional formulation in dry-mixed powder form is a pea protein isolate which has advantageously been dried by atomization before being dry-mixed with the nutritional base powder, which generally comprises at least carbohydrates, vitamins and minerals.

In certain embodiments, the pea protein isolate may be processed at high temperature for a short time (HTST) and then pasteurized before being dried by atomization.

More precisely, the pea protein isolate may be added to water and left to hydrate; the water may or may not be heated.

This suspension is then processed by HTST before being dried by atomization. Optionally, the pea protein isolate may be conventionally homogenized after the HTST treatment and before drying by atomization. For example, the isolate may be pasteurized at a temperature of between 130° C. and 150° C. for n time from about 1 second to about 30 seconds.

The step of drying by atomization is a conventional step of drying by atomization which is performed at temperatures and times that are well known and conventional, to produce a plant protein dried by atomization.

Field of Use of the Nutritional Formulations in Powder Form

The dry-mixed nutritional formulations in powder form described and comprising pea protein isolates in accordance with the invention, after reconstitution, have an improved mouthfeel.

An individual can preferably consume at least one portion of the reconstituted nutritional formulation in powder form daily, and, in certain embodiments, can consume two, three or even more portions per day.

Each portion is preferably administered as a single dose, although the portion can also be divided into two or more partial portions, to be taken two or more times in the course of the day.

The nutritional formulations in powder form may be reconstituted for use in infants, children and adults.

The term “about” means the value plus or minus 10%, preferably plus or minus 5%.

### DESCRIPTION OF THE FIGURES

FIG. 1: Particle size distribution of the emulsion which forms the nutritional formulations for clinical nutrition according to the invention

FIG. 2: Sensory analysis of powder mixes formulated with pea protein isolates in accordance with the invention

FIG. 3: Sensory analysis of ready-to-drink beverages for clinical nutrition

FIG. 4: Monitoring of the viscosity during the in vitro digestion of the pea protein isolates according to the invention.

FIG. 5: Solubility profile of the pea protein isolates as a function of the pH

FIG. 6: Sensory analysis of ready-to-drink beverages for sports.

FIG. 7: Sensory analysis of dessert creams for clinical nutrition

FIG. 8: Size distribution of the fat globules of the emulsion prepared with 100% milk protein for an iced dessert preparation

FIG. 9: Size distribution of the fat globules of the emulsion prepared with 50% milk protein and 50% pea protein NUTRALYS® S85F for an iced dessert preparation

FIG. 10: Size distribution of the fat globules of the emulsion prepared with 50% milk protein and 50% pea protein isolate No. 1 according to the invention for an iced dessert preparation

FIG. 11: Size distribution of the fat globules of the emulsion prepared with 50% milk protein and 50% pea protein isolate No. 2 according to the invention for an iced dessert preparation

FIG. 12: melting profile of vegan ice creams prepared with the pea protein isolates according to the invention.

FIG. 13: Sensory analysis of iced desserts

FIG. 14: solubility of the pea protein isolates as a function of the pH in comparison with sodium caseinates

FIG. 15: Sensory analysis of stirred yoghurts—Taste Aspects

FIG. 16: Sensory analysis of stirred yoghurts—Texture Aspects

FIG. 17: Sensory analysis of strawberry-flavored dairy beverages: taste aspect

FIG. 18: Sensory analysis of strawberry-flavored dairy beverages: texture aspect

FIG. 19: Viscosity analysis of chocolate muffin doughs

FIG. 20: Viscosity analysis of griddle cake batters

FIG. 21: Digestibility analysis by monitoring the viscosities using the SIMPHYD device from NIZO

The invention will be understood more clearly with the aid of the following examples which are intended to be illustrative and nonlimiting.

### EXAMPLES

Materials and Methods

Measurement of the DH (Degree of Hydrolysis)

This measurement is based on the method for determining the amino nitrogen on proteins end protein isolates according to the invention with the MEGAZYME kit (reference K-PANOPA) and calculation of the degree of hydrolysis.

Principle:

The “amino nitrogen” groups of the free amino acids of the sample react with N-acetyl-L-cysteine and o-phthalyldialdehyde (OPA) to form isoindole derivatives.

The amount of isoindole derivative formed during this reaction is stoichiometric with the amount of free amino nitrogen. It is the isoindole derivative that is measured by the increase in absorbance at 340 nm.

Procedure:

Introduce an accurately weighed test sample P* of the sample to be analyzed into a 100 ml beaker. (This test sample will be from 0.5 to 5.0 g as a function of the amino nitrogen content of the sample.)

Add about 50 ml of distilled water, homogenize and transfer into a 100 ml measuring cylinder, add 5 ml of 20% SDS and make up to the volume with distilled water; stir for 15 minutes on a magnetic stirrer at 1000 rpm.

Dissolve 1 tablet of flask 1 of the Megazyme kit in 3 ml of distilled water and stir until fully dissolved. Provide one tablet per test.

This solution No. 1 is to be prepared extemporaneously.

The reaction takes place directly in the spectrophotometer cuvettes.
Blank:
Introduce 3.00 ml of solution No. 1 and 50 μl of distilled water.
Standard:
Introduce 3.00 ml of solution No. 1 and 50 μl of flask 3 of the Megazyme kit.
Sample:

Introduce 3.00 ml of solution No. 1 and 50 μl of the sample preparation.

Mix the cuvettes and read the absorbance measurements (A1) for the solutions after about 2 minutes on the spectrophotometer at 340 nm (spectrophotometer equipped with cuvettes with a 1.0 cm optical path, which can measure at a wavelength of 340 nm, and verified according to the procedure described in the manufacturer's technical manual related thereto).

Start the reactions immediately by adding 100 μl of the OPA solution flask 2 of the Megazyme kit to the spectrophotometer cuvettes.

Mix the cuvettes and place them in darkness for about 20 minutes.

Next, read the absorbance measurements for the blank, the standard and the samples on the spectrophotometer at 340 nm.

Calculation Method:

The content of free amino nitrogen, expressed as a mass percentage of product per se, is given by the following formula:

$$[ {NH}_{2} \% crude ] = \frac{( Δ {A}_{sample} - Δ {A}_{blank} ) \times 3.15 \times 14.01 \times V \times 100}{6803 \times 0.05 \times 1000 \times m} = \frac{( Δ {A}_{sample} - Δ {A}_{blank} ) \times 12.97 \times V}{m \times 1000}$$

in which: ΔA=A2−A1
V=volume of the flask
m=mass of the test sample in g
6803=extinction coefficient of the isoindole derivative at 340 nm (in L·mol⁻¹·cm⁻¹).
14.01=molar mass of nitrogen (in g·mol⁻¹)
3.15=final volume in the cuvette (in ml)
0.05=test sample in the cuvette (in ml)

The degree of hydrolysis (DH) is given by the formula:

$$DH = \frac{Protein nitrogen ( \% )}{Amino nitrogen ( \% ) \times 100}$$

in which the protein nitrogen is determined according to the DUMAS method according to standard ISO 16634.

Measurement of the Solubility in Water at Various pH Values

This measurement is based on diluting the sample in distilled water, centrifuging it and analyzing the supernatant.

Procedure:

Introduce 150 g of distilled water at a temperature of 20° C.±2° C. into a 400 ml beaker, mix with a magnetic bar and add precisely 5 g of the lost sample.

Adjust the pH, if necessary, to the desired value with 0.1 N NaOH.

Make up the content with water to 200 g.

Mix for 30 minutes at 1000 rpm and centrifuge for 15 minutes at 3000 g.

Collect 25 g of the supernatant.

Introduce into a predried and tared crystallizing dish.

Place in an oven at 103° C.±2° C. for 1 hour.

Next, place in a desiccator (with dehydrating agent) to cool to room temperature, and weigh.

The content of soluble solids, expressed as a weight percentage, is given by the following formula:

$$\frac{( m 1 - m 2 ) \times ( 200 + P ) \times 100}{P 1 \times P} = \% solubility$$

in which:
   P=weight, in g, of the sample=5 g
   m1=weight, in g, of the crystallizing dish after drying
   m2=weight, in g, of the empty crystallizing dish
   P1=weight, in g, of the sample collected=25 g

Measurement of the In Vitro Digestibility

The SIMPHYD device from NIZO is a static model of simulation of the digestion processes along the gastrointestinal tract.

Gastric digestion is combined with an online viscosity measurement over time. Adapted to physiological conditions, gastric acidification is initiated with concentrated HCl and the enzymes of enzymatic digestion (pepsin and lipase) are added.

All the samples are subjected to the SIMPHYD device at a concentration of 3% (m/V).

The measurements are taken as follows:
A viscosity baseline is determined over 5 minutes, at natural pH and at 37° C.
Acidification to pH 2 is then performed with HCl and the system is maintained at 37° C. for 15 minutes,
Pepsin and lipase are added at 20 minutes.

The viscosity is monitored for 3 hours, using an AR-2000 TA Instruments rheometer at a shear rate of 75 s⁻¹.

The measurements are taken in duplicate. If the difference between two measurements is too large, a third measurement is taken.

The profile of the test proteins is compared with those established by Hall et al. (2003 article entitled Casein and whey exert different effects on plasma amino acid profiles, gastrointestinal hormone secretion and appetite published in Br. J. Nutr. 89: 239-248) for “rapid” and “slow” proteins (whey protein and sodium caseinates, respectively).

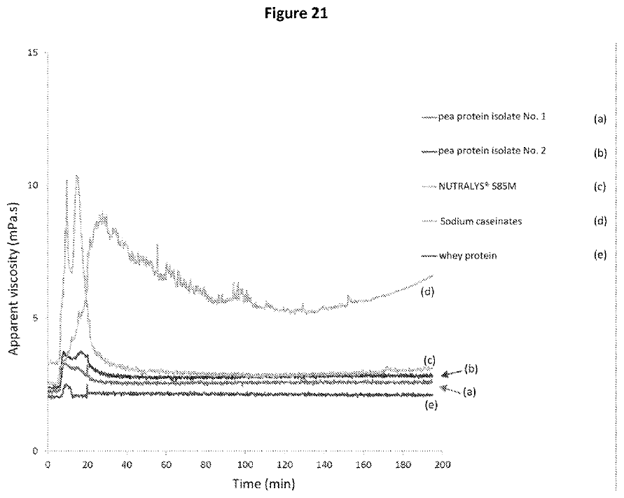

The viscosity profiles obtained are presented in FIG. 21.

The apparent viscosity of the control whey protein sample does not change during the gastric process, whereas the apparent viscosity of the sodium caseinate control increases after gastric acidification and remains high after addition of the digestive enzymes.

After 5 minutes of acidification, the pea protein (NUTRALYS® S85M) shows a first viscosity peak, followed by a second at 15 minutes, and the viscosity profile then rejoins that of the whey protein, at slightly higher values.

The viscosity begins to fall before the addition of the digestive enzymes.

The pea protein isolates according to the invention show a very small increase in apparent viscosity, which decreases again to values slightly above those of the whey protein, for 30 minutes.

The behavior of the pea protein isolates according to the invention reflects their “rapid” nature characteristic of protein that is more satiety-generating than “slow” protein. This induces faster gastric emptying and a post-absorptive increase in plasmatic amino acids.

Measurement of the Emulsifying Power

As indicated above, the measurements are taken by light scattering of redissolved protein powder, the emulsions obtained being analyzed with a particle size analyzer for the size of the fat globules formed.

The results are expressed by:
The Dmode, diameter of the main population,
The D(4.3), arithmetic mean diameter
The D10, D50 and D90, diameters for which there is 10%, 50% and 90% of passage.

The table below collates the size of the fat globules of the emulsions prepared using:
the two pea protein isolates in accordance with the invention, No. 1 and No. 2,
various milk proteins:
a batch of sodium caseinates.

The ΔD corresponds to the difference between the D90 and the D10; it reflects the state of dispersion of the emulsions.

The smaller this value, the closer the droplet sizes, and the more homogeneous the emulsion.

| | Emulsifying capacity: emulsion | | | | | |
|---|---|---|---|---|---|---|
| | size (Dmode in μm) | | | | | Emulsion stability |
| | Dmode | D(4.3) | D10 | D50 | D90 | (ΔD) |
| pea protein isolate No. 1 | 23.4 | 20.5 | 2.1 | 19.4 | 38.4 | 36.3 |
| according to the invention | | | | | | |
| pea protein isolate No. 2 | 23.6 | 21.4 | 3.4 | 20.1 | 39.1 | 35.7 |
| according to the invention | | | | | | |
| Skimmed milk | 24.9 | 20.9 | 5.9 | 20.1 | 36.2 | 30.3 |
| MPC milk protein from | 32.8 | 28.1 | 8.7 | 27.8 | 46.3 | 37.6 |
| FONTERRA | | | | | | |
| MPC 80 Domo milk | 25.5 | 21.8 | 6.5 | 21.1 | 37.3 | 30.8 |
| protein from DMV | | | | | | |
| MPI Prodiet 87B milk | 25.4 | 22.6 | 6.9 | 21.4 | 39.1 | 32.2 |
| protein from INGREDIA | | | | | | |
| Sodium caseinates from | 31.9 | 25.3 | 6.3 | 25.3 | 43.6 | 37.3 |
| DMV | | | | | | |

The pea protein isolates according to the invention have:
good emulsifying properties (lower Dmode: 23.4 and 23.6 μm, respectively)
Emulsion stability (ΔD) of the same order as or even lower than certain concentrated milk proteins or the sodium caseinates and
emulsion homogeneity equivalent to that of milk proteins.

Their properties moreover make them entirely transposable to applications in which a certain level of emulsifying power is required, such as iced dessert preparations or non-dairy coffee whitener, for which caseinates are sought.

### Example 1: Preparation of the Pea Protein Isolates According to the Invention and Characterization of the Pea Protein Isolates Referenced “1” and “2” According to the Invention

Process for Preparing the Pea Protein Isolates No. 1 According to the Invention

1500 kg of pea protein (sold by the Applicant Company under the brand name NUTRALYS® S85F) are mixed into 8500 liters of water preheated to 55° C.

The mixture is stirred for 3 hours at 55° C.

0.5% (weight/weight) of endoprotease FLAVORPRO 750 MDP (from the company BIOCATALYST) is added.

The mixture is stirred for 1 hour at 55° C.

The degree of hydrolysis obtained is then 7.

The reaction is inhibited by heating the medium to 70° C. and keeping it at this temperature for a minimum of 10 minutes.

A UHT treatment is applied (regime: 140° C.—10 seconds).

The mixture is dried by atomization to a solids content of about 93%.

Process for Preparing the Pas Protein Isolates No. 2. According to the Invention

1500 kg of pea protein (sold by the Applicant Company under the brand name NUTRALYS® S85F) are mixed into 8500 liters of water preheated to 55° C.

The mixture is stirred for 3 hours at 55° C.:

0.3% (weight/weight) of endoprotease ENZECO FUNGAL PROTEASE (from the company EDG) is added.

The mixture is stirred for 1 hour at 55° C., and the degree of hydrolysis obtained is than 6.5.

The enzymatic reaction is inhibited by heating the medium to 70° C. and keeping it at this temperature for a minimum of 10 minutes.

A UHT treatment is applied (regime: 140° C.—10 seconds).

The mixture is then dried by atomization to a solids content of about 93%.

Characteristics of the Pea Protein Isolates this Prepared

1. Content of Free Amino Acids

Measurements taken according to standard NF EN ISO13903:2005

| | Free amino acids/ |
|---|---|
| | sum of the amino acids |
| | (g/100 g crude percentage) |
| NUTRALYS ® S85F | 0.18 |
| pea protein isolate No. 1 | 0.77 |
| according to the invention | |
| pea protein isolate No. 2 | 1.85 |
| according to the invention | |

2. Viscosity Profile

For the determination of the viscosity profile in water, the measurements are taken
on an aqueous solution of pea protein isolates containing 15% solids (osmosed and azide-treated water at 200 ppm to prevent any bacteriological risk),
with an AR2000 rheometer from the company TA Instruments,
having concentric cylinder geometry,
with a shear rate of 0.6×10⁻³ at 800 s⁻¹ in 3 minutes (log) and
at a temperature of 20° C. (3 minutes of temperature equilibration before testing).

Before measurement, the solution is stirred for at least 10 hours, at 750 rpm and at 20° C.

The pH is not adjusted.

The following table compares the viscosity profiles of the pea protein isolates in accordance with the invention with those of the control milk proteins and of the pea protein NUTRALYS® S85F.

| Solution containing 15% solids | Viscosity in Pa · s at 20° C., spontaneous pH | | | | | | |
|---|---|---|---|---|---|---|---|
| Sample reference | 5 s⁻¹ | 10 s⁻¹ | 20 s⁻¹ | 40 s⁻¹ | 100 s⁻¹ | 200 s⁻¹ | 600 s⁻¹ |
| Pes protein NUTRALYS ® S85F | 15.820 | 9.538 | 5.700 | 3.570 | 1.000 | 1.251 | 0.680 |
| Milk protein MPC 4882 from | 0.030 | 0.032 | 0.030 | 0.029 | 0.029 | 0.028 | 0.026 |
| FONTERRA | | | | | | | |
| Milk protein Prodiet 27B Fluid | 0.022 | 0.021 | 0.019 | 0.017 | 0.015 | 0.014 | 0.013 |
| from INGREDIA | | | | | | | |
| Pea protein isolate No. 1 | 0.013 | 0.013 | 0.0135 | 0.011 | 0.010 | 0.010 | 0.010 |
| according to the invention | | | | | | | |
| Pea protein isolate No. 2 | 0.015 | 0.016 | 0.016 | 0.014 | 0.013 | 0.014 | 0.014 |
| according to the invention | | | | | | | |

It is found that the pea protein isolates in accordance with the invention show Newtonian behavior, like that of the milk proteins, whereas the pea protein NUTRALYS® S85F shows very pronounced shear-thinning behavior.

Furthermore, the viscosities of the pea protein isolates No. 1 and 2 are very close to the viscosities of the milk proteins, or even lower.

3. Solubility Profile in Water as a Function of the pH

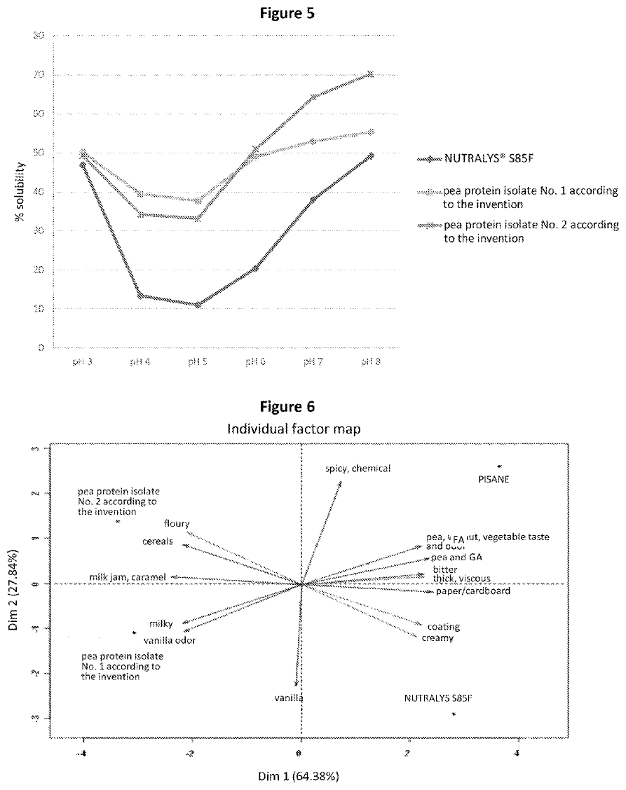

The results are presented in the following table and are illustrated by FIG. 5.

| | | Pea-protein | Pea protein |
|---|---|---|---|
| | Pea protein | isolate No. 1 | isolate No. 2 |
| | NUTRALYS ® | according to | according to |
| Percentage | S85F | the invention | the invention |
| solubility | Mean on 7 samples | | |
| pH 3 | 47 | 50 | 49 |
| pH 4 | 13 | 39 | 34 |
| pH 5 | 11 | 38 | 33 |
| pH 6 | 20 | 49 | 51 |
| pH 7 | 38 | 53 | 64 |
| pH 8 | 49 | 55 | 70 |

4. Stability Study

A study of stability over time of the pea protein isolates in accordance with the invention is conducted so as to measure their behavior with regard to intact pea protein.

The study is conducted after six months of storage according to a temperature/relative humidity regime of:
40° C.±2° C.
at 75%±5% relative humidity.

The measurements are expressed as a percentage loss of solubility (measured according to the above procedure).

| | Pea protein | Pea protein | |
|---|---|---|---|
| | isolate No. 1 | isolate No. 2 | Pea protein |
| | according to | according to | NUTRALYS ® |
| Percentage | the invention | the invention | S85F |
| solubility | After 6 months of storage | | |
| pH 3 | −12.2 | −27.7 | −44.0 |
| pH 4 | −10.8 | −14.8 | −18.4 |
| pH 5 | −6.9 | −9.8 | −5.2 |
| pH 6 | −8.5 | −14.7 | −19.6 |
| pH 7 | −8.8 | −19.9 | −47.4 |
| pH 8 | −5.9 | −22.0 | −57.9 |

It is thus found that, at pH 7, NUTRALYS® S85F loses about half of its solubility, whereas the pea protein isolates lose at most only a fifth of their solubility, and in all cases conserve higher solubility than that of the initial NUTRALYS® S85F.

4—Digestibility Profile

The aim of this study is to evaluate the total protein digestibility of the pea protein isolates No. 1 and 2 according to the invention and to compare it with NUTRALYS® S85F.

For this study, 48 Sprague Dawley rats (Charles River, Lyons, France) weighing 100-125 g at the start of the study were randomized as a function of their weight into four groups of 12 rats.

This experiment was performed in accordance with the European legislation on animal experimentation and with respect for animal well being (APAFIS project No. 0000501).

On their arrival, the rats underwent a 7-day period of quarantine during which they received a standard feed for growing rats.

From the first day of the study, the rats received the following diets, for 10 days:

| | | | Pea protein | Pea protein |
|---|---|---|---|---|
| | | Pea protein | isolate No. 1 | isolate No. 2 |
| | | NUTRALYS ® | according to | according to |
| | Control | S85F | the invention | the invention |
| | in % | in % | in % | in % |
| Test product | 0 | 12.5 | 12.4 | 12.5 |
| Microcrystalline cellulose | 5 | 5 | 5 | 5 |
| from MB Biomedicals | | | | |
| Corn starch | 72.7 | 60.2 | 60.2 | 60.2 |
| qs 100% corn starch | 12.9 | 0.39 | 0.44 | 0.4 |
| Soybean oil-Huileries de | 7.5 | 7.5 | 7.5 | 7.5 |
| Sérignan | | | | |
| Sucrose | 10 | 10 | 10 | 10 |
| Maltodextrin GLUCIDEX ® IT21 | 0 | 0 | 0 | 0 |
| from ROQUETTE FRERES | | | | |
| Choline bitartrate | 0.25 | 0.25 | 0.25 | 0.25 |
| t-Butylhydroquinone | 0.0018 | 0.0018 | 0.0018 | 0.0018 |
| Mineral mixture AIN-93G from | 3.5 | 3.5 | 3.5 | 3.5 |
| MP Biomedicals | | | | |
| Vitamin mixture AIN-93-VX from | 1 | 1 | 1 | 1 |
| MP Biomedicals | | | | |

The amounts being indicated as weight percentages.

The consumption of feed and drink and the weight change are monitored on the first and fifth days of study and then daily up to the tenth and final day of study.

During the first five days of study, the urine and feces are also collected daily. The protein contents of the feeds and feces are determined via the Kjeldahl method (standard ISO 1874:2009).

The nitrogen analyses of the feces and feed make it possible to calculate the Coefficient of Digestive Use (CDU).

$$CDU ( \% ) = \frac{quantity absorbed - quantity excreted in the feces}{quantity absorbed}$$

All the rats had the expected growth. It was significantly lower in the protein-deficient control group, as always in this experimental scheme.

The consumption of drink was not modified by the various diets.

The changes in the other urinary and fecal parameters are directly associated with the control of experimental diet.

As a function of the various experimental days, the following digestibilities were calculated:

| | Digestibility (%) | | | | | | |
|---|---|---|---|---|---|---|---|
| Diet | CDU | D6 | D7 | D8 | D9 | D10 | Mean |
| Pea protein | n | 12 | 12 | 12 | 12 | 12 | 12.00 |
| NUTRALYS ® | mean | 96.5 | 96.3 | 95.3 | 95.0 | 95.8 | 95.8 |
| S85F | standard deviation | 2.6 | 1.4 | 1.4 | 1.7 | 1.9 | 0.8 |
| Pea protein | n | 12 | 12 | 12 | 12 | 12 | 12.00 |
| isolate No. 1 | mean | 94.6 | 94.7 | 93.6 | 92.1 | 93.8 | 93.8 |
| according to | standard deviation | 4.6 | 2.1 | 1.8 | 3.6 | 3.5 | 1.3 |
| the invention | | | | | | | |
| Pea protein | n | 12 | 12 | 12 | 12 | 12 | 12.00 |
| isolate No. 2 | mean | 95.2 | 95.7 | 95.2 | 93.2 | 94.6 | 94.8 |
| according to | standard deviation | 2.9 | 1.9 | 2.1 | 2.8 | 1.8 | 1.0 |
| the invention | | | | | | | |

From a statistical viewpoint, the protein digestibility of NUTRALYS® S85F is significantly different from that of the pea protein isolate No. 1 according to the invention (p=0.0003).

However, from a biological viewpoint, these differences are totally insignificant.

It may thus be concluded that the digestibilities are similar between NUTRALYS and the pea protein isolates No. 1 and No. 2 according to the invention with the following rounding-up:

| Diet | | Global (rounded up) |
|---|---|---|
| Pea protein | mean | 96 |
| NUTRALYS ® | standard | 1 |
| S85F | deviation | |
| Pea protein isolate | mean | 94 |
| No. 1 according to the | standard | 1 |
| invention | deviation | |
| Pea protein isolate | mean | 95 |
| No. 2 according to the | standard | 1 |
| invention | deviation | |

5—Digestion Kinetics

This test uses an in vitro technique of simulation of protein digestion according to the following method.

The use of in vitro digestion methods allows efficient screening of various protein-rich food products as a function of their physicochemical properties and of their behavior during their passage through the stomach and the small intestine.

Here, a comparison is made of 3% (m/m) protein solutions for NUTRALYS® S85F. the pea protein isolates No. 1 and No. 2 according to the invention and the controls commonly used in tests of this type, namely casein and whey.

These five solutions are thus tested in an in vitro model of dynamic digestion under physiological conditions equivalent to the stomach and then the small intestine.

This digestion model is coupled with real-time monitoring of the viscosity using a controlled-stress rheometer (AR-2000, TA Instruments, New Castle, DE, USA) equipped with a stainless-steel fin rotor (height 39 mm and diameter 28 mm).

The protein solutions were tested under the same conditions, namely a regular shear at 0.37° C. and at a rate of 150 s⁻¹ for 3 hours.

The base viscosity was monitored for 5 minutes before performing gradual acidification of the solution down to a pH of between 1.5 and 2.

This acidification generally takes 15 minutes.

Once the pH of the solution has stabilized between 1.5 and 2, an enzymatic cocktail of stomach pepsin (Sigma-Aldrich, St. Louis, MO. USA) and of lipase (Novozyme, Gladesaxe, Denmark) is added. The viscosity monitoring curves are presented in FIG. 4.

Monitoring of the viscosity during the in vitro digestion clearly reflects the digestion kinetics of the proteins. Thus, the digestion of whey does not give rise to a change in the viscosity since it is a rapidly digested protein. Casein, for its part, shows a greatly increased viscosity after acidification, which reflects slow digestion.

The pea protein of NUTRALYS® type demonstrates behavior intermediate between these two standards; it is qualified as being “rapid intermediate”.

However, the pea protein isolates No. 1 and No. 2 according to the invention show behavior that is again intermediate between NUTRALYS® S85F and whey.

It should be noted that the combination of rapid proteins with intermediate proteins may facilitate digestion and prolong the time of diffusion of the amino acids in the blood circulation, which is advantageous for protein synthesis in muscles after a long effort.

### Example 2: Replacement of Milk Protein with the Pas Protein Isolates in UHT-Treated Ready to Drink Beverages for Clinical Nutrition

The nutritive formulations based on milk protein, pea protein and pea protein isolates are presented in the following table:

| | | Nutritional | Nutritional | |
|---|---|---|---|---|
| | | formulation No. 1 | formulation No. 2 | Nutritional |
| | Control | according to the | according to the | formulation with |
| Ingredients | formulation | invention | invention | pea protein |
| Maltodextrin | 17.10 | 17.60 | 17.60 | 17.60 |
| GLUCIDEX ® IT19 | | | | |
| (ROQUETTE FRERES) | | | | |
| Milk protein | 11.00 | 5.53 | 5.53 | 5.53 |
| concentrate (MPC 80- | | | | |
| FONTERRA) | | | | |
| Pea Protein | | | | 5.67 |
| NUTRALYS ® S85F | | | | |
| Pea protein isolate | | 5.67 | | |
| No. 1 (according to | | | | |
| the invention - cf. | | | | |
| example 1 above) | | | | |
| Pea protein isolate | | | 5.67 | |
| No: 2 (according to | | | | |
| the invention - cf. | | | | |
| example 1 above) | | | | |
| Sucrose | 3.20 | 3.4 | 3.4 | 3.4 |
| Rapeseed oil | 4.02 | 3.78 | 3.78 | 3.78 |
| Sunflower oil | 2.68 | 2.52 | 2.52 | 2.52 |
| Soybean lecithin | 0.40 | 0.40 | 0.40 | 0.40 |
| Vanilla flavoring | 0.36 | 0.36 | 0.36 | 0.36 |
| Mineral mixture | 0.492 | 0.273 | 0.273 | 0.273 |
| VITABLEND 7398 | | | | |
| Mineral mixture | | 0.469 | 0.469 | 0.469 |
| VITABLEND 7402 | | | | |
| Demineralized water | 60.75 | 60 | 60 | 60 |
| Total | 100 | 100 | 100 | 100 |

The amounts being indicated as weight percentages.

Their nutritional values per 100 ml are as follows:

| | | Nutritional | Nutritional | |
|---|---|---|---|---|
| | | formulation No. 1 | formulation No. 2 | Nutritional |
| | | according to the | according to the | formulation with |
| | Control formulation | invention | invention | pea protein |
| Calorific energy (kCal) | 204 | 203 | 203 | 198 |
| Protein content (g) | 10.0 | 10.0 | 10.0 | 9.75 |
| Of which milk protein | 10.0 | 5.0 | 5.0 | 4.9 |
| Pea protein isolates | 0 | 5.0 | 5.0 | 0 |
| Pea protein | 0 | 0 | 0 | 4.9 |
| Fat (g) | 8.0 | 8.0 | 8.0 | 7.85 |
| Carbohydrates (g) | | | | |
| Of which sucrose | 5.1 | 5.1 | 5.1 | 5.0 |
| Fiber (g) | | | | |
| Of which soluble | 0.0 | 0.0 | 0.0 | 0.0 |
| Of which insoluble | 0.0 | 0.1 | 0.1 | 0.1 |

Mineral composition (per 100 ml):

| | | Nutritional |
|---|---|---|
| | | formulation No. 2. |
| | | according to the |
| (mg) | Control formulation | invention |
| Na | 67.8 | 107.7 |
| Ca | 242.0 | 241.4 |
| K | 163.9 | 160.4 |
| Cl | 65.0 | 64.1 |
| P | 154.0 | 135.3 |
| Mg | 25.4 | 24.9 |

The process for manufacturing the beverages is as follows:
Dry mix all the powders (milk, protein, pea protein, pea protein isolates, maltodextrines and sucrose),
Weigh out 90% of the water at 55° C.,
Add the powder mix to the water at 55% ° C., disperse with a whip for 1 minute and then mix with SILVERSON blender for 30 minutes at 55° C.,
In a separate mixing container, dissolve the minerals with the remaining water at 50° C. and add immediately to the solution,
Add the vanilla flavoring to said solution,
Place the lecithin and the oil in a separate mixing container, stir and heat to about 55° C.,
After 35 minutes of hydration, add the lecithin and the oil mixture to the main patch, using a shear of 10 000 rpm for 5 minutes,
Preheat the product to 75° C. per batch of 3 liters in a beaker on a water bath (temperature rise of about 10 minutes) and homogenize at 200 bar (one stage) on a homogenizer of 10 liters/hour (about 20 minutes per batch),
Cool the product to 30° C. for 5 minutes to 45 minutes depending on the batch,
Adjust to pH 6.8-7, at 30° C., with 30% sodium hydroxide,
Heat the product again on a water bath at 75° C. (temperature rise over 10 minutes),
Sterilize the product at 142° C. for 5 seconds in a tubular exchanger and then cool in the exchanger to 25° C.,
Package the product in bottles,
Store at +4° C.

The analyses performed on the formulation are as follows:

1. Particle Size Analysis on the Emulsion Formed by the Nutritional Formulations According to the Invention

The object here is to analyze the aspect of the pea protein isolate-based nutritional formulations with regard to the milk protein-based control.

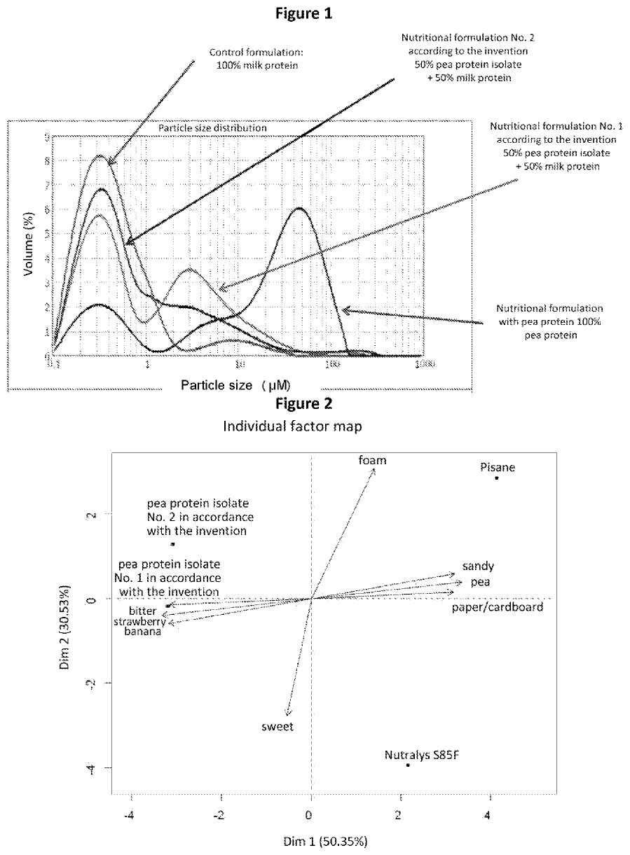

The analyses were performed with a reference 2000 particle size analyzer from MALVERN. The results obtained are presented in FIG. 1 and in the following table (the values in the table are the mean of three measurements).

The Dmode is the main particle diameter. The d10, d50 and d90 are the particle diameter values representing, respectively, 10%, 50% and 90% of the total particles.

| | Particle size (μm) | | | |
|---|---|---|---|---|
| | After homogenization and before the heat | | | |
| | treatment step | | | |
| | Dmode | d10 | d50 | d90 |
| Control formulation: 100% | 0.3 | 0.2 | 0.4 | 1.4 |
| milk protein | | | | |
| Nutritional formulation No. | 0.3 | 0.2 | 0.9 | 10.3 |
| 1 according to the | | | | |
| invention | | | | |
| 50% pea protein isolate | | | | |
| No. 1 + 50% milk protein | | | | |
| Nutritional formulation No. | 0.3 | 0.2 | 0.5 | 6.7 |
| 2 according to the | | | | |
| invention | | | | |
| 50% pea protein isolate | | | | |
| No. 2 + 50% milk protein | | | | |
| Nutritional formulation with | 46.1 | 0.3 | 25.4 | 78.4 |
| pea protein | | | | |
| 100% pea protein | | | | |

The four samples show a bimodal particle distribution. The first peak (first family of particles), centered on 0.3 μm, is predominant in the first three formulations. For the nutritional formulation with pea protein, this population is minor.

The second peak of the bimodal distribution (second family of particles) depends on the sample:
for formulations 1 and 2 according to the invention, there is a population centered on 3 μm, but the volume of said population is greater for formulation No. 1;
for the control formulation: the second peak is centered on 10 μm;
the nutritional formulation with pea protein shows its difference by having a second peak centered on 48 μm.

It is deduced therefrom that the pea protein isolates No. 1 and No. 2 make it possible to obtain emulsion sizes for the beverage close to that obtained with milk protein.

Moreover, the pea protein isolate No. 2 even gives a better emulsion size distribution (less bimodal distribution and better emulsion stability represented by the difference between the D90 and D10) than the pea protein isolate No. 1.

2. Viscosity Analysis

The object here is to show the stability of the pea protein isolate-based nutritional formulations with regard to the milk protein-based control, and also to demonstrate the technological advantage in choosing these isolates with regard to pea protein.

The measuring parameters are as follows:
Rheometer: Physica MCR301
Tools: concentric cylinder CC27
Temperature: 20° C. (5 minutes to be at equilibrium)
Shear: 0.05 to 1000 s⁻¹ in 6 minutes

The result of the viscosity measurements is presented in the following table (the pea protein isolate No. 1 according to the invention is analyzed here):

| | Viscosity in Pa · s at 20° C. | | | | | | | | |
|---|---|---|---|---|---|---|---|---|---|
| | | | | | | | Affor 1 month of | | |
| | Before heat treatment | | | After heat treatment | | | storage at +4° C. | | |
| | 10 s⁻¹ | 100 s⁻¹ | 1000 s⁻¹ | 10 s⁻¹ | 100 s⁻¹ | 1000 s⁻¹ | 10 s⁻¹ | 100 s⁻¹ | 1000 s⁻¹ |
| Control formulation: | 0.125 | 0.107 | 0.088 | 0.046 | 0.045 | 0.044 | 0.055 | 0.051 | 0.047 |
| 100% milk protein | | | | | | | | | |
| Nutritional | 0.063 | 0.053 | 0.046 | 0.079 | 0.053 | 0.040 | 0.327 | 0.151 | 0.099 |
| formulation No. 1 | | | | | | | | | |
| according to the | | | | | | | | | |
| invention | | | | | | | | | |
| 50% pea protein | | | | | | | | | |
| isolate No. 1 + 50% | | | | | | | | | |
| milk protein | | | | | | | | | |
| Nutritional | 0.105 | 0.097 | 0.087 | 1.160 | 0.421 | 0.183 | 1.110 | 0.392 | 0.169 |
| formulation with pea | | | | | | | | | |
| protein | | | | | | | | | |
| 100% pea protoin | | | | | | | | | |

The heat treatment does not affect the three nutritional formulations in the same way:
For the control formulation, the heat treatment leads to a decrease in viscosity,
For formulation No. 1, there is an increase in viscosity and increase in the shear-thinning behavior, but this viscosity increase remains low and is close to that of the control formulation.

It is in fact found that, before the heat treatment, the control formulation has the highest viscosity, followed by the pea protein-based formulation.

After the heat treatment, on the other hand, the control formulation has the lowest viscosity, followed by formulation No. 1 in accordance with the invention.

In conclusion, the nutritional formulation No. 1 according to the invention and the milk protein-based control formulation have rheological behavior that is similar in terms of viscosity and heat resistance.

Other viscosity measurements were taken on the same beverage recipes, this time also taking a nutritional formulation prepared with the pea protein isolate No. 2 (especially changing the cooking method, in this case UHT sterilization (142° C.—5 seconds) at 20 liters/hour online with in-phase homogenization descending to 75° C. at 200 bar after the heat treatment) and after one month of storage of the beverages at +4° C.

| | Viscosity in Pa · s at 20° C. | | | | | |
|---|---|---|---|---|---|---|
| | | | | After 1 month of | | |
| | After heat treatment | | | storage at +4° C. | | |
| | 10 s⁻¹ | 100 s⁻¹ | 1000 s⁻¹ | 10 s⁻¹ | 100 s⁻¹ | 1000 s⁻¹ |
| Control formulation: | 0.049 | 0.047 | 0.045 | 0.085 | 0.076 | 0.088 |
| 100% milk protein | | | | | | |
| Nutritional formulation | 0.101 | 0.073 | 0.057 | 0.157 | 0.097 | 0.085 |
| No. 1 according to the | | | | | | |
| invention | | | | | | |
| 50% pea protein isolate | | | | | | |
| No. 1 + 50% milk | | | | | | |
| protein | | | | | | |
| Nutritional formulation | 0.107 | 0.075 | 0.057 | 0.155 | 0.095 | 0.064 |
| No. 2 according to the | | | | | | |
| invention | | | | | | |
| 50% pea protein isolate | | | | | | |
| No. 2 + 50% milk | | | | | | |
| protein | | | | | | |
| Nutritional formulation | 0.963 | 0.343 | 0.159 | 1.47 | 0.459 | 0.202 |
| with pea protein | | | | | | |
| 100% pea protein | | | | | | |

These results show that all the formulations have a viscosity which increases after one month at 4° C. Formulations No. 1 and 2 have a viscosity which increases very slightly, of the same order of magnitude as that of the control formulation, after one month, in comparison with the pea protein formulation.

The beverages containing the pea protein isolates No. 1 and 2 are much more stable than the beverage containing the pea protein, and approach the stability of the beverage containing milk protein.

### Example 3: Replacement of Milk Protein with the Pea Protein Isolates in UHT-Treated Ready-to-Drink Beverages for Sports Nutrition

The nutritional formulations have the following compositions:

| | Recipe No: | | | | |
|---|---|---|---|---|---|
| | 1 | 2 | 3 | 4 | 5 |
| | % by | % by | % by | % by | % by |
| | weight | weight | weight | weight | weight |
| Milk protein concentrate | 8.05 | 4.85 | 4.85 | 4.85 | 4.85 |
| (MPC 80 DMV) | | | | | |
| Pea protein isolate No. 1 (according to | | 3.30 | | | |
| the invention - cf. example 1 above) | | | | | |
| Pea protein isolate No. 1 (according to | | | 3.30 | | |
| the invention - cf. example 2 above) | | | | | |
| Pea protein NUTRALYS ® S85F | | | | 3.30 | |
| (ROQUETTE FRERES) | | | | | |
| Pea protein PISANE ® from the | | | | | 3.30 |
| company COSUCRA | | | | | |
| Maltodextrin GLUCIDEX ® IT19 | 1.70 | 1.60 | 1.60 | 1.60 | 1.60 |
| (ROQUETTE FRERES) | | | | | |
| Sucrose | 3.80 | 4 | 4 | 4 | 4 |
| Vanilla flavoring | 0.36 | 0.36 | 0.36 | 0.36 | 0.36 |
| MATVIS C3000 (AGI) | 0.04 | 0.04 | 0.04 | 0.04 | 0.04 |
| Liquid skimmed milk | 86.05 | 85.85 | 85.85 | 85.85 | 85.85 |
| TOTAL | 100 | 100 | 100 | 100 | 100 |

The amounts being indicated as weight percentages.

Their nutritional values per 100 ml are as follows:

| Recipe No. | 1 | 2 | 3 | 4 | 5 |
|---|---|---|---|---|---|
| Calorific energy (kCal) | 86 | 86 | 86 | 86 | 86 |
| Protein content (g) | 10 | 10 | 10 | 10 | 10 |
| Of which milk protein | 10 | 7.2 | 7.2 | 7.2 | 7.2 |
| Pea protein isolates | 0 | 2.8 | 2.8 | 0 | 0 |
| Pea protein | 0 | 0 | 0 | 2.8 | 2.8 |
| Fat (g) | 0.2 | 0.5 | 0.5 | 0.5 | 0.5 |
| Carbohydrates (g) | 10.5 | 10.5 | 10.5 | 10.5 | 10.5 |
| Of which sugars | 8.5 | 8.5 | 8.5 | 8.5 | 8.5 |
| Fiber (g) | 0 | 0.1 | 0.1 | 0.1 | 0.1 |
| Of which soluble | 0 | 0 | 0 | 0 | 0 |
| Of which insoluble | 0 | 0.1 | 0.1 | 0.1 | 0.1 |

The conditions for preparing said beverages are the same as those of Example 2.

Viscosity measurements are taken on said ready-to-drink sports beverages.

The object here is also to show the stability of the pea protein isolate-based nutritional formulations with regard to the milk protein-based control, and also to demonstrate the technological advantage in choosing these isolates with regard to pes protein.

The measuring parameters are as follows:
Rheometer: Physica MCR301
Tools: concentric cylinder CC27
Temperature: 20° C. (5 minutes to be at equilibrium)
Shear: 0.05 to 1000 s⁻¹ in 6 minutes

The result of the viscosity measurements is presented in the following table:

| | | Viscosity (Pa s) at | | |
|---|---|---|---|---|
| Recipe reference | heat treatment | 10 s⁻¹ | 100 s⁻¹ | 100 s⁻¹ |
| 1 | Before UHT | 0.74 | 0.27 | 0.09 |
| | After UHT | 0.27 | 0.09 | 0.043 |
| 2 | Before UHT | 0.067 | 0.043 | 0.028 |
| | After UHT | 0.024 | 0.02 | 0.018 |
| 3 | Before UHT | 0.106 | 0.056 | 0.032 |
| | After UHT | 0.026 | 0.021 | 0.019 |
| 4 | Before UHT | 0.04 | 0.027 | 0.02 |
| | After UHT | 0.15 | 0.075 | 0.042 |
| 5 | Before UHT | 0.042 | 0.029 | 0.022 |
| | After UHT | 0.091 | 0.055 | 0.035 |

It is deduced therefrom that:
before UHT, the formulations containing pea protein isolates have a viscosity that is intermediate between the milk protein and the pea protein,
after UHT, the formulations containing pes protein isolates lose viscosity, whereas the pea protein gains viscosity, the milk protein remaining the most viscous.

The pea protein isolates are thus more stable to heat treatment than the pea protein and are more suited to UHT ready-to-drink sports beverages on account of their low viscosity, which are properties required for UHT ready-to-drink sports beverages.

### Example 4: Replacement of Milk Protein with the Pea Protein Isolates in a UHT-Treated Liquid Nutritional Formulation for Enteral Clinical Nutrition

The nutritional formulations then have the following compositions:

| | 1 | |
|---|---|---|
| | Control | 2 |
| | 100% calcium | 50% Substitution |
| | caseinate | of milk protein |
| Calcium caseinate (DMV) | 4.96 | 2.48 |
| Pea protein isolate No. 1 or No. 2 | 0.00 | 2.82 |
| Maltodextrin GLUCIDEX ® IT 19 | 14.60 | 14.60 |
| Rapeseed oil | 4.40 | 4.40 |
| Water | 75.04 | 74.70 |
| Mineral mix Vitablend | 1.00 | 1.00 |
| TOTAL | 100.00 | 100.00 |

The amounts being indicated as weight percentages.

The nutritional values per 100 ml are as follows,

| Recipe No. | 1 | 2 |
|---|---|---|
| [symbol] (%) | 24.2 | 24.5 |
| [symbol] (kCal) | 116 | 117 |
| per 100 [symbol] | | |
| Fat | 4.5 | 47 |
| [symbol] | | |
| Protein content | | |
| [symbol] | 4.52 | 4.52 |
| Of which milk prote | 4.52 | 2.26 |
| Pea protein isolates | 0 | 2.26 |
| Carbohydrates | | |
| [symbol] | 14 | 14 |
| Of which sugars | 0.9 | 0.9 |
| Fibers | | |
| [symbol] | 0 | 0 |
| Of which soluble | 0 | 0 |
| Of which insoluble | 0 | 0 |

[symbol]

indicates data missing or illegible when filed

The process for manufacturing the beverages is as follows:
Mix the caseinates in water at 50° C., add the proteins, and mix on a magnetic plate for 10 minutes,
Add the carbohydrates and the minerals while mixing with a SILVERSON blender,
Add the oil while mixing with a SILVERSON blender for 5 minutes (10 000 rpm),
Homogenize at 60° C. at 250 bar in a NIRO SOAVI 2K high-pressure homogenizer (2 stages),
Adjust the pH to 6.9 with 50% citric acid solution,
Sterilize in an autoclave in glass jars at 120° C. for 15 minutes.
Cool to room temperature.

Under these operating conditions, the pea protein isolates may be advantageously used in replacement for milk protein.

### Example 5: Comparison of the Sensory Properties of Four Powder Mixes Formulated with Pea Protein Isolates in Accordance with the Invention for Sportspeople

The panel consisted of 13 people.

The panel is qualified for tasting products formulated with pea protein. It received training so as to check its performance in terms of:
Capacity to discriminate the products
Consensus, correct use of the descriptors
Repeatability, ability to detect a product submitted twice

Specifically, it received training in the correct use of the sensory descriptors of taste and texture, for instance:

| Descriptor | Definition | Procedure | Reference |
|---|---|---|---|
| PEA | Typical pea taste | Taste the product | solution containing 3% by |
| | | | weight of garden peas in |
| | | | water ( [symbol] with a blender) |
| STRAWBERRY | Typical strawberry | Taste the product | strawberry-flavored |
| | taste | | solution |
| BANANA | Typical banana taste | Taste the product | banana-flavored |
| | | | solution |
| CARDBOARD | Typical cardboard | Taste the product | Solution of [symbol] acid at 0.5 |
| | taste | | g/liter in mineral water |
| SWEET | Sweet taste | Taste the product | Solution of sucrose at 6 |
| | | | g/liter in mineral water |
| BITTER | Bitter taste | Taste the product | Solution of caffeine at 0.5 |
| | | | g/liter in mineral water |
| SANDY | Evaluation of the | Chew a product unit until it is | Certain [symbol] |
| | granular nature and of | ready to swallow and evaluate | |
| | the number of | the perception of small grains | |
| | particles of a product | in the mouth | |
| | (not sandy/ | | |
| | very sandy) | | |

[symbol]

indicates data missing or illegible when filed

The method also allows them to make comments on other descriptors that were not anticipated in this list.

Products

The nutritional formulations are powder mixes intended for sportspeople, having the following composition:

| Ratio of pea protein or pea protein | 55/45 | 55/45 | 55/45 | 55/45 |
|---|---|---|---|---|
| isolates according to the | | | | |
| invention/milk protein | | | | |
| Nutritional formulations | Pea protein | Pea protein | Pea protein | Pea protein |
| based on: | NUTRALYS [symbol] | Isolate No. 1 | Isolate No. 2 | PISANE [symbol] |
| | S85F | according | according | from the |
| | | to the | to the | company |
| | | invention | invention | COSUCRA |
| | % by weight | % by weight | % by weight | % by weight |
| Whey protein concentrate | 43.2 | 43.2 | 43.2 | 43.2 |
| (WPC 80 [symbol] ) | | | | |
| Pea protein isolate No. 1 according | 0 | 52.8 | | |
| to the invention | | | | |
| Pea protein isolate No. 2 according | 0 | 0 | 52.8 | 0 |
| to the invention | | | | |
| Pea protein PISANE [symbol] from the | 0 | 0 | 0 | 52.8 |
| company COSUCRA | | | | |
| NUTRALYS [symbol] S85F | 52.8 | 0 | 0 | 0 |
| Banana flavoring (N-Capture MSD | 0.18 | 0.18 | 0.18 | 0.18 |
| M [symbol] 0058032) | | | | |
| Strawberry flavoring (N-Capture | 2.7 | 2.7 | 2.7 | 2.7 |
| MSD M [symbol] 0055259) | | | | |
| Salt | 0.5 | 0.5 | 0.5 | 0.5 |
| Xanthan gum F80 (AGI) | 0.35 | 0.35 | 0.35 | 0.35 |
| [symbol] K | 0.1 | 0.1 | 0.1 | 0.1 |
| Sucralose | 0.1 | 0.1 | 0.1 | 0.1 |
| TOTAL | 100 | 100 | 100 | 100 |

The amounts being indicated as weight percentages. [symbol]
indicates data missing or illegible when filed

They are reconstituted in water at room temperature just before tasting.

Tasting Conditions:
In a sensory analysis laboratory: individual tasting cubicles, white walls, calm environment (to facilitate concentration)
White light (to have exactly the same vision of the product)
At the end of the morning or the afternoon (to be at the height of the sensory capacities)
Products rendered anonymous with a three-figure code (to prevent the code from influencing the assessment of the products)
Products presented in a random order (to prevent order and persistence effects)

Test

The method employed to compare the results was the Flash Profile (J. M. Sieffermann, 2000—Le profil Flash: Un outil rapide et innovant d'évaluation sensorielle descriptive. [The Flesh Profile: a rapid and innovative tool for descriptive sensory evaluation] In: L'innovation: de l′idée au success [Innovation: from the idea to success]—12th AGORAL Meeting. Pages 335-340, March 22-23, 2000. Paris, France: Lavoisier, Tec & Doc.).

The products are all presented simultaneously. It is a matter of comparing the products by making a succession of classifications: the panelists choose the descriptors which appear to them to be the most pertinent to discriminate between the products, and classify the products according to these descriptors; it is possible that several products are grouped in the same row.

### Example

Sensory descriptor: Crunchy

Data Processing

The statistical processing method suited to this type of data is multiple factor analysis (J. Pagès, 1894—Multiple factor analysis (AFMULT package). In: Computational Statistics & Data Analysts, Volume 18, Issue 1; August 1994, Pages 121-140) on the data-rows of the products.

In order for the results to be clearer, the MFA was performed several times; globally, and per criterion (aspect, odor, taste, texture). The graphs presented summarize all of the results provided by this method.

The analyses were performed using the R software (on open sale):
R version 2.14.1 (2011-12-22)
Copyright (C) 2011 The R Foundation for Statistical Computing
ISBN 3-900051-07-0
20 Platform: i386-pc-mingw32/1386 (32-bit)

The software is a working environment which requires the loading of modules containing the calculation functions such as the FactoMineR version 1.19 package.

Results

Representation of the results in graph form is presented in FIG. 2.

Three groups are distinguished: the pea protein PISANE® sold by the company COSUCRA, the pea protein NUTRALYS® S85F, and the two pea protein isolates in accordance with the invention of example 1.

The panelists established little difference between the two pea protein isolates in accordance with the invention of example 1.

They have a less sandy, pea and paper/cardboard aspect than the control products and are more bitter and more strawberry/banana (flavors used in this formulation).

The mix with PISANE® stands out in terms of texture since its application generated the formation of foam.

The mix with NUTRALYS® S85F, for its part, stood out for its sweet taste.

### Example 6: Comparison of the Sensory Properties of Ready-to-Drink Beverages for Clinical Nutrition

The panel consisted of 14 people.

The panel, as in example 3, is qualified for tasting products formulated with pea protein. It received training so as to check its performance in terms of:
Capacity to discriminate the products
Consensus, correct use of the descriptors
Repeatability, ability to detect a product submitted twice

Specifically, it received training in the correct use of the sensory descriptors of taste and texture, for instance:

| Descriptor | Definition | Procedure | Reference |
|---|---|---|---|
| PEA | Typical pea taste | Taste the product | solution containing 3% by |
| | | | weight of garden peas in |
| | | | water ( [symbol] with a mixer) |
| VANILLA | Typical vanilla taste | Taste the product | Vanilla-flavored solution |
| CARAMEL | Typical caramel taste | Taste the product | Caramel |
| SALTY | Salty taste | Taste the product | Solution of sodium chloride |
| | | | at 0.01 g/liter in mineral |
| | | | water |
| ASTRINGENT | Sensation of dryness | Taste the product | Solution of tannic acid [symbol] |
| | | | 0.5 g/liter in mineral water |
| CREAMY/ | Evaluation of the soft | Chew the product unit and | [symbol] , thick cream |
| TOPPING | and fondant texture of | check whether it causes a soft | |
| | the product | contact and whether [symbol] | |
| | (not creamy/ | the mouth [symbol] | |
| | very creamy) | | |
| TACK | Evaluation of the | After chewing consecutively a | Soft caramel |
| | force required to | few times, press the product | |
| | detach products that | between the teeth and | |
| | adhere inside the oral | measure the force required for | |
| | cavity (not tacky/ | it to be detached from the | |
| | very tacky) | teeth | |
| THICK | Evaluation of the | Rub the tongue against the | Sweetened concentrated |
| | ease of the product | palate and check whether the | milk, honey |
| | flow in the mouth, | product flows easily [symbol] | |
| | (not thick/very thick) | | |

[symbol]

indicates data missing or illegible when filed

The method also allows them to make comments on other descriptors that were not anticipated in this list.

Products

The products are ready-to-drink beverages, the recipes for which are those of example 2.

They are presented to the panelists at room temperature.

Tasting Conditions:
In a sensory analysis laboratory: individual tasting cubicles, white walls, calm environment (to facilitate concentration)
White light (to have exactly the same vision of the product)
At the end of the morning of the afternoon (to be at the height of the sensory capacities)
Products rendered anonymous with a three-figure code (to prevent the code from influencing the assessment of the products)
Products presented in a random order (to prevent order and persistence effects)

Exercise

The method employed to compare the products was the Flash Profile (J. M. Sieffermann, 2000).

The products are all presented simultaneously. It is a matter of comparing the products by making a succession of classifications: the panelists choose the descriptors which appear to them to be the most pertinent to discriminate between the products, and classify the products according to these descriptors; it is possible that several products are grouped in the same row.

### Example

Sensory descriptor: Crunchy

Data Processing

The statistical processing method suited to this type of data is multiple factor analysis (J. Pagès, 1994) on the data-rows of the products. In order for the results to be clearer, the MFA was performed several times; globally, and per criterion (aspect, odor taste, texture). The graphs presented summarize all of the results provided by this method.

The analyses were performed using the R software (on open sale):
R version 2.14.1. (2011-12-22)
Copyright (C) 2011 The R Foundation for Statistical Computing
ISBN 3-900051-07-0
20 Platform: i386-pc-mingw32/1366 (32-bit)

The software is a working environment which requires the loading of modules containing the calculation functions such as the FactoMineR version 1.19 package.

Results

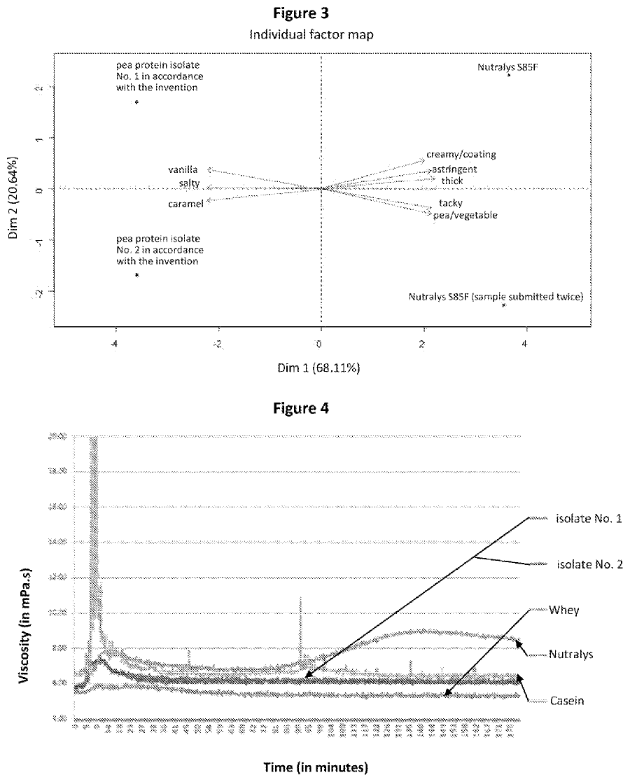

Representation of the results in graph form is presented in FIG. 3.

NUTRALYS® S85F was presented twice to test the repeatability of the panel. It may be seen on the graph that the two points are close on the first dimension (the largest) but not on the second; it is thus considered that this second dimension consists of measurement noise. Thus, there is no significant difference between the two pea protein isolates in accordance with the invention, since they are close on the first dimension.

It is seen that the two pea protein isolates in accordance with the invention are more vanilla/caramel and sally than NUTRALYS® S85F, which proves to be more pea/Vegetable and astringent in taste; more creamy/coating, thick and tacky in texture.

### Example 7: Comparison of the Sensory Properties of Ready-to-Drink Beverages for Sportspeople

The panel consisted of 12 people.

The panel, as in example 3, is qualified for fasting products formulated with pea protein. It received training so as to check its performance in terms of:
Capacity to discriminate the products
Consensus, correct use of the descriptors
Repeatability, ability to detect a product submitted twice

Products

The products are ready to drink beverages, the recipes for which are those of example 3. They are presented to the panelists at room temperature.

Exercise

The method employed to compare the products was the Flash Profile (J. M. Sieffermann, 2000).

The products are all presented simultaneously. It is a matter of comparing the products by making a succession of classifications: the panelists choose the descriptors which appear to them to be the most pertinent to discriminate between the products, and classify the products according to these descriptors; it is possible that several products are grouped in the same row.

### Example

Sensory descriptor: fresh walnut

Here is the list of descriptors presented to the panelists as a guide:

| flavor/odor | | | | taste/ | |
|---|---|---|---|---|---|
| milky | vegetable | flavor | off flavor | mouth [symbol] | texture |
| milk | pea/ | vanilla | paper/ | acidic | tacky |
| | vegetable [symbol] | | cardboard | | |
| whey | [symbol] | caramel | [symbol] | bitter | [symbol] |
| [symbol] milk | vegetable | | chemical | salty | creamy |
| [symbol] | fresh walnut | | [symbol] | [symbol] /drying | dessert cream |
| baby milk | | | | | consistency |
| yoghurt | potato | | metallic | sweet | thick |
| butter | | | | spicy | watery |
| | | | | | airy foam |
| | | | | | coating |
| | | | | | sandy |
| | | | | | powdery [symbol] |
| | | | | | smooth |

[symbol]

indicates data missing or illegible when filed

Data Processing

The statistical processing method suited to this type of data is multiple factor analysis (J. Pagès, 1994) on the notes of the products. The set of descriptors generated by a judge is a group of variables. The graphs presented summarize all of the results provided by this method.

The statistical processing was performed with the software R version 2.14.1 (2011-12-22).

Results

Representation of the results in graph form is presented in FIG. 6.

Two families are distinguished on dimension 1 the two pea protein isolates according to the invention/the two pea proteins, and with dimension 2 the four samples may be characterized according to their texture, odor and taste.

Regarding the texture, the ready to drink beverages with PISANE® and NUTRALYS® S85F are thicker than those with the pea protein isolates according to the invention.

Regarding the odor, the beverage with the pea protein isolate No. 1 according to the invention is more vanilla than the pea protein isolate No. 2, whereas with PISANE®, the odor is more pea.

Regarding the taste, PISANE® appears spicy and chemical and, as for the odor, more pea, walnut and vegetable. The ready to drink beverages with PISANE and NUTRALYS® S85F have in common the bitter and paper-cardboard nature.

As regards the beverages with the pea protein isolates in accordance with the invention, beverage No. 1 (with isolate No. 1) is more milky and vanilla and beverage No. 2 is more cereal and milk jam/caramel.

### Example 8: Replacement of Milk Protein a Protein Isolates in UHT-Treated Dessert Creams for Clinical Nutrition

The nutritive formulations based on milk, pea and competing pea protein and pea protein isolates according to the invention are presented in the following table (substitution of the order of 23%):

| | | Nutritional | | Nutritional |
|---|---|---|---|---|
| | | formulation No. 2 | Nutritional | formulation with |
| | Control | according to the | formulation with | competing pea |
| Ingredients | formulation | invention | pea protein | protein |
| Demineralized water | 64.30 | 64.30 | 64.30 | 64.30 |
| Sucrose | 12.40 | 12.40 | 12.40 | 12.40 |
| Milk protein Isolate | 12.00 | 9.60 | 9.60 | 9.60 |
| (MPI-Ingredia) | | | | |
| [symbol] seed oil | 3.94 | 3.94 | 3.94 | 3.94 |
| Modified corn starch | 3.50 | 3.50 | 3.50 | 3.50 |
| CLEARAM [symbol] | | | | |
| CR3020 | | | | |
| (ROQUETTE | | | | |
| FRERES) | | | | |
| Maltodextrin | 1.70 | 1.70 | 1.70 | 1.70 |
| GLUCIDEX [symbol] | | | | |
| IT19 | | | | |
| (ROQUETTE | | | | |
| FRERES) | | | | |
| Corn dextrin | 1.50 | 1.50 | 1.50 | 1.50 |
| NUTRIOSE [symbol] | | | | |
| FM06 | | | | |
| Milk flavoring | 0.36 | 0.36 | 0.36 | 0.36 |
| Soybean [symbol] | 0.30 | 0.30 | 0.30 | 0.30 |
| Pea protein isolate | | 2.40 | | |
| No. 2 (according to | | | | |
| the invention- [symbol] | | | | |
| example 1 above) | | | | |
| Pea protein | | | | |
| NUTRALYS [symbol] | | | | |
| S85F(ROQUETTE | | | | |
| FRERES) | | | | |
| Pea protein | | | | 2.40 |
| PISANE [symbol] | | | | |
| (COSUCRA) | | | | |
| Total | 100 | 100 | 100 | 100 |

The amounts being indicated as weight percentages. [symbol]
indicates data missing or illegible when filed

The nutritional values per 100 g are as follows:

| | | Nutritional | Nutritional | Nutritional |
|---|---|---|---|---|
| | | formulation | formulation | formulation |
| | | No. 2 according | with | with |
| | Control | to the | pea | competing |
| | formulation | invention | protein | pea protein |
| [symbol] energy | 151 | 153 | 153 | 153 |
| (kCal) | | | | |
| Protein | 10.3 | 10.1 | 10.1 | 10.1 |
| content | | | | |
| (g) | | | | |
| Of which | 10.3 | 8.1 | 8.1 | 8.1 |
| milk protein | | | | |
| Of which | 0 | 0 | 0 | 0 |
| pea protein | | | | |
| isolates | | | | |
| Of which | 0 | 2 | 2 | 2 |
| pea protein | | | | |
| Fat ( [symbol] ) | 4.7 | 4.9 | 4.9 | 4.9 |
| Carbohydrates | 17.8 | 17.7 | 17.7 | 17.7 |
| (g) | | | | |
| Of which | 12.9 | 12.8 | 12.8 | 12.8 |
| sucrose | | | | |
| Fiber (g) | 1.2 | 1.3 | 1.3 | 1.3 |
| Of which | 1.2 | 1.3 | 1.3 | 1.3 |
| soluble | | | | |
| Of which | 0 | 0 | 0 | 0 |
| insoluble | | | | |

[symbol]

indicates data missing or illegible when filed

The process for manufacturing the beverages is as follows:
Preheat the water to 50° C.,
Dry-mix all the powders (milk protein, pea protein, pea protein isolates, maltodextrins, dextrins, sucrose and starch),
Add the powder mix to the water at 50° C., disperse with a whip for 1 minute and then mix with a SILVERSON blender at 3000 rpm for 30 minutes at 50° C.,
Add the flavoring to said solution,
Place the lecithin and the oil in a separate mixing container, stir and heat to 50° C.,
After 30 minutes of hydration, add the lecithin and the off mixture to the main batch, using a shear of 10 000 rpm for 5 minutes,
Sterilize the product at 133° C. for 55 seconds in a tubular exchanger and then package at 70° C.,
Store at 4° C.

Comparison of the Sensory Properties of Dessert Creams for Clinical Nutrition.

The panel is qualified for tasting formulated products. It received training so as to check its performance in terms of:
Capacity to discriminate the products
Consensus, correct use of the descriptors
Repeatability, ability to detect a product submitted twice

The panel consisted of 26 people, among the Roquette staff, and on the day of tasting, 11 people were present, among whom six were specifically trained on the subject of dessert creams.

The products were prepared and then stored in a refrigerator.

They were served to the panelists at room temperature.

Tasting Conditions
In the sensory analysis laboratory: individual tasting cubicles, white walls, calm environment (to facilitate concentration)
White light (to have exactly the same vision of the product)
At the end of the morning or the afternoon (to be at the height of the sensory capacities)
Products rendered anonymous with a three-figure code (to prevent the code from influencing the assessment of the products)
Products presented in a random order (to prevent order and persistence effects)

Exercise

The method employed to compare the products was the Flash Profile (J. M. Sieffermann, 2000).

The products are all presented simultaneously. It is a matter of comparing the products by making a succession of classifications: the panelists choose the descriptors which appear to them to be the most pertinent to discriminate between the products, and classify the products according to these descriptors; it is possible that several products are grouped in the same row.

### Example

Sensory descriptor: Fondant

Here is the list of descriptors presented to the panelists as a guide:

DESSERT CREAMS

| | | | |
|---|---|---|---|
| Descriptor | Definition | Procedure | Reference |

APPEARANCE

| | | | |
|---|---|---|---|
| Glossy | which reflects light | explore the product visually | Raw egg yolk |
| | (not glossy/ | | |
| | very glossy) | | |
| Granular | which contains particles | explore the product visually | Brown sugar |
| (appearance) | (numerous, and/or | | |
| | large-sized) | | |
| | (not granular/very granblar) | | |

TASTE

| | | | |
|---|---|---|---|
| Sweet | [symbol] taste generated by a | Taste the product | Castor sugar |
| | sucrose solution | | |
| Bitter | [symbol] taste which gives a sour | Taste the product | Caffeine/coffee |
| | unpleasant sensation, with | | |
| | a caffeine solution | | |
| Pea | has a pea taste | Tasto the product | Pea |

TEXTURE
spoonful

| | | | |
|---|---|---|---|
| Thick | resists flowing | evaluate the stress resistance of the | Honey |
| (spoon) | (not thick/very thick) | product by turning the spoon in the jar | |
| Short | forms a thin flow teickle/falls | Take up a product unit with a spoon. | SojaSun |
| | in blobs | Raise and turn the spoon over. Check the | |
| | (long/short texture) | length of the flow trickle | |

Mouthfeel
Inital phase (perception when first placed in the mouth)

| | | | |
|---|---|---|---|
| Thick (in | flows with difficulty in the mouth | put a product unit in the mouth and move | [symbol] |
| the mouth) | (not thick/very thick) | it around the oral cavity | |

Chewing phase (perception during chewing)

| | | | |
|---|---|---|---|
| Fondant | Evaluation of the dissolvability of the | keeep moving the product around in the | Ice cream |
| | product in the mouth by the action | mouth and evaluate the time taken to | |
| | of the saliva. | dissolve it. | |
| | (not fondant/very fondant) | | |
| Creamy | has a soft contact and melts in the mouth | put a product unit in the mouth and | Thick creme |
| | (not creamy/very creamy) | checkwhether it gives a soft contact | [symbol] |
| | | and whether it lines the oral cavity | |
| Granular | contains particles | slide the tongue against the palate | Brown sugar |
| (in the | (smooth/very granular) | and evaluate the perception of | |
| mouth) | | small grains in the mouth | |

Residual phase (changes arising during chewing)

| | | | |
|---|---|---|---|
| Tacky | adheres to the walls of the oral cavity | place a sample on the tongue, press it | Soft caramel |
| | (not tacky/very tacky) | against the palate and evaluate the force | |
| | | required to remove it with the tongue | |

[symbol]

indicates data missing or illegible when filed

Data Processing

The statistical processing method suited to this type of data is multiple factor analysis (J. Pagès, 1994) on the date-rows of the products. In order for the results to be clearer, the MFA was performed several times; globally, and per criterion (aspect, odor, taste, texture). The graphs presented summarize all of the results provided by this method.

The statistical processing was performed with the software R version 2.14.1 (2011-12-22).

Results

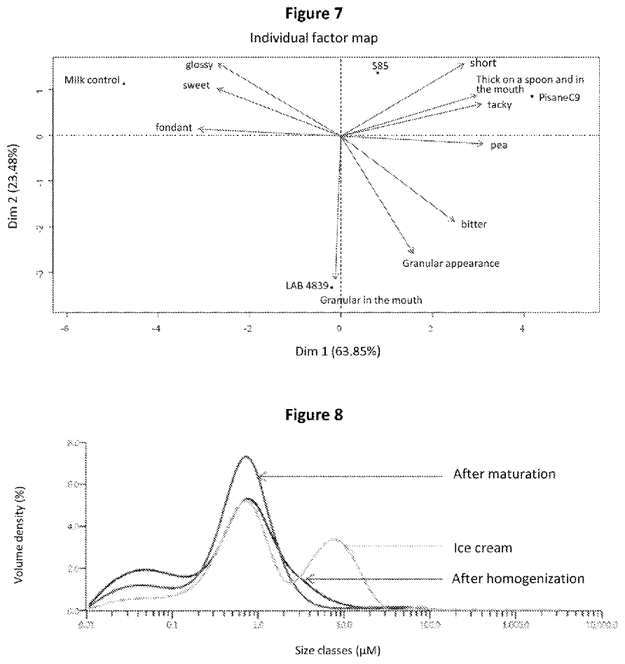

As is seen in FIG. 7, the dessert creams are consensually discriminated by all the panelists with a very high dimension 1 at almost 64% which describes the extreme products in the following manner.

The milk control has the glossiest appearance and melts in the mouth, but is the least thick and has the sweetest taste.

Regarding the texture, the dessert creams with PISANE® C9 and NUTRALYS® S85F are thicker than those with the pea protein isolate according to the invention.

Regarding the taste, the dessert cream with the pea protein isolate is less pea than the test with PISANE® CS and NUTRALYS® S85F.

### Example 9. Replacement of Milk Protein with the Pea Protein Isolate in Ice Creams/Iced Desserts

Four recipes are developed:
Control with 100% milk protein
Recipe No. 1: 50% milk protein replaced with the pea protein NUTRALYS® S85F;
Recipe No. 2: 50% milk protein replaced with the pea protein isolate No. 1, according to the invention;
Recipe No. 3: 50% milk protein replaced with the pea protein isolate No. 2 according to the invention;

| | | Recipe | Recipe | Recipe |
|---|---|---|---|---|
| | Control | No. 1 | No. 2 | No. 3 |
| Water | 48.70 | 48.77 | 48.79 | 48.79 |
| Sucrose | 13.00 | 13.00 | 13.00 | 13.00 |
| Cream (35% fat) | 27.40 | 27.40 | 27.40 | 27.20 |
| Lactose | 0.00 | 2.10 | 2.10 | 2.10 |
| Skimmed milk powder | 5.80 | 2.05 | 2.05 | 2.05 |
| 70/81 Glucose syrup | 4.00 | 4.00 | 4.00 | 4.00 |
| Pea protein | 0.00 | 1.58 | 0.00 | 0.00 |
| NUTRALYS [symbol] S85F | | | | |
| Pea protein isolate | 0.00 | 0.00 | 1.56 | 0.00 |
| No. 1 according | | | | |
| to the invention | | | | |
| Pea protein isolate | 0.00 | 0.00 | 0.00 | 1.56 |
| No. 2 according | | | | |
| to the invention | | | | |
| Stabilizers | 0.60 | 0.60 | 0.60 | 0.60 |
| (CREMODAN SE30) | | | | |
| Vanilla flavoring | 0.50 | 0.50 | 0.50 | 0.50 |
| IFF 10836706 | | | | |
| TOTAL | 100.00 | 100.00 | 100.00 | 100.00 |

The amounts being indicated as weight percentages. [symbol]
indicates data missing or illegible when filed

Nutritional values per 100 g

| | | | | |
|---|---|---|---|---|
| Energy (kCal) | 181.75 | 174.63 | 174.47 | 168.31 |
| Fat (g) | 10.03 | 10.15 | 10.15 | 10.15 |
| Carbohydrates (g) | 20.33 | 20.30 | 20.33 | 20.30 |
| Of which sugars (g) | 19.59 | 17.54 | 17.55 | 17.54 |
| Proteins (g) | 2.53 | 2.50 | 2.50 | 2.50 |
| Of which milk protein (g) | 2.53 | 1.25 | 1.25 | 1.25 |
| Of which plant protein (g) | 0.00 | 1.25 | 1.25 | 1.25 |
| Fiber (g) | 0.14 | 0.14 | 0.14 | 0.14 |
| Solids (%) | 33.98 | 33.85 | 33.86 | 33.87 |
| Lactose (%) | 4.09 | 4.06 | 4.06 | 4.06 |
| ESDL (%) | 3.84 | 3.95 | 3.95 | 3.95 |
| Degree of substitution of | 0 | 50 | 50 | 50 |
| the milk protein (%) | | | | |

The manufacturing process is as follows:
Add the skimmed milk to the container (40/45° C.),
Add the powdered ingredients to the container and stir for 15 minutes at 80 Hz in a CHOCOTEC batch cooker,
Mix together the stabilizers and the sugar, then Incorporate the mixture into the container,
Mix for 20 minutes at 80 Hz,
Incorporate the cream and the glucose syrup,
Mix for 15 minutes at 80 Hz,
Pasteurize at 80° C. for 3 minutes,
Cool to 70° C.—Half of the mixture obtained is homogenized directly; the other half is cooled to 50° C. When the first batch is homogenized, the second batch is heated to 70° C. and then homogenized,
Homogenize at 200 bar,
Cool in the maturation container to 4° C. and add the flavoring,
Leave to mature for 23 hours,
Beat to obtain an overrun of 95-100% and freeze at −30° C. to 1 hour,
Store the ice cream at −20° C.

Analyses

Characterization of the Mixtures During the Manufacturing Process

| | | Recipe | Recipe | Recipe |
|---|---|---|---|---|
| | Control | No. 1 | No. 2 | No. 3 |
| pH before maturation | 6.72 | 7.00 | [symbol] | 6.90 |
| | (0.5° C.) | (1° C.) | | (0.6° C.) |
| PH after maturation | 6.73 | 7.07 | 6.82 | 1.01 |
| | (−1.5° C.) | (0.1° C.) | (−2.4° C.) | (−2° C.) |
| Overrun of the mixture | 103 | 111 | 97 | 98 |
| (%) | | | | |
| Outlet temperature (° C.) | −5.4 | −5.8 | −6 | −5.6 |

[symbol]

indicates data missing or illegible when filed

It is noted that the expandability of the preparations made with the pea protein isolates in accordance with the invention is identical to that of the control and is not significantly different from that made with pea protein.

Viscosity Measurements

Viscosity (Pa [symbol] s)

| | Maturation | 10 s − 1 | 100 s − 1 |
|---|---|---|---|
| Control | Before | 0.24 | 0.098 |
| | After | 0.23 | 0.095 |
| Recipe No. 1 | Before | 0.36 | 0.18 |
| | After | 0.35 | 0.2 |
| Recipe No. 2 | Before | 0.18 | 0.10 |
| | After | 0.18 | 0.10 |
| Recipe No. 3 | Before | 0.2 | 0.11 |
| | After | 0.16 | 0.098 |

[symbol]

indicates data missing or illegible when filed

The recipe with pea protein shows the highest viscosities. The recipes with pea protein isolate in accordance with the invention are equivalent to the control recipe.

Particle Size Analysis

The particle size analysis was performed at various steps in the preparation of the ice cream for the purpose of evaluating the emulsifying capacity and the stability of the emulsion:
Size distribution of the fat globules after the homogenization step,
Size distribution of the fat globules after the maturation step,
Size distribution of the fat globules of the ice cream (equivalent to the size distribution of the fat globules after the beating step).

These analyses were also performed with addition of 0.1% SDS so as to determine whether the emulsion was created by aggregation/flocculation or by coalescence.

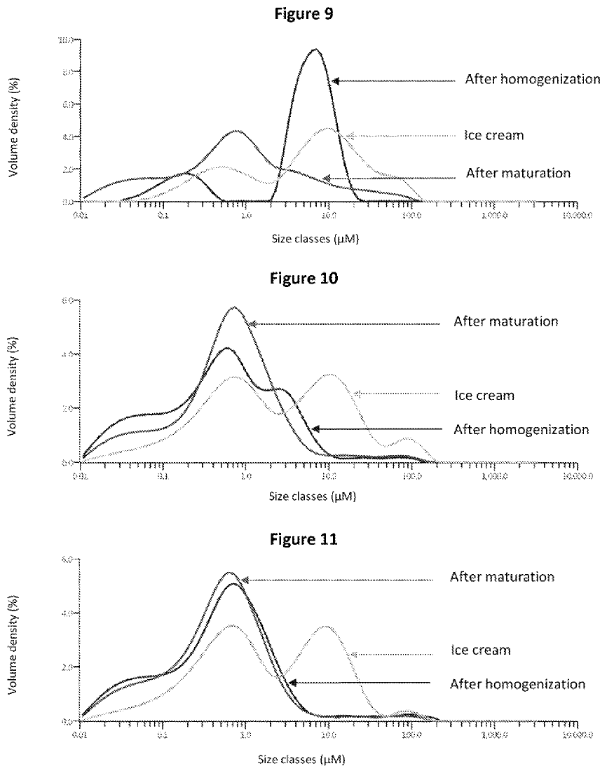

The results are presented in FIGS. 8 to 11.

For each recipe, the particle size distribution tends to decrease or to become more monomodal after maturation.

This change is very perceptible for the recipe with the pea protein NUTRALYS® S65F. This shows that the pea protein NUTRALYS® S85F is the slowest emulsifier for migrating at the interface of the fat globules.

In contrast, recipe No. 3 (with the pea protein isolate No. 2 in accordance with the invention) is just as good an emulsifier as the recipe containing 100% milk protein.

The pea protein isolate No. 1 in accordance with the invention is less emulsifying than the pea protein isolate No. 2 in accordance with the invention after homogenization, but has a tendency to become just as good after maturation.

### Example 10: Total Substitution of Milk Protein for the Pea Protein Isolates in Ice Creams/Iced Desserts

Three recipes were developed for these vegan ice creams:
Control: 100% pea protein NUTRALYS® S85F.
Recipe 1: 100% pea protein isolate No. 1 according to the invention,
Recipe 2: 100% pea protein isolate No. 2 according to the invention

| | Control | Recipe 1 | Recipe 2 |
|---|---|---|---|
| Water | 64.25 | 64.25 | 64.25 |
| Sucrose | 12.00 | 12.00 | 12.00 |
| Hydrogenated coconut oil | 8.00 | 8.00 | 8.00 |
| Glucose syrup Roquette 4280 | 11.50 | 11.50 | 11.50 |
| NUTRALYS [symbol] S85F | 3.50 | 0.00 | 0.00 |
| Stabilizers | 0.25 | 0.25 | 0.25 |
| ( [symbol] SE30) | | | |
| Vanilla flavoring | 0.50 | 0.50 | 0.50 |
| IFF 10836706 | | | |
| Pea protein isolate No. 1 | 0.00 | 3.50 | 0.00 |
| according to | | | |
| the invention | | | |
| Pea protein isolate No. 2 | 0.00 | 0.00 | 3.50 |
| according to | | | |
| the Invention | | | |
| Total | 100% | 100% | 100% |

The amounts being indicated as weight percentages. [symbol]
indicates data missing or illegible when filed

The nutritional values (per 100 g) are as follows:

| | Control | Recipe 1 | Recipe 2 |
|---|---|---|---|
| Energy value (kcal) | 172 | 172 | 172 |
| Total fat | 8.5 | 8.5 | 8.5 |
| of which saturated fat | 7.6 | 7.6 | 7.6 |
| Carbohydrates, | 21.2 | 21.2 | 21.2 |
| without fiber | | | |
| of which sugars | 14.6 | 14.6 | 14.6 |
| Fiber | 0.1 | 0.1 | 0.1 |
| Protein | 2.8 | 2.8 | 2.8 |
| Salt (sodium × 2.5) | 0.11 | 0.14 | 0.14 |
| Solids | 33.1 | 33.2 | 33.2 |

The manufacturing process is as follows:
Heat the water to 45° C.,
Mix the ingredients,
Mix the stabilizers with the sucrose,
Add the water and mix for 20 minutes,
Introduce the fat (melted coconut oil) and mix,
Pasteurize at 80° C. for 3 minutes,
Cool to 70° C.
Homogenize the mixture at 200 bar (in two stages)—30% in the 2^{nd} stage,
Add the flavoring,
Leave to mature with stirring at 4° C. for 20 minutes,
Beat to between 90-100% and cool at −30° C. for 1 hour,
Store at −18° C.

Analyses

Measurement of the Overrun Power (Iced Desserts)
Weight of an empty crucible of given volume V,
Measured mass=m_{c} in which m_{c} is the mass of the empty crucible
Weight of a crucible of given volume V, filled to the brim with the mixture before overrun
Measured mass=mc+mₘᵢₓ in which mₘᵢₓ is the mass of mixture corresponding to the volume V.
Weight of a crucible of given volume V, filled to the brim with the mixture after overrun (taken from the freezer)
Measured mass=mc+m_{ice} in which m_{ice} is the mass of ice (overrun mixture taken from the freezer) corresponding to the volume V.

The overrun measurement is then given by the formula:

$$Overrun = \frac{( {m}_{mix} - {m}_{ice} )}{{m}_{ice}} \times 100$$

Characterization of the Preparation Process

| | Control | Recipe 1 | Recipe 2 |
|---|---|---|---|
| pH after maturation | 7.25 | 6.84 | 6.89 |
| Density of the mixture (g/ml) | 1.05 | 1.06 | 1.07 |
| Overrun power (%) | 89 | 80 | 101 |
| Temperature on leaving | −5 | −4.8 | −4.8 |
| the freezer | | | |

Viscosity Measurement
Measurements taken at 4° C.
Rheometer: Physica MCR 301 Anton Paar
Geometry: concentric cylinder CC27
Nominal value: 0 to 200 s⁻¹ in 5 minutes

| Before maturation | Viscosity (mPa · s) | | |
|---|---|---|---|
| Reference | 10 s⁻¹ | 100 s⁻¹ | 200 s⁻¹ |
| Control | 131 | 60 | 50 |
| Recipe 1 | 23 | 51 | 40 |
| Recipe 2 | 22 | 50 | 39 |

| After maturation | Viscosity (mPa · s) | | |
|---|---|---|---|
| Reference | 10 s⁻¹ | 100 s⁻¹ | 200 s⁻¹ |
| Control | 120 | 65 | 56 |
| Recipe 1 | 76 | 50 | 48 |
| Recipe 2 | 74 | 50 | 44 |

It is thus noted that the viscosity is tower when the recipe comprises pea protein isolates according to the invention.

Measurement of the Texture
Measurement temperature: on leaving the freezer,
Rheometer: INSTRON 9506 machine:
Geometry: conical
Nominal value: imposed deformation up to 20 minutes,

| Freezer storage time | Hardness (N) | | |
|---|---|---|---|
| before measurement | 7 days | 14 days | 30 days |
| Control | 38.4 | 42.7 | 60.5 |
| Recipe 1 | 126.2 | 128.4 | 137 |
| Recipe 2 | 76 | 67.9 | 63 |

It is found that the hardness is globally better for the recipes with the pea protein isolates according to the invention. More particularly, the pea protein isolate No. 2 according to the invention has a remarkably high hardness, no doubt in relation to its higher overrun power (101%).

Measurement of the Size of the Emulsion of the Mixture and of the Iced Des Protocol:
MALVERN 3000 liquid route particle size analyzer (granulometer) (the solvent is demineralized water)
Optical model: 1.46+0.001i with a stirring speed of 1900 rpm.

The mixtures before and after maturation are characterized with and without SDS:
Without SDS: the sample is introduced directly into the particle size analyzer beaker containing only water,
With SDS: 0.1%, i.e. 0.6 g, of SDS is introduced directly into the beaker of the particle size analyzer After dissolution of the SDS, the sample is added for analysis.

The final ice cream is introduced unthawed into the bowl of the particle size analyzer. After melting and dispersing the ice cream, the measurement is taken.

The size of the emulsion, before and after maturation, with and without SDS, is given in the following table.

| Before maturation | Dx (10) | Dx (50) | Dx (90) | Dmode | D (4.3) |
|---|---|---|---|---|---|
| without SDS | μm | μm | μm | μm | μm |
| Control | 0.073 | 0.377 | 3.380 | 0.429 | 1.410 |
| Recipe 1 | 0.693 | 10.300 | 22.200 | 13.000 | 11.400 |
| Recipe 2 | 0.520 | 8.400 | 18.200 | 10.000 | 9.310 |

Without SDS, the emulsion of the mixture containing pea protein (control) has a smaller particle size than the emulsions prepared from the pea protein isolates according to the invention.

| Before maturation | Dx (10) | Dx (50) | Dx (90) | Dmode | D (4.3) |
|---|---|---|---|---|---|
| without SDS | μm | μm | μm | μm | μm |
| Control | 0.103 | 0.340 | 1.010 | 0.389 | 0.698 |
| Recipe 1 | 0.065 | 0.605 | 5.110 | 0.621 | 1.650 |
| Recipe 2 | 0.115 | 0.503 | 1.830 | 0.564 | 0.913 |

With SDS, the fat agglomerates are dispersed, and the Dmode is thus closer for the three tests. It should be noted that the formulation with the pea protein isolate No. 1 according to the invention has a particle size analysis peak with larger particles.

| After maturation | Dx (10) | Dx (50) | Dx (90) | Dmode | D (4.3) |
|---|---|---|---|---|---|
| without SDS | μm | μm | μm | μm | μm |
| Control | 0.085 | 0.379 | 2.690 | 0.433 | 1.210 |
| Recipe 1 | 0.102 | 5.300 | 16.200 | 8.470 | 6.780 |
| Recipe 2 | 0.088 | 4.460 | 15.400 | 7.820 | 6.150 |

| After maturation | Dx (10) | Dx (50) | Dx (90) | Dmode | D (4.3) |
|---|---|---|---|---|---|
| without SDS | μm | μm | μm | μm | μm |
| Control | 0.103 | 0.329 | 0.894 | 0.381 | 0.658 |
| Recipe 1 | 0.039 | 0.509 | 4.830 | 0.616 | 1.620 |
| Recipe 2 | 0.096 | 0.504 | 2.070 | 0.573 | 1.110 |

No major change is observed after maturation. The formulations with the pea protein isolates according to the invention are more polydisperse than the formulation with the pea proteins.

The emulsion size of the ice cream, in unmodified form, is measured without the presence of SDS.

| | Dx (10) | Dx (50) | Dx (90) | Dmode | D (4.3) |
|---|---|---|---|---|---|
| Without SDS | μm | μm | μm | μm | μm |
| Control | 0.092 | 0.333 | 3.950 | 0.352 | 2.390 |
| Recipe 1 | 0.131 | 0.776 | 15.900 | 0.531 | 5.820 |
| Recipe 2 | 0.178 | 0.814 | 51.400 | 0.518 | 14.500 |

The size of the major peak (Dmode) is similar for the three ice creams. However, the formulations with the pea protein isolates according to the invention are more polydisperse, especially with the isolate No. 2.

A comparative study was performed with commercial ice creams, which show that these ice creams contain an even larger number of coarse particles than the control recipes and recipes 1 and 2 in relation to their high content of fat globules.

Measurement of the Melting Behavior

Protocol:

Empirically, samples of iced desserts of a given volume are placed on a grille above a beaker. The following are then measured:
The time after which the first drop falls into the beaker,
The percentage of ice cream melted over time, over 3 hours.

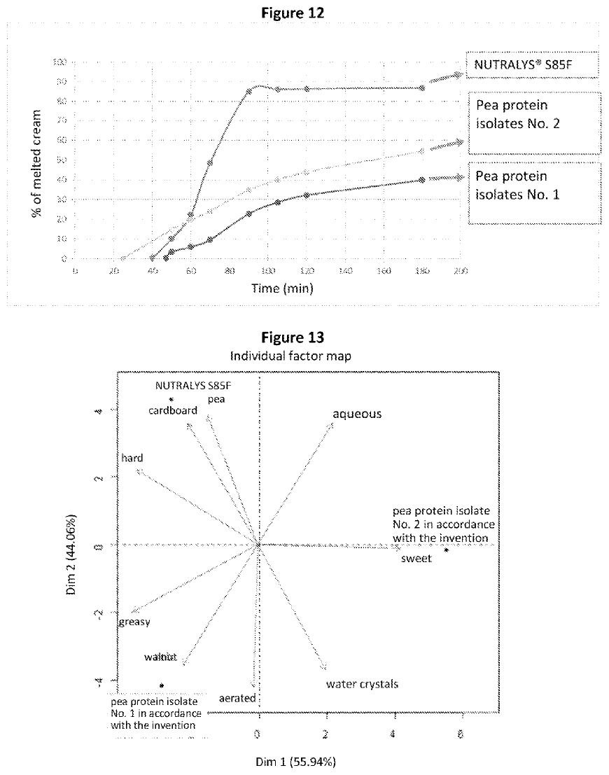

FIG. 12 clearly illustrates the fact that the melting is lesser for the ice creams prepared with the pea protein isolates according to the invention.

Sensory Analysis

The panel consisted of 15 people.

The panel, as in the preceding examples, is qualified for tasting products formulated with pea protein. It received training so as to check its performance in terms of:
Capacity to discriminate the products
Consensus, correct use of the descriptors
Repeatability, ability to detect a product submitted twice.

When compared with the ice creams prepared with pea protein, those of the invention are less bitter, have less of a pea taste and are less colored.

The iced desserts with the pea protein isolates No. 1 according to the invention have a few ice crystals and a more pronounced vanilla taste, are sweeter, and fatter than the other products.

The iced desserts with the pea protein isolates No. 2 according to the invention are sweet and fatty, and more creamy. They have a slightly more pronounced “green tea” taste.

Conclusion

During the process of manufacturing the iced desserts, the pea protein isolates according to the invention lead to a lower viscosity in comparison with pes protein.

The texture of isolate No. 1 is harder, but is not perceived by the panelists.

The two isolates lead above all to lowering the melting of the corresponding iced desserts.

In terms of taste, the best perception is for the iced desserts prepared with isolate No. 1, of sweet taste and pronounced flavor, less bitterness and less “pea” taste.

### Example 11: Comparison of the Sensory Properties of Ice Creams

The panel consisted of 20 people.

The panel is qualified for tasting products formulated with pea protein. It received training so as to check its performance in terms of:
Capacity to discriminate the products
Consensus, correct use of the descriptors
Repeatability, ability to detect a product submitted twice

Specifically, it received training in the correct use of the sensory descriptors of taste and texture, for instance:

| | | | |
|---|---|---|---|
| Descriptor | Definition | Procedure | Reference |

Flavors

| | | | |
|---|---|---|---|
| Pea taste | Typical pea taste. | Taste the product. | Pea |
| Sweet | Elementary taste | Taste the product. | Caster sugar |
| | generated | | |
| | by a sucrose solution | | |

Mouthfeel

| | | | |
|---|---|---|---|
| Hard | Evaluation of the force | Press a product | Confectionery |
| | required to obtain | unit between the | of cooked |
| | deformation | incisors. | sugar type |
| | or rupture of the product. | | |
| | (not hard/very hard) | | |
| Aerated | Evaluation of the | Explore visually | Chocolate |
| | amount of air bubbles | and by touch by | mousse |
| | trapped and visible. | placing a finger | |
| | (not aerated/very | parallel to the | |
| | aerated) | surface state and | |
| | | exert different | |
| | | pressures from | |
| | | top to bottom. | |
| Aqueous | Evaluation of the surface | Taste a product | Watermelon |
| | texture properly | unit and evaluate | |
| | qualifying the perception | the amount of | |
| | of the amount of water | water perceived | |
| | released by a product. | in the mouth. | |
| | (not aqueous/very | | |
| | aqueous) | | |
| Water | Evaluation of the | Rub the tongue | Recrystallized |
| crystals | presence of water | against the | ice |
| | crystals (no crystals/ | palate and | |
| | many crystals) | check whether | |
| | | the product | |
| | | contains crystals | |
| Greasy | Evaluation of the greasy | After swallowing | Olive oil |
| | film after swallowing. | a product unit, | |
| | (not greasy/very greasy) | evaluate the | |
| | | presence or | |
| | | absence of a | |
| | | greasy film on | |
| | | the palate or on | |
| | | the teeth by | |
| | | sweeping over | |
| | | their surface | |
| | | with the tongue. | |

The method also allows them to make comments on other descriptors that were not anticipated in this list.

Products

The ice creams are recipes No. 1, No. 2 and No. 3 those of Example 9.

Tasting Conditions:
In a sensory analysis laboratory: individual tasting cubicles, white walls, calm environment (to facilitate concentration).
White light (to have exactly the same vision of the product)
At the end of the morning of the afternoon (to be at the height of the sensory capacities)
Products rendered anonymous with a three-figure code (to prevent the code from influencing the assessment of the products)
Products presented in a random order (to prevent order and persistence effects)

Exercise

The method employed to compare the products was the Flash Profile (J. M. Sieffermann, 2000).

The products are all presented simultaneously. It is a matter of comparing the products by making a succession of classifications: the panelists choose the descriptors which appear to them to be the most pertinent to discriminate between the products, and classify the products according to these descriptors; it is possible that several products are grouped in the same row.

### Example

Sensory descriptor: Crunchy

Data Processing

The statistical processing method suited to this type of data is multiple factor analysis (J. Pagès, 1994) on the data-rows of the products in order for the results to be clearer, the MFA was performed several times; globally, and per criterion (aspect, odor, taste, texture). The graphs presented summarize all of the results provided by this method.

The analyses were performed using the R software (on open sale):
R version 2.14.1 (2011-12-22)
Copyright (C) 2011 The R Foundation for Statistical Computing
ISBN 3-900051-07-0
20 Platform: 1386-pc-mingw32/1386 (32-bit)

The software is a working environment which requires the loading of modules containing the calculation functions such as the FactoMineR version 1.19 package.

Results

The results are shown in FIG. 13.

The three samples are all evaluated in terms of creamy texture, cold and fondant and in terms of pea, vanilla and bitter taste.

However, a few descriptors allow them to be differentiated:
The ice cream with NUTRALYS® S85F appears harder with a pea and cardboard taste.
The one with pea protein isolate No. 1 in accordance with the invention is more greasy and aerated with a walnut note.
The one with pea protein isolate No. 2 in accordance with the invention is judged to be sweeter.

### Example 12: Use of the Pea Protein Isolates in “Non-Dairy Coffee Creamer/Whitener” Matrices

a. 100% Substitution for Sodium Caseinates

The object here is to substitute 100% of the sodium caseinates and to obtain a product that is stable in coffee.

Measurement of the viscosity of the emulsions after pasteurization and measurement of the stability in coffee make it possible to illustrate the improvement in the functional properties of the pea protein isolates relative to NUTRALYS® in their ability to substitute for sodium caseinates.

The recipes developed are as follows:

| | | Recipe 2 | Recipe 3 | |
|---|---|---|---|---|
| | | 100% | 100% | |
| | | pea | protein | |
| | | protein | isolate | |
| | | isolate | No. 2 | |
| | Recipe 1 (Control | No. 1 | according | |
| | Recipe) | according | to the | Recipe 4 |
| | 100% sodium | to the | invention | 100% |
| | caseinate | invention | (60% | NUTRALYS ® |
| | (60% solids) | (60% solids) | solids) | S85F |
| | % | % | % | (60% solids) |
| Glucose syrup 3072 | 45.85 | 45.85 | 45.85 | 45.85 |
| (Roquette FRERES) | | | | |
| Hydrogenated coconut oil 32- | 23.36 | 23.36 | 23.36 | 23.36 |
| 34 (Dislab) | | | | |
| NUTRALYS ® S85F batch | | | | 1.75 |
| WB67J | | | | |
| Pea protein isolate No. 1 | | 1.75 | | |
| according to the Invention | | | | |
| Pea protein isolate No. 2 | | | 1.75 | |
| according to the invention | | | | |
| Sodium caseinate EM7 | 1.75 | | | |
| (DMV) | | | | |
| K₂HPO₄_E340 (Merck) | 1.46 | 1.46 | 1.46 | 1.46 |
| Dimodan HP_E471 (Danisco) | 0.58 | 0.58 | 0.58 | 0.58 |
| Water | 27.00 | 27.00 | 27.00 | 27.00 |
| TOTAL | 100.00 | 100.00 | 100.00 | 100.00 |

The amounts being indicated as weight percentages.

The manufacturing process is as follows:
Melt the fat at 80° with constant stirring,
Add the Dimodan HP to the melted fat to dissolve the monoglycerides,
Heat 90% of the water to 50° C. and add the proteins. Hydrate with constant stirring for 30 minutes,
Dissolve the phosphate salts in the residual water at 40° C.,
After 30 minutes of hydration, add the glucose syrup and the phosphate salts to the main mixture.
Pre-emulsify the mixture of fat/Dimodan HP in the main mixture for 5 minutes at 10 000 rpm,
Place the product at 75° C. in a Niro Panda 2K Soavi (GEA) high-pressure homogenizer at a pressure of 160 bar in the first stage, and 30 bar in the second stage,
Pasteurize at 80° C. for a few seconds and then place the product in cold water to stop the heat treatment.

The analyses performed on the formulation are as follows:

1. Viscosity Analysis

The viscosity measurements on the concentrated emulsions after the heat treatment step are performed at 65° C., the usual atomization temperature.

Apparatus:
Physica MCR 301 Anton Pear rheometer
Geometry CC27
Method 0 to 1000 s⁻¹ 660 s

The results obtained on the various recipes are as follows:

| | Viscosity (mPa · s) | | | | |
|---|---|---|---|---|---|
| | 5s-1 | 10s-1 | 40s-1 | 100s-1 | 1000s-1 |
| Control recipe | 89 | 69 | 47 | 44 | 42 |
| Recipe 2 | 77 | 59 | 40 | 34 | 24 |
| Recipe 3 | 77 | 60 | 42 | 36 | 26 |
| Recipe 4 | 775 | 530 | 270 | 197 | 85 |

The viscosities of the emulsions of recipes 2 and 3 after pasteurization are closer to the milk control than that of recipe 4 prepared with pea protein, which makes it possible to dry a low-viscosity emulsion with a high solids content as here at 60% by weight.

2. Solubility as a Function of the pH

| | | Pea protein | Pea protein | Sodium |
|---|---|---|---|---|
| | | isolate No. 1 | isolate No. 2 | caseinate |
| | NUTRALYS | according to the | according to the | EM7 |
| | S85F | invention | invention | (DMV) |
| pH 3 | 50.3 | 53.3 | 58.9 | 82.0 |
| pH 4 | 15.2 | 39.7 | 38.6 | 7.0 |
| PH 5 | 11.3 | 37.7 | 36.8 | 9.0 |
| pH 6 | 21.2 | 50.3 | 53.9 | 94.0 |
| pH 7 | 36.8 | 54.8 | 59.9 | 94.0 |
| pH 8 | 55.1 | 57.4 | 62.4 | 94.0 |

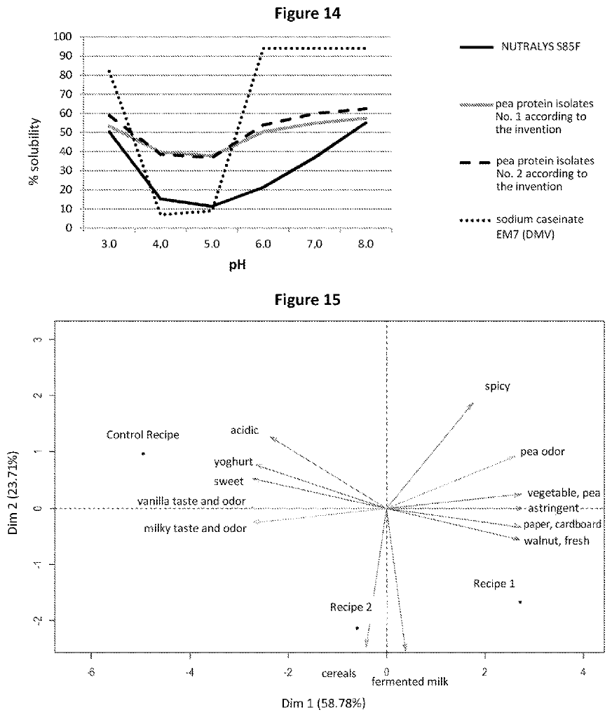

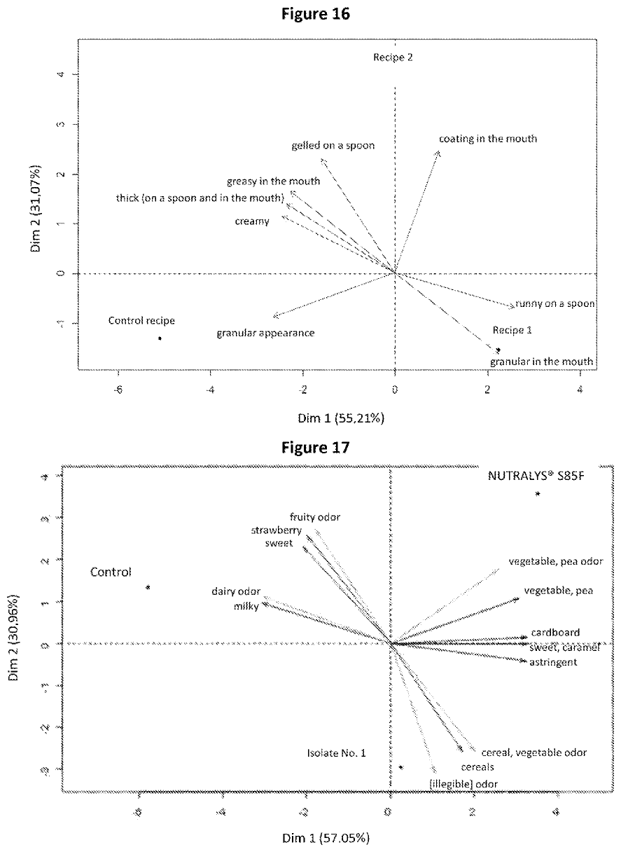

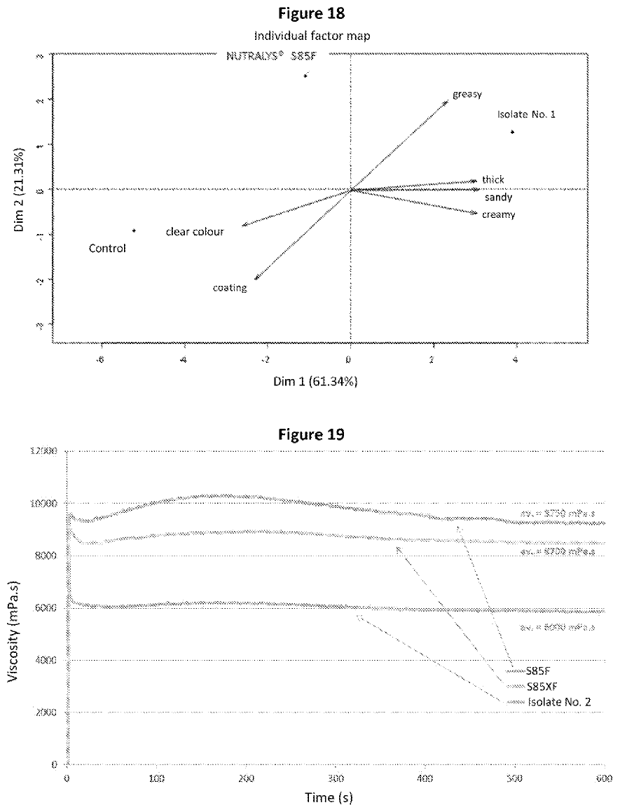

FIG. 14 illustrates the change in solubility of the pea protein isolates according to the Invention relative to caseinate, as a function of the pit, and reflects their excellent behavior.

### Evaluation of the Stability in Coffee

Reconstitution of Coffee
Weigh out 2 g of soluble coffee:
heat drinking water (calcium content of 136 mg and magnesium content of 60 mg) at 80° C. and add 135 g of said water to the 2 g,
add 12.7 g of concentrated emulsion to the coffee.

The flocculation in the coffee appears to be less substantial with the recipes containing the pea protein isolates according to the invention, relative to that obtained with pea protein. However, this may be correlated with the improvement in solubility of said isolates relative to pea protein.

a. 50% Substitution for Sodium Caseinates

The object here is to substitute 50% of the sodium caseinates and to obtain a product that is stable in coffee.

Measurement of the viscosity of the emulsions after pasteurization and measurement of the stability in coffee make it possible to illustrate the improvement in the functional properties of the pea protein isolates relative to NUTRALYS® in their ability to substitute for sodium caseinates.

The recipes developed are as follows:

| | Recipe 1 | Recipe 2 | Recipe 3 |
|---|---|---|---|
| | (Control | 50% pea protein | 50% |
| | Recipe) | isolate No. 2 | NUTRALYS ® |
| | 100% | according to the | S85F + 50% |
| | sodium | invention + 50% | sodium |
| | caseinate | sodium caseinate | caseinate |
| | (60% solids) | (60% solids) | (60% solids) |
| | % | % | % |
| Glucose syrup 3072 | 45.85 | 45.85 | 45.85 |
| (Roquette FRERES) | | | |
| Hydrogenated coconut | 23.36 | 23.36 | 23.36 |
| oil 32-34 (Dislab) | | | |
| NUTRALYS ® S85F | | | 0.87 |
| batch WB67J | | | |
| Pea protein isolate | | 0.87 | |
| No. 2 according | | | |
| to the invention | | | |
| Sodium caseinate EM7 | 1.75 | 0.88 | 0.88 |
| (DMV) | | | |
| Joha@ | 1.46 | 1.46 | 1.46 |
| KM2_E339_E452_E331 | | | |
| Dimodan HP_E471 | 0.58 | 0.58 | 0.58 |
| (Danisco) | | | |
| Water | 27.00 | 27.00 | 27.00 |
| TOTAL | 100.00 | 100.00 | 100.00 |

The amounts being indicated as weight percentages.

The nutritional values per 100 g are as follows:

| Recipe No. | Recipe 1 | Recipes 2 and 3 |
|---|---|---|
| Moisture content (%) | 40 | 40 |
| Calorific energy (kCal) | 369 | 369 |
| per 100 g | | |
| Fat (g) | 24.0 | 24.0 |
| Protein conten (g) | 1.6 | 1.5 |
| Of which milk protein | 1.6 | 0.8 |
| Pea protein isolates | 0 | 0.7 |
| Carbohydrates (g) | 33.0 | 33.0 |
| Of which sugars | 5.3 | 5.3 |

The manufacturing process is as follows:
Melt the fat at 80° with constant stirring,
Dissolve the monoglycerides and diglycerides in the liquid oil,
Dissolve the powdered protein in water at 50° C. over 30 minutes,
Add the glucose syrup and the phosphate salts already dissolved in part of the water,
Pre-emulsify the melted fat in the aqueous solution by stirring at 10 000 rpm,
Pasteurize at 80° C. for a few seconds,
Place the product at 75° C. in a Niro Panda 2K Soavi (GEA) high-pressure homogenizer at a pressure of 160 bar in the first stage, and 30 bar in the second stage,
Dilute the mixture to 50% solids to atomize at 180° C. (Tᵢₙₗₑₜ) and 90° C. (Tₒᵤₜₗₑₜ) in the device with an evaporation capacity of 10 to 12 l/h.

The analyses performed on the formulation are as follows:

1) pH of the Emulsion

| pH | Recipe 1 | Recipe 2 | Recipe 3 |
|---|---|---|---|
| On the emulsion at | 7.69 | 9.24 | 8.72 |
| 25° C. | | | |
| On coffee at 75° C. | 6.45 | 6.35 | 6.10 |

2) Capacity of the Emulsion

Measurement of the size of the lipid globules (with a laser particle size analyzer) makes it possible to determine the capacity of the pea protein isolates according to the invention to form lipid globules of the smallest possible size.

| | Dx(10) | Dx(50) | Dx(90) | D (4.3) | Mode |
|---|---|---|---|---|---|
| | (μm) | (μm) | (μm) | (μm) | (μm) |
| Recipe 1 | 0.281 | 0.577 | 1.23 | 0.681 | 0.551 |
| Recipe 2 | 0.289 | 0.563 | 1.10 | 0.668 | 0.558 |
| Recipe 3 | 0.279 | 0.564 | 1.23 | 1.15 | 0.534 |

These results clearly show that the 50/50 mixture has a particle size distribution similar to the 100% caseinate control.

3) Viscosity of the Emulsions Containing 60% Solids at 65° C. (Before Atomization)

Apparatus:
Physica MCR 301 Anton Paar rheometer
Geometry: CC27
Method 0 to 1000 s⁻¹ in 660 s

| | Viscosity (mPa.s) | | | |
|---|---|---|---|---|
| | 5 s [symbol] | 10 s [symbol] | 40 s [symbol] | 100 s [symbol] |
| Recipe 1 | 89 | 69 | 47 | 44 |
| Recipe 2 | 49 | 48 | 43 | 39 |
| Recipe 3 | 170 | 132 | 89 | 77 |

[symbol]

indicates data missing or illegible when filed

The lowest viscosity of the 50/50 mixture makes it possible to atomize at a solids content higher than that conventionally required for caseinates.

4) Stabilization of Powdered “Non-Dairy Coffee Creamer” in Coffee

Reconstitution of Coffee:
a. Weigh out 2 g of soluble coffee
b. Add 8 g of emulsion and 150 ml of drinking water (calcium content of 136 mg and magnesium content of 60 mg) at 80° C.

The stability of the emulsion in coffee is determined by measuring the color variation of the preparation—color measurement according to the L (white balance), a (yellow balance) and b (green balance) coordinates, the white color in coffee being one of the key criteria sought by manufacturers and consumers.

A difference of 2 points for the measurement of the L parameter of the coffees prepared with the 50/50 mixture (L=+96) with regard to the control coffees prepared with caseinates (L=+98) reflects the excellent stability of the mixture with the pea protein isolates in accordance with the invention.

### Example 13. Use of the Pea Protein Isolates for the Preparation of Stirred Yoghurts

The object here is to replace 30% of the milk protein.

The recipes developed are as follows:

| | | Recipe | Recipe with pea | Recipe |
|---|---|---|---|---|
| | | with | protein isolate No. | with |
| | | pea | 1 in accordance | pea |
| | Control | protein | with the invention | protein. |
| In % | recipe | Recipe 1 | Recipe 2 | Recipe 3 |
| Reconstituted | 88.80 | 74.50 | 74.50 | 74.50 |
| skimmed milk | | | | |
| Cream | 2.75 | 2.42 | 2.42 | 2.42 |
| (Les fayes 35% fat) | | | | |
| Sugar | 6.00 | 6.00 | 6.00 | 6.00 |
| Modified starch | 1.50 | 3.20 | 3.20 | 3.20 |
| CLEARAM ® | | | | |
| OR 4015 from | | | | |
| Roquette Fréres | | | | |
| NUTRALYS ® S85F | | 1.40 | | |
| NUTRALYS ®F85F | | | | 1:40 |
| Pea protein isolate | | | 1.40 | |
| No. 1 according to | | | | |
| the Invention | | | | |
| PROMILK 852 | 0.77 | | | |
| A Ingredia | | | | |
| Pectin OM 020 | 0.18 | 0.16 | 0.16 | 0.16 |
| HERBSTREITH & FOX | | | | |
| Water | | 12.32 | 12.32 | 12.32 |
| Total | 100.00 | 100.00 | 100.00 | 100 |

The amounts being indicated as weight percentages:

| | | | | |
|---|---|---|---|---|
| Solids | 17.73 | 18.28 | 18.31 | 18.28 |
| Total protein | 3.70 | 3.70 | 3.70 | 3.70 |
| Dairy protein | 3.70 | 2.58 | 2.58 | 2.58 |
| Plant protein | 0.00 | 1.12 | 1.12 | 1:12 |
| Lipids | 1.01 | 1.01 | 1.03 | 1.01 |
| Carbohydrates | 12.30 | 12.99 | 12.99 | 12.99 |
| of which sugars | 10.98 | 10.14 | 10.14 | 10.14 |
| Kcal/100 g | 73.10 | 75.84 | 75.86 | 75.84 |
| Degree of substitution | 0.00 | 30.19 | 30.19 | 30.19 |

The manufacturing process is as follows:
heat water to 60° C.,
add the proteins and have to hydrate for 1 hour,
add the cream while mixing with a POLYTRON homogenizer for 2 minutes,
add the sugar/starch mixture over 10 to 15 minutes,
homogenize at high pressure (two stages: 1^{st} stage 180 bar—2^{nd} stage 200 bar) at 75-80° C.,
pasteurize with a Power Point International tubular exchanger at 95° C., 6 minutes—20 l/h,
add the ferments (YoFlex® YF-L812—60 U/250 L),
acidity at 42° C. to PH 4.6 (acidification time of 6-6 hours),
stir at 3600 rpm and at 42° C.,
smooth at 37/38° C. at 3600 rpm with a Spindle 2G
a place in a pot and store at 4° C.

Viscosity Measurement

| | Temperature measurement: |
|---|---|
| | 13° C. |
| Rheometer: | Physica MOR 301 Anton Paar |
| Geometry: | CC27 |
| Method: | 0 to 350 s⁻¹ in 180 s and |
| | return from 350 s⁻¹ to 0 in 180 s |

The values are given to within ±5%.

| | D + 3 | | | | | |
|---|---|---|---|---|---|---|
| | | | | | | Hysteresis |
| | Viscosity (mPa · s) | | | | | area |
| Reference | 5 s⁻¹ | 10 s⁻¹ | 40 s⁻¹ | 100 s⁻¹ | 350 s⁻¹ | (Pa) |
| Recipe 2 | 4.3 | 2.6 | 1.03 | 0.53 | 0.2 | 4130 |
| Recipe 1 | 3.9 | 2.38 | 0.98 | 0.52 | 0.21 | 3335 |
| Recipe 3 | 3.76 | 2.3 | 0.97 | 0.53 | 0.22 | 2810 |
| Control recipe | 3.12 | 2.1 | 0.79 | 0.4 | 0.15 | 2420 |

| | D + 7 | | | | | |
|---|---|---|---|---|---|---|
| | | | | | | Hysteresis |
| | Viscosity (mPa · s) | | | | | area |
| Reference | 5 s⁻¹ | 10 s⁻¹ | 40 s−1 | 100 s⁻¹ | 350 s⁻¹ | (Pa) |
| Recipe 2 | 4.12 | 2.49 | 1.02 | 0.535 | 0.212 | 3710 |
| Recipe 1 | 3.26 | 2.21 | 0.84 | 0.41 | 0.16 | 2551 |
| Recipe 3 | 3.71 | 2.29 | 0.96 | 0.52 | 0.21 | 2905 |
| Control recipe | 4.17 | 2.51 | 1.02 | 0.53 | 0.2 | 4200 |

| | D + 14 | | | | | |
|---|---|---|---|---|---|---|
| | | | | | | Hysteresis |
| | Viscosity (mPa · s) | | | | | area |
| Reference | 5 s⁻¹ | 10 s⁻¹ | 40 s⁻¹ | 100 s⁻¹ | 350 s⁻¹ | (Pa) |
| Recipe 2 | 3.93 | 2.38 | 0.99 | 0.524 | 0.21 | 3260 |
| Recipe 1 | 3.54 | 2.17 | 0.94 | 0.52 | 0.22 | 2420 |
| Recipe 3 | 4.13 | 2.51 | 1.02 | 0.51 | 0.19 | 4860 |
| Control recipe | 2.74 | 1.82 | 0.7 | 0.35 | 0.138 | 2200 |

Recipe 3 has the closest behavior to the control recipe but with, however, inversion of the viscosity curve relative to the change in viscosity of the control recipe at D+7 and D+14.

Specifically, recipe 3 regains in viscosity at D+14, and is the most resistant to shear at D+14.

Recipe 1 is more viscous and resistant to shear than recipe 3 at D+7, but this reverses from D+14.

Recipe 2 with the pea protein isolate in accordance with the invention is the most viscous of the four recipes, and is more viscous than the control recipe. Its viscosity decreases over time.

These results demonstrate that, by virtue of its behavior, the pea protein isolate in accordance with the invention would make it possible to decrease the amount of starch in this recipe if it is desired to make it resemble the viscosity of the control recipe.

The same goes, but to a lesser extent, for recipes 1 and 3.

### Example 14: Comparison of the Sensory Properties of Stirred Yoghurts

For the taste evaluation, the panel consisted of 11 people. For the texture evaluation, the panel consisted of 12 people.

The panels are qualified for tasting products formulated with pea protein. They received training so as to check their performance in terms of:
Capacity to discriminate the products
Consensus, correct use of the descriptors
Repeatability, ability to detect a product submitted twice

Specifically, they received training in the correct use of the sensory descriptors of taste and texture, for instance:

Taste Descriptors:

| flavor/odor | | | | |
|---|---|---|---|---|
| milky | vegetable | flavor | off flavor | taste/mouthfeel |
| milk | pea/vegetable AG | vanilla | paper/ | acidic |
| | | | cardboard | |
| whey | cereals | caramel | detergent | bitter |
| fermented | vegetable | | chemical | salty |
| milk | | | | |
| reconstituted | fresh walnut | | glue | astringent/drying |
| baby | | | | |
| milk | | | | |
| yoghurt | potato | | metallic | sweet |
| butter | | | | spicy |

Texture Descriptors

| | | | |
|---|---|---|---|
| Descripton | Definition | Procedure | Reference |

Appearance

| | | | |
|---|---|---|---|
| Glossy | Glossy or lustrous | Explore the prodcut visually. | Raw egg yolk |
| | appearance resulting from | | |
| | the tendency of a surface to | | |
| | reflect light. | | |
| | (not glossy/very glossy) | | |
| Granular | Evaluation of the granular | Explore the prodcut visually. | Brown sugar |
| | nature and of the number | | |
| | and size of particles of a | | |
| | product | | |
| | (not granular/very granular) | | |

Texture on a spoon

| | | | |
|---|---|---|---|
| Gelled | Evaluation of the consistency. | Explore the prodcut visually. | Gelatin |
| | of the product. | | |
| | (not gelled/very gelled) | | |
| Thick | Evaluation of the consistency | Take up a product unit with a | Honey |
| | of the product. | spoon, Raise and turn the spoon | |
| | (not thick/very thick) | over. Check the flowability. | |
| Runny | Evaluation the capacity to | Apply the spoon perpendicular to | Water |
| | trickle without dividing into | the surface, place under pressure | |
| | drops. | and gently withdraw it vertically. | |
| | (not runny/very runny) | | |
| Coating | Evaluation of the capacity to | Apply the spoon perpendicular to | Custard |
| | form a coat on the back of | the surface and gently withdraw it | |
| | the spoon. | vertically. | |
| | (not coating/very coating) | | |

Mouthfeel

| | | | |
|---|---|---|---|
| Aqueous | Evaluation of the texture | Taste a product unit and evaluate | Watermelon |
| | property of the surface | the amount of water perceived in | |
| | qualifying the perception of | the mouth. | |
| | the amount of water | | |
| | released by a product. | | |
| | (not aqueous/very aqueous) | | |
| Drying | Evaluate the texture | Chew a product unit and check | Cranberry juice |
| | property describing the | whether the inside of the mouth | |
| | perception of the absorption | becomes dry. | |
| | of moisture by the product. | | |
| | (not drying/very drying) | | |
| Greasy | Evaluation the greasy film | After swallowing a product unit, | Olive oil |
| | after swallowing. | evaluate the presence of absence | |
| | (not greasy/very greasy | of a greasy flim on the palate or | |
| | | teeth by sweeping over their | |
| | | surface with the tongue | |
| Creamy | Evaluation of the soft and | Chew a product unit and check | Créme fraiche, |
| | fondant texture the product. | whether it causes a soft contact. | double cream |
| | (not creamy/very creamy) | and whether it lines the mouth, | |
| Thick | Evaluation of the ease of the | Rub the tongue against the palate | sweet and |
| | product to flow in the | and check whether the product | concentrated |
| | mouth. | flows easily. | milk; boney |
| | (not thick/very thick) | | |
| Tacky | Evaluation of the force | After chewing a few times | Soft caramel |
| | required to detach products | successively, press the product | |
| | that adhere to the inside of | between the teeth and measure | |
| | the oral cavity. | the force required for it to be | |
| | (not tacky/very tacky) | detached from the teeth | |
| Granular | Evaluation of the granular | Rub the tongue against the palate | Brown sugar |
| | nature and of the number | and check whether the product | |
| | and size of particles of a | contains particles. | |
| | product. | | |
| | (not granular/very granular) | | |

Products

The three products tested of example 11 (control recipe, recipe 1 and recipe 2) were evaluated three days after being produced and were presented at a temperature of about 10° C. (products stored in a refrigerator, evaluated when taken out).

Tasting Conditions
In a sensory analysis laboratory: individual tasting cubicles, white walls, calm environment (to facilitate concentration).
White light (to have exactly the same vision of the product)
At the end of the morning or the afternoon (to be at the height of the sensory capacities)
Products rendered anonymous with a three-figure code (to prevent the code from influencing the assessment of the products)
Products presented in a random order (to prevent order and persistence effects)

Exercise

The method employed to compare the products was the Flash Profile (J. M. Sieffermann, 2000)

The products are all presented simultaneously. It is a matter of comparing the products by making a succession of classifications: the panelists choose the descriptors which appear to them to be the most pertinent to discriminate between the products, and classify the products according to these descriptors; it is possible that several products are grouped in the same row.

### Example

Sensory descriptor: Crunchy

Two lists of descriptors, relating to the taste or to the texture, were proposed to the panelists as a guide: they are attached in the appendix of this report.

Data Processing

The statistical processing method suited to this type of data is multiple factor analysis (J. Pages, 1994) on the data-rows of the products. In order for the results to be clearer, the MFA was performed several times; globally, and per criterion (aspect, odor, taste, texture). The graphs presented summarize all of the results provided by this method.

The statistical processing was performed with the software R version 2.14.1 (2011-12-22).

Results:

The results are presented in FIGS. 15 (taste) and 16 (texture):
The stirred yoghurt containing NUTRALYS® S85F has a funny and granular texture in the mouth accompanied by a pea, cardboard, fresh walnut taste;
The yoghurt with milk protein appears more fatty and creamy, thick with a granular aspect, its taste is more typical of yoghurt, sweet and milky;
The yoghurt with the pea protein isolate No. 1 in accordance with the invention lies between the control and the tests with NUTRALYS® S85F, and stands out by having a cereal and fermented milk taste and also a particularly coating texture in the mouth.

### Example 15. Use of the Pea Protein Isolates for the Preparation of Strawberry-Flavored Dairy Beverages

The object here is to replace 50% of the milk protein.

The recipes developed are as follows:

| | | 50% | |
|---|---|---|---|
| | | Substitution | |
| | | with | |
| | | the pea | |
| | | protein | |
| | | isolate | 50% |
| | 100% | No. 1 | Substitution |
| | control | according | with |
| | Milk | to the | NUTRALYS ® |
| | protein | invention | S85F |
| The amounts being given | % | % | % |
| as weight percentages | | | |
| UHT skimmed | 86.7 | 56.1 | 56.1 |
| Water | | 28.3 | 28.3 |
| Sucrose | 5.80 | 7.20 | 7.20 |
| Cream (35% fat) METRO | 4.70 | 4.20 | 4.20 |
| Milk protein concentrate:4892 | 1.05 | | |
| (Fonterra) | | | |
| NUTRALYS ® S85F | | | 2.40 |
| batch WB67J | | | |
| Pea protein solate No. 1 | | 2.40 | |
| according to the invention | | | |
| Strawberry purée (Metro) | 1.35 | 1.35 | 1.35 |
| Modified starch CLEARAM ® | 0.40 | 0.40 | 0.40 |
| CH 3020- Roguette Fréres | | | |
| Strawberry flavoring | 0.005 | 0.005 | 0.005 |
| (M _0051126) | | | |
| Colorant E124 7 luted tenfold) | 0.013 | 0.019 | 0.013 |
| TOTAL | 100.00 | 100.00 | 100.00 |

The manufacturing process is as follows:
heat milk and water to 50° C.,
add the protein to the mixture,
hydrate for 1 hour at 50° C. with stirring,
preheat the fat to 50° C. after 45 minutes,
add the sugar and starch to the main preparation,
add the colorant, the flavoring and the strawberry puree to the main preparation,
mix for 5 minutes,
pre-emulsify the fat in the main preparation for 5 minutes at 10 000 rpm,
homogenize the product online at 65° C.—190 bar (two stages)
sterilize the product at 30 liters/hour with a residence time of 7 seconds at 138° C. on a tubular exchanger
cool to 40° C. and store at +4° C.

### Example 16: Comparison of the Sensory Properties of Strawberry Flavored Daily Beverages

For the taste evaluation: the panel consisted of 12 people.

The panels are qualified for tasting products formulated with pea protein. They received training so as to check their performance in terms of:
Capacity to discriminate the products
Consensus, correct use of the descriptors
Repeatability, ability to detect a product submitted twice

Specifically, they received training in the correct use of the sensory descriptors of taste and texture, for instance:

List of Descriptors:

| | | | |
|---|---|---|---|
| Descripton | Definition | Procedure | Reference |

Appearance

| | | | |
|---|---|---|---|
| Smooth | absence of heterogeneous | Explore the product | Liquid matrix: |
| | particles at the surface of the | visually. | pure water |
| | product | | Powder matrix: |
| | (not smooth/very smooth) | | icing sugar |
| Glossy | Glossy or lustrous appearance | Explore the product | Raw egg yolk |
| | resulting from the tendency of a | visually. | |
| | surface to reflect light. | | |
| | (not glossy/very glossy) | | |

Mouthfeel

| | | | |
|---|---|---|---|
| Thick | Evaluation of the ease of the | Rub the tongue against the | Sweetened |
| | product flow in the mouth. | palate and check whether | concentrated milk, |
| | (not thick/very thick) | the product flows easily; | honey |
| Aqueous | Evaluation of the surface | Taste a product unit and | Watermelon |
| | texture property qualifying the | evaluate the amount of | |
| | perception of the amount of | water percieved in the | |
| | water released by a product | mouth. | |
| | (not aqueous/very aqueous) | | |
| Creamy | Evaluation of the soft and | Chew a product unit and | Créme fraiche, |
| | fondant texture of the product. | check whether it causes a | thick cream |
| | (not creamy/very creamy) | soft contact and whether it | |
| | | lines the mouth. | |
| Sandy | Evaluation of the granular | Chew a product unit until it | Certain pears |
| | nature and of the number of | is ready to swallow and | |
| | particles of a product. | evaluate the perception of | |
| | (not sandy/very sandy) | small grains in the mouth. | |

Odor and Taste

| flavor/odor | | | | |
|---|---|---|---|---|
| milky | vegetable | flavor | off flavor | taste/mouth feel |
| milk | pea/vegetable GA | vanilla | paper/cardboard | acidic |
| whey | cereals | strawberry | detergent | bitter |
| fermented milk | vegetable | | chemical | salty |
| reconstituted baby milk | fresh walnut | | glue | astringent/drying |
| yoghurt | potato | | metallic | sweet |
| butter | | | | spicy |

Tasting Conditions
In a sensory analysis laboratory: individual tasting cubicles, white walls, calm environment (to facilitate concentration)
White light (to have exactly the same vision of the product)
At the end of the morning or the afternoon (to be at the height of the sensory capacities)
Products rendered anonymous with a three-figure code (to prevent the code from influencing the assessment of the products)
Products presented in a random order (to prevent order and persistence effects)

Exercise

The method employed to compare the products was the Flash Profile (J. M. Sieffermann, 2000).

The products are all presented simultaneously. It is a matter of comparing the products by making a succession of classifications: the panelists choose the descriptors which appear to them to be the most pertinent to discriminate between the products, and classify the products according to these descriptors; it is possible that several products are grouped in the same row.

### Example

Sensory descriptor: Crunchy

Two lists of descriptors, relating to the taste or to the texture, were proposed to the panelists as a guide: they are attached in the appendix of this report.

Data Processing

The statistical processing method suited to this type of data is multiple factor analysis (J. Pagés, 1994) on the data-rows of the products. In order for the results to be clearer, the MFA was performed several times; globally, and per criterion (aspect, odor, taste, texture). The graphs presented summarize all of the results provided by this method.

The statistical processing was performed with the software R version 2.14.1 (2011-12-22).

Results:

The results are presented in FIGS. 17 (taste) and 18 (aspect and texture in the mouth).

In terms of taste, the panelists clearly identified the control by qualifying it as more sweet, milky (odor and taste), and strawberry (odor and taste) than the tests formulated with pea protein.

The test with the pea protein isolate No. 1 according to the invention is qualified in odor and taste as vegetable cereal while maintaining a milky odor, whereas the test with NUTRALYS retains a vegetable pes odor and taste.

In terms of texture, all the products were judged to be aqueous. Their characterization is essentially made on dimension 1; two families are then distinguished:
the milk control, which is judged to be more coating in the mouth followed by the test with NUTRALYS®,
the pea protein isolate No. 1 according to the invention is judged to be more greasy.

### Example 17: Protein Enrichment of Biscuits (Intended for Specialized/Slimming or Sports Nutrition)

| | | | | Pea protein | Pea protein |
|---|---|---|---|---|---|
| | | NUTRALYS ®: | | isolate No. 1 | Isolate No. 2 |
| | | S85F | NUTRALYS ® | according to the | according to: |
| | Control | Roquette Fréres: | Pea-BF | invention | the invention |
| Water: | 6 | 7.4 | 6.7 | 6.5 | 6.5 |
| Sodium bicarbonate | 0.3 | 0.3 | 0.3 | 0.3 | 0.3 |
| Ammonium bicarbonate | 0.2 | 0.2 | 0.2 | 0.2 | 0.2 |
| Sucrose | 18 | 17.7 | 17.7 | 17.9 | 17.9 |
| Glucose Syrup 4779 | 2.5 | 2.5 | 2.5 | 2.5 | 2.5 |
| Roquette Fréres | | | | | |
| Fat Biscuitine 600 | 13 | 12.8 | 12.8 | 12.9 | 12.9 |
| Soybean lecithin | 0.2 | 0.2 | 0.2 | 0.2 | 0.2 |
| Wheat flour | 57.8 | 37.2 | 37.2 | 37.8 | 37.6 |
| Test proteins | 0 | 19.7 | 19.7 | 19.9 | 49.9 |
| Skimmed milk powder | 1.5 | 1.5 | 1.5 | 1.5 | 1.5 |
| Salt | 0.2 | 0.2 | 0.2 | 0.2 | 0.2 |
| Sodium pyrophosphate | 0.2 | 0.2 | 6.2 | 0.2 | 0.2 |
| Vanilla powder | 0.1 | 0.1 | 0.1 | 0.1 | 0.1 |
| | 100 | 100 | 100 | 100 | 100 |

The Amounts being Indicated as Weight Percentages.

The formulations have the following composition:

The nutritional values of these formulations are as follows:

| | | | | Pea protein isolate | Pea protein isolate |
|---|---|---|---|---|---|
| | | NUTRALYS ® S85F | NUTRALYS ® | No. 1 according to | No. 2 accoring to |
| | Control | Roquette Fréres | Pea-BF | the invention | the invention |
| Calories (kCal) | 462 | 472 | 472 | 472 | 472 |
| Proteins: | 7.9 | 23.2 | 23.2 | 23.2 | 23.2 |
| Fat | 15.7 | 17.4 | 17.4 | 17.4 | 17.4 |
| Carbohydrates | 72.3 | 55.8 | 55.8 | 55.8 | 55.8 |
| ofwhich.DP1,2. | 22.9 | 22.9 | 22.9 | 22.9 | 22.9 |
| Fiber | 1.3 | 1.1 | 1.1 | 1.1 | 1.1 |
| . . .insoluble | 1.3 | 0.9 | 0.9 | 0.9 | 0.9 |
| . . .soluble | 0.0 | 0.2 | 0.2 | 0.2 | 0.2 |
| kCal/protein (%) | 7 | 20 | 20 | 20 | 20 |

The manufacturing process is as follows:
Dissolve the sodium bicarbonate and the ammonium bicarbonate in water. Add the sugar and the glucose syrup and mix in a Hobart planetary mixer equipped with a flat paddle, for 1 minute at speed 1 to fully dissolve the sugars.
Add the fat and the lecithin and mix for 2 minutes at speed 2.
Add the rest of the powders in a single portion and mix for 2 minutes at speed 1 and then for 1 minute at speed 2.
Leave the dough to stand for 15 minutes to complete the hydration of the powders and the homogeneity of the preparation.
Place the dough in the hopper of the biscuit machine so that the dough is pressed in a mold cavity between two rollers so as to form the biscuits.
Recover the biscuits on a conveyor belt, then place them on a baking tray.
Bake in a MWE Econo type fan oven for 9 minutes at 170° C. (fan speed 2).

The analyses performed are as follows:

One of the first important criteria in the production of biscuits on a biscuit machine is the “machinability” of the dough.

An over-hydrated dough will be tacky and will not detach from the mold cavities.

A dough that is too dry will not fill the mold cavities and will form biscuits with anomalies.

Adding a large amount of protein has an impact on the dough texture. The table below illustrates the hydration adjustments necessary to compensate for the incorporation of various proteins into a biscuit dough.

Specifically, proteins with a more or less substantial affinity for water will bind part of the water of the formulation. This water will then no longer be available to “plasticize” the dough, which will then be too dry to be formed. Increasing the hydration of the dough will then be essential to correct this defect.

| | | | | | Pea protein |
|---|---|---|---|---|---|
| | | NUTRALYS ® | | Pea protein isolate | isolate No. 2 |
| | | S85F | NUTRALYS ® | No. 1 according to | according to the |
| | Control | Roquette Fréres | Pea-BF | the invention | invention. |
| Percentage increase in | 0 | 23.2 | 11.7 | 7.8 | 7.8 |
| water in the recipe for | | | | | |
| the dough to be | | | | | |
| workable | | | | | |

Unfortunately, in a dry biscuit (less than 3% water in the finished product), it is not desirable to add too much water since this will have an impact on the baking time and conditions.

Furthermore, adding more water will have an effect on the kinetics of concentration and of recrystallization of the sugars. Now, this last point is a determining factor for the texture, especially the crunchiness, of a biscuit.

Proteins that are soluble but sparingly functional such as the pea protein isolates of the present invention thus make it possible to limit this correction to only +8% added water as opposed to 12% for a non-functional and insoluble protein and more than 23% for a soluble and functional protein.

A rapid sensory analysis performed on the biscuits produced gave the following results.

| | NUTRALS ® | | Pea protein isolate | Pea protein isolate |
|---|---|---|---|---|
| | S85F | NUTRALYS ® | No. 1 according | No. 2 according to |
| Control | Roquette Fréres | Pea-BF | the Invention | the Invention |
| Reference in | Biscuits | Sandy and pasty | More crunchy | More crunchy |
| terms of lexture | sparingly | biscuits, Less | biscuits. Less pasty | biscuits. Texture |
| and taste | crunchy, very | crunchiness. Pea | texture, no real pea | less pasty than |
| | pasty in the | after-taste. | taste. | NUTRALYS ® BF |
| | mouth, Pea | | | |
| | after-taste. | | | |

### Example 18: Protein Enrichment of Chocolate Muffins (Intended for Specialized/Slimming or Sports Nutrition)

The formulations have the following composition:

| | NUTRALYS ® | NUTRALYS ® | |
|---|---|---|---|
| | SB5F | XF Roquette | Pea protein isolate No. 2 |
| | Roquette Fréres | Fréres | according to the invention |
| Whole wheat flour | 120.0 | 120.0 | 120.0 |
| SweetPearl ® P200 Roquette Fréres | 210.0 | 210.0 | 210.0 |
| Skimmed milk powder | 10.0 | 10.0 | 10.0 |
| Coconut powder (defatted) | 15.0 | 15.0 | 15.0 |
| NUTRISOFT ® 55 from Brenntag | 5.0 | 5.0 | 5.0 |
| Baking powder | 5 | 5 | 5 |
| Test proteins | 60.0 | 60.0 | 60.0 |
| NUTRALYS ® WF Roquette Fréres | 60.0 | 60.0 | 60.0 |
| WPC 515 from FONTERRA | 52.0 | 52.0 | 52.0 |
| Salt | 2.0 | 2.0 | 2.0 |
| Plant oil | 60.0 | 80.0 | 80.0 |
| NEOSORB ® 70/70 | 50.0 | 50.0 | 50.0 |
| Roquette Fréres | | | |
| Dark chocolate | 20.0 | 20.0 | 20.0 |
| Water | 160.0 | 160.0 | 160.0 |
| Vanilla extract | 10.0 | 10.0 | 10.0 |
| Whole egg | 140.0 | 140.0 | 140.0 |
| liquid | | | |
| | 1000.0 | 1000.0 | 1000.0 |

The amounts being indicated by weight (in grams).

The manufacturing process is as follows:
Heat mixture B to melt the chocolate
Mix the powders A in a Hobart planetary mixer equipped with a flat paddle, for 1 minute at speed 1
Add the melted mixture B to the powders and mix for 2 minutes at speed 1
Finally, add C and mix for 2 minutes at speed 1, Scrape the bowl and mix again for 2 minutes at speed 2
Spread the preparation in paper muffin molds (70 g per mold)
Bake in a MWE Econo type fan oven at 180° C. to 15 minutes (fan speed 2, air inlet closed).

The analyses performed are as follows:

Viscosity of the Muffin Batters:

The measurement is taken using an AR2000 rheometer from the company TA Instruments, with the following profile:
Time: 600 s
Speed: 160 rpm
Temperature: 25° C.

The results are shown in FIG. 19.

In the muffins, the viscosity of the preparation will have an impact on the rising during baking and thus on the final volume. The pea protein isolates according to the invention have a much lower viscosity than the other pea proteins.

### Example 19: Protein Enrichment of Griddle Cake Instant Mix (Intended for Specialized/Slimming or Sports Nutrition)

The object here is to replace 50% of the milk protein.

The formulations have the following composition:

| | | | Pea protein |
|---|---|---|---|
| | | | isolate No. 2 |
| | | | according |
| | NUTRALYS ® | NUTRALYS ® | to the |
| | S85F | S85F | invention |
| | % | % | % |
| Whole wheat flour | 14 | 14 | 14 |
| WPC 450 from | 12.5 | 6.2 | 6.2 |
| FONTERRA | | | |
| Protein | — | 6.3 | 6.3 |
| Wheat flour | 7.3 | 7.3 | 7.3 |
| Skimmed milk powder | 2 | 2 | 2 |
| Egg white powder | 1.6 | 1.6 | 1.6 |
| Powdered fat CEGEPAL | 1.4 | 1.4 | 1.4 |
| Baking powder | 0.8 | 0.8 | 0.8 |
| Salt | 0.4 | 0.4 | 0.4 |
| Water | 60 | 60 | 60 |
| | 100 | 100 | 100 |

The amounts being indicated as weight percentages.

The nutritional values of those formulations are as follows:

| | | | 50% WPC replaced with pea | |
|---|---|---|---|---|
| | CONTROL | | protein isolates | |
| | | Griddle | | Griddle |
| Per 100 g | Powder | cakes | Powder | cakes |
| Calories (kCal) | 375 | 211 | 373 | 211 |
| Protein (%) | 36.6 | 20.7 | 36.6 | 20.7 |
| Fat (%) | 7.1 | 4.0 | 7.2 | 4.0 |
| Carbohydrates | 41.1 | 23.2 | 40.6 | 23.0 |
| . . . of which sugars (%) | 2.5 | 1.4 | 2.0 | 1.4 |
| Dietary fiber (%) | 3.7 | 2.1 | 3.9 | 2.2 |
| kCal/protein (%) | 39 | 39 | 39 | 39 |

The manufacturing process is as follows:
Mix all the powders
Add the water and mix with a whip to obtain a homogeneous preparation
Leave to stand for 2 minutes
Cook in a pancake pan or multi-griddle cake skillet for about 2 minutes, turning the griddle cakes when half cooked

The analyses performed are as follows:

Impact on the Viscosity of the Preparations

The measurement is taken using an RVA rheometer, with the following profile:

| Time (s) | Speed (rpm) | Temperature (° C.) |
|---|---|---|
| 60 | 20 | 25 |
| 120 | 50 | 25 |
| 180 | 100 | 25 |

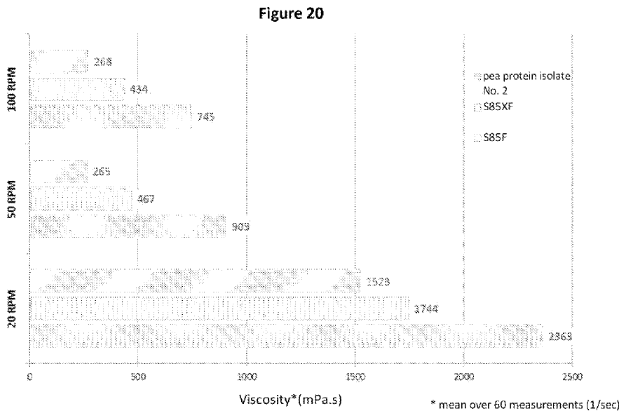

The results are shown in FIG. 20.

The RVA viscosity measurements on the protein-enriched preparations show that those with the pea protein isolate according to the invention are less viscous than those with the other pea proteins.

This influence on the viscosity has an impact on the rising of the griddle cakes during cooking.

A rapid sensory analysis performed on the griddle cakes produced gave the following results.

| | | Pea protein |
|---|---|---|
| Control: | | isolate No. 2 |
| NUTRALYS ® | NUTRALYS ® | according to |
| S85F | S85F | the invention |
| Liquid batter | More viscous batter | Liquid batter close |
| | | to the control |
| product appears drier | More of a “griddle cake” | Thickness close to the |
| in the mouth | style (thicker) | control |
| Appearance more like a | But with a pea taste | Milder taste and more |
| “crepe griddle cake” than | | supple texture |
| a “griddle cake” (thinner). | | |

| | |
|---|---|
| White crumb | More beige crumb |

### Example 20: Protein-Enriched Gluten-Free Bread

Traditional bread has a protein content of about 10%.

However, in gluten-free products, the protein content is very low. Protein supplementation of these products is then sought to re-equilibrate the nutritional values by means of gluten-free proteins such as pea protein.

The formulations have the following composition:

| | | | Pea protein of | |
|---|---|---|---|---|
| | MIX B SCHAR | | various origins | |
| | g | % | g | % |
| MIX B SCHAR | 600.0 | 49.1% | 430.0 | 42.2% |
| Pea protein | 0.0 | 0.0% | 70.0 | 6.9% |
| Dry yeast | 8.0 | 0.8% | 8.0 | 0.8% |
| Water | 500.0 | 49.1% | 500.0 | 49.1% |
| Canola oil | 6.0 | 0.6% | 6.0 | 0.6% |
| Salt | 5.0 | 0.5% | 5.0 | 0.5% |
| | 1019.0 | 100% | 1019.0 | 100% |

The nutritional values of these formulations are as follows:

| | MIX B | Bread enriched with pea | Traditional wheat |
|---|---|---|---|
| per 100 g | SCHAR | protein of various origins | flour bread |
| Calories (kCal) | 236 | 241 | 267 |
| Protein | 2.9 | 9.7 | 9.6 |
| Fat | 1.5 | 2.2 | 1.0 |
| Carbohydrates | 52.7 | 45.5 | 54.8 |
| . . . of which DP1,2 | 2.9 | 2.6 | 1.0 |
| Fiber | 2.4 | 2.1 | 1.8 |
| . . . insoluble fiber | 2.4 | 2.1 | 1.5 |
| . . . soluble fiber | 0.0 | 0.1 | 0.0 |
| kCal/protein (%) | 4.8 | 16.1 | 14.4 |

The analyses performed are ma follows:

Viscosity of the doughs obtained from pea protein of four different origins including the according to the invention):

| | | | | | Pea protein | Pea protein |
|---|---|---|---|---|---|---|
| | | | | | isolate No. 1 | isolate No. 2 |
| | MIX B | NUTRALYS ® | NUTRALYS ® | NUTRALYS ® | according to | according to the |
| | SCHAR | F85F | SB5F | PEA-BF | the invention | invention |
| Viscosity of | 0 | +++ | ++ | ++ | 0 | 0 |
| the dough | | | | | | |
| Color | White/nacre | Brown/orange | Brown/orange | Brown/orange | Brown/orange | Brown/orange |
| | | ++ | + | + | | |
| Maximum | 67 | 52 | 51 | 58 | 85 | 65 |
| height (mm) | | | | | | |
| Weight (g) | 345 | 353 | 362 | 357 | 354 | 352 |
| Percentage | 11% | 9% | 6% | 7% | 8% | 9% |
| water loss | | | | | | |
| during baking | | | | | | |
| Moisture | 54.2 | 55.0 | 54.8 | 55.0 | 54.5 | 54.7 |
| content of | | | | | | |
| the final | | | | | | |
| product (% | | | | | | |
| H2O) | | | | | | |

The SCHAR mix corresponds here to the control reference for a gluten-free bread. The results show that protein enrichment of this mix has an impact on the viscosity of the preparation and on the final volume (maximum height), except for the pea protein isolates obtained according to the invention, which do not effect either the viscosity of the preparation or the final volume.

### Example 21: Protein-Enriched Bread

The formulations have the following composition:

| | | Pea protein |
|---|---|---|
| | | isolate No. 2 |
| | NUTRALYS ® | according to the |
| | BF | invention |
| Wheat flour | 900 | 900 |
| Pea protein | 100 | 100 |
| Salt | 18 | 18 |
| Dry yeast | 7 | 7 |
| Ascorbic acid | 0.2 | 0.2 |
| NUTRILIFE ® AM17; | 0.2 | 0.2 |
| enzyme | | |
| Water (20° C.) | 725 | 725 |
| | 1750.4 | 1750.4 |

The amounts are indicated by weight (in grams).

The nutritional values of these formulations are as follows:

| | Traditional white | Bread rich in pea |
|---|---|---|
| | bread | protein |
| Calories (kCal/kJ) | 266 | 252 |
| Proteins | 9.8 | 14.3 |
| Fat | 1.0 | 1.3 |
| Carbohydrates | 54.8 | 45.9 |
| . . . of which DP 1,2 | 0.0 | 0.0 |
| Total fiber | 1.6 | 1.4 |
| . . . insoluble fiber | 1.6 | 1.3 |
| . . . soluble fiber | 0.0 | 0.1 |
| kCal/protein (%) | 14 | 23 |

The analyses performed are as follows:

| | NUTRALYS ® | Pea protein |
|---|---|---|
| | PEA-BF | isolate No. 2 |
| Volume (cm³) | 1505 | 1745 |
| Bread weight after baking (g) | 441.3 | 435.0 |
| Water loss during baking | 11.7% | 13.3% |
| Weight of three disks of breadcrumbs/ | 20.4 | 13.0 |
| 50 mm diameter (g) | | |
| Bread density (g/cm³) | 0.293 | 0.249 |
| Crumb density (g/cm³) | 0.346 | 0.221 |

The volume and density are in favor of the pea protein isolates according to the invention, which allows better rising and thus a more aerated and softer, less dense bread.

A rapid sensory analysis performed on the breads produced gave the following results.

| | |
|---|---|
| NUTRALYS ® BF | Pea protein isolate No. 2 according to the invention |
| Pea taste, dry crumb | Absence of pea taste; less intense “toast” taste; |
| | extra light crumb; large volume |

### Example 22: High-Protein Crisps (Protein Content>60%)

High-protein crisps are small cereals obtained by extrusion, with a protein content of greater than 60%.

These cereals are used as inclusion in cereal preparations such as cereal bars or clusters.

These high-protein crisps are occasionally the only solution for the protein enrichment of these cereal products since the incorporation of protein in powder form has an excessive impact on the texture of the finished product.

The technical difficulty of high-protein crisps lies in achieving protein contents of greater than 60%, or even 70%, while preserving the crunchiness.

The crunchiness of the extruded products is directly associated with the expansion. In cereals obtained by extrusion cooking, expansion takes place at the die outlet under pressure of water vapor.

The high-protein crisp formulations containing 75% protein have the following composition:

| | | | Pea protein |
|---|---|---|---|
| | | | isolate No. 1 |
| | | | according |
| | NUTRALYS ® | NUTRALYS ® | to the |
| | BF | S85F | invention |
| Pea protein | 88% | 88% | 88% |
| PREGEFLO ® C100: | 8% | 8% | 8% |
| pregelatinized | | | |
| waxy corn starch | | | |
| Corn starch | 4% | 4% | 4% |

The amounts being indicated as weight percentages.

The procedure is as follows:

The crisps, or extruded cereals were obtained on a CLEXTRAL Evolum 25 brand co-rotating twin-screw extruder equipped with a shearing screw profile.

To compare the tests, the parameters are set in a first stage so as to have only the “type of protein” variable.

The extrusion is performed as follows.

| | Fixed parameters |
|---|---|
| Screw speed (rpm) | 300 |
| Chopping speed (rpm) | 800 |
| Powder flow rate (kg/h) | 6 |
| Water flow rate (kg/h) | 1.4 |
| Die | Three holes (3 mm diameter), |
| | two of which are blocked |
| Temperatures of barrels 1 | 50-80-100-130-150-130 |
| to 6 (° C.) | |
| Knife blade | double |

The analyses performed are as follows.

The method for evaluating the quality of the crisps is based on the sum of the scores obtained regarding the various appearance and texture criteria, according to the frame of reference detailed below.

A first note on the general appearance of the crisps is obtained by summing:
a note from 1 to 4 regarding the shape of the crisps, with the note 1 for a very irregular shape and 4 for a nicely rounded shape.
A note from 1 to 2 regarding the color, with 1 for an unsatisfactory color (too dark/irregular) and 2 for an acceptable color.

A second note regarding the evaluation of the texture and of the level of expansion by summing:
A note from 1 to 5 regarding the hardness, with the note 1 for the “very hard” products/3 for the “crunchy” products and 5 for the “crisp” products.
A measurement of the level of radial expansion established by the ratio of the mean diameter of the crisps to the diameter of the die.

The table below summarizes the results obtained. The highest scores represent the best results.

| | Pea protein isolate | | |
|---|---|---|---|
| | No. 1 according to the | NUTRALYS ® | NUTRALYS ® |
| | invention | BF | S85F |
| Texture (/B) | 4.7 | 3.66 | 2.1 |
| Appearance | 4.5 | 1 | 1.5 |

The above results show that the best products were obtained with the pea protein isolate obtained according to the invention.

A sparingly functional and sparingly soluble protein such as the pea protein NUTRALYS® BF gives products that are average in terms of texture but unacceptable in terms of appearance. A soluble and functional protein such as NUTRALYS® S85F gives mediocre results in terms of appearance and texture.

### Example 23: High-Protein Nutritional Bars for Sportspeople

The technical challenge in high-protein nutritional bars is that of controlling the texture during the storage period of the product.

The reason for this is that high-protein nutritional bars have a tendency to harden over time.

Various hypotheses have been found in the literature to explain this phenomenon; especially the migration of water between the ingredients and protein aggregation.

The choice of protein(s) is thus crucial for the quality of the finished product.

Preparation of the nutritional bars according to various recipes:

| | | Recipes: 50% whey protein | | | | |
|---|---|---|---|---|---|---|
| | Ingredients | concentrate + 50% pea protein | | | | |
| | Ratio | 0%/ | 12.5%/ | 25%/ | 37.5%/ | 50%/ |
| | Pea protein isolate No. 2 | 50% | 37.5% | 25% | 12.5% | 0% |
| | according to the | | | | | |
| | invention | | | | | |
| | 7 NUTRALYS ® S85XF | | | | | |
| Syrup | Glucose/fructose syrup | 27% | 27% | 27% | 27% | 27% |
| | 7081 | | | | | |
| | NUTRIOSE ® | 11% | 11% | 11% | 11% | 11% |
| | Sorbitol syrup | 3.9% | 3.9% | 3.9% | 3.9% | 3.9% |
| | Sunflower oil | 2% | 2% | 2% | 2% | 2% |
| Powders | Whey protein | 20% | 20% | 20% | 20% | 20% |
| | concentrate 80 | | | | | |
| | NUTRALYS ® S85 XF | 20% | 15% | 10% | 5% | 0% |
| | Pea protein isolate No. 2 | 0% | 5% | 10% | 15% | 20% |
| | according to the | | | | | |
| | invention | | | | | |
| | Flavorings, amino | 1% | 1% | 1% | 1% | 1% |
| | acid | | | | | |
| | Defatted coconut powder | 1% | 1% | 1% | 1% | 1% |
| Topping | Dark chocolate powder | 15% | 15% | 15% | 15% | 15% |
| | TOTAL | 100% | 100% | 100% | 100% | 100% |

The amounts being given as weight percentages.

Analyses:

Measurement of the Hardness of the Nutritional Bars

Monitoring of the hardness (determined on an INSTRON® penetrometer—force required for the penetration of a knife into 40% of the thickness of the bar at constant speed) is performed over 1 month, with measurements at D₊₁, D₊₇, D₊₁₄, D₊₂₁, D₊₂₈ on the various recipes presented above.

The results are as follows:

Hardness of the bars (Instron measurements)
in newtons as a function of the pea protein ratios
and of the storage time.

| Ratio | | | | | |
|---|---|---|---|---|---|
| Pea protein isolate No. 2 according to | | | | | |
| the invention/NUTRALYS ® S85XF | D₊₁ | D₊₇ | D₊₁₄ | D₊₂₁ | D₊₂₈ |
| 0%/50% | 76.6 | 82.2 | 99.8 | 112 | 120.1 |
| 12.5%/37.6% | 54.4 | 64.9 | 72.4 | 90.4 | 95.6 |
| 25%/25% | 44 | 51.3 | 68.5 | 70 | 79.7 |
| 37.6%/12.5% | 31.1 | 31 | 54.8 | 61.8 | 70.3 |
| 50%/0% | 32.6 | 50.9 | 57.5 | 66.5 | 77.1 |

Increasing the degree of incorporation of the pea protein isolate No. 2 according to the invention is inversely proportional to the decrease in hardness of the bars, irrespective of the storage time.

An optimum is obtained for a ratio of 37.5% pea protein isolate No. 2 according to the invention/12.5% NUTRALYS® S85XF/50% WPC.

### Example 24. Mozzarella-Type Vegan Cheeses Containing Pea Protein Isolates

The vegan cheese recipe containing pea protein isolates No. 2 according to the invention is given in the following table:

The control is a recipe containing pea protein of NUTRALYS F85F type.

| | | No. 2 |
|---|---|---|
| | No. 1 | Pea protein isolate No. |
| | NUTRALYS ® | 2 according to the |
| Recipe | F85F | invention |
| Rapeseed oil | 7.98 | 7.98 |
| Coconut oil | 14.82 | 14.82 |
| Acetylated potato starch | 22.9 | 22.9 |
| CLEARAM PG 9020 | | |
| from Roquette Freres | | |
| pea protein or pea protein isolate | 5 | 5 |
| Citric acid | 0.3 | 0.3 |
| Salt | 1.7 | 1.7 |
| inactivated yeasts | 1.1 | 1.1 |
| Water | 41.7 | 41.9 |
| Cheese flavoring | 0.25 | 0.25 |
| Cassava starch | 4 | 4 |
| OSI Pea masker 9767A | 0.25 | 0.25 |
| Total | 100 | 100 |

The amounts being given as weight percentages.

The process for preparing the recipe is as follows:
add the water to a container equipped with a healing jacket (such as a Stephan Bowl—www.stephan-machinery.com/index.php?id=3) and heat to 50° C.,
add all the powder ingredients, except for the citric acid,
mix at 750 rpm for 2 minutes at 50° C.,
add the oils and mix for 2 minutes at 750 rpm,
add the citric acid and mix for 1 minute at 750 rpm,
heat the mixture to 75° C. while mixing regularly by hand so as to prevent it from browning,
stop the entry of steam into the jacket,
cook for 5 minutes, while mixing regularly,
stop the cooking and store at +6° C.

Analyses of color, texture, “shreddability”, and stability to freezing thawing and melting were undertaken.

While the color and texture of the two recipes are equivalent, the recipe with the pea protein isolate No. 2 has better “shreddability” behavior and better stability on melting. The taste is moreover acknowledged as being better with rectos No. 2.

1. A nutritional formulation containing a pea protein isolate, characterized in that the pea protein isolate:
contains between 0.5 and 2% of free amino acids,
has a viscosity at 20° C.:
from 11 to 18×10⁻³ Pa·s, at a shear rate of 10 s⁻¹,
from 9 to 16×10⁻³ Pa·s. et a shear rate of 40 s⁻¹, and
from 8 to 16×10⁻³ Pa·s. at a shear rate of 600 s⁻¹,
has a solubility:
from 30 to 40% in pH zones from 4 to 5
from 40 to 70% in pH zones from 6 to 8.

2. The formulation as claimed in claim 1, characterized in that the pea protein isolate has a digestibility expressed according to the Coefficient of Digestive Use (CDU) of between 93.5 and 95%.

3. The formulation as claimed in either of claims 1 and 2, characterized in that the pea protein isolate has a degree of hydrolysis (DH) of between 5 and 10%.

4. The formulation as claimed in any one of claims 1 to 3, characterized in that the pea protein isolate is presented, according to the SYMPHID test, as a protein of “rapid viscosity”, reflecting rapid duodenal assimilation of the constituent amino acids of said isolate.

5. The formulation as claimed in any one of claims 1 to 4, in which the pea protein isolate has been pasteurized at high temperature for a short time before being dried by atomization.

6. The formulation as claimed in any one of claims 1 to 5, in which the pea protein isolate represents 0.1-10%, 10-20%, 20-30%, 40-50%, 50-60%, 60-70%, 70-80%, 80-90% or 90-100% by weight of the total protein in the nutritional formulation.

7. The formulation as claimed in any one of claims 1 to 6, also comprising at least one milk protein.

8. The nutritional formulation as claimed in claim 7, in which the formulation is in powder form and comprises at least one pea protein isolate and at least one milk protein, in which the milk protein represents at least 10, 15, 20, 25, 30, 40, 45 or 50% by weight relative to the total weight of protein in the nutritional formulation in powder form.

9. A process for preparing a nutritional formulation in powder form comprising a pea protein isolate as claimed in any one of claims 1-8, comprising the steps of:
preparing a base powder comprising at least carbohydrates, vitamins and minerals;
dry-mixing, into the base powder, a pea protein isolate.

10. The process as claimed in claim 9, in which the base powder comprises milk protein, preferably at least 10, 15, 20, 25, 30, 40, 45 or 50% by weight relative to the total weight of protein in the nutritional formulation in powder form.

11. The process as claimed in claim 10, in which the pes protein isolate is dried by atomization and pasteurized at high temperature and for a short time before being dry-mixed into the base powder.

12. The process as claimed in claim 11, in which the pea protein isolate is pasteurized at high temperature and for a short time at a temperature ranging from 130° C. to 150° C. for a time from about 1 second to about 30 seconds.

13. The formulation as claimed in any one of claims 1-8, for use as a single protein source or as a food supplement, intended for infants; children and/or adults.

14. The formulation as claimed in claim 13, the formulation being:
beverage in powder form to be reconstituted or a ready-to-drink beverage,
a beverage for dietetic nutrition (sports, slimming) or for clinical nutrition, or
a dessert cream in clinical nutrition,
a nutritional liquid administered enterally,
a fermented milk of yoghurt type (stirred, Greek, drinking, etc.),
a dairy/plant-based beverage,
a dairy/plant-based cream (such as “coffee whitener”), iced dessert or sorbet,
a biscuit, muffin, griddle cake or nutritional bar,
a protein-enriched bread or gluten-free bread,
high-protein cereals, obtained by extrusion cooking.

15. The formulation as claimed in claim 14, in which the pea protein isolate represents:
between 40 and 100%, preferably between 50 and 100%, of the total protein in the nutritional formulation in powder form,
between 0.1% and 100% of the total protein for the ready-to-drink beverages for clinical nutrition and slimming, and
between 52% and 100% of the total protein for the ready-to-drink beverages for sports nutrition,
between 0.1% and 100% of the total protein for the fermented milks of yoghurt type,
between 0.1% and 100% of the total protein for the dairy beverages,
between 0.1% and 100% of the total protein for the dairy creams, load desserts or sorbets, more particularly between 50 and 100% of the total protein for the coffee whiteners,
between 5% and 100% of the total protein for the biscuits, muffins, griddle cakes and nutritional bars,
between 5% and 100% of the total protein for the protein enriched breads or gluten-trop breads,
between 5% and 100% of the total protein for the high-pro in cereals, obtained by extrusion cooking.

16. The use of the nutritional formulation as claimed in any one of claims 1-8, as a single protein source or as a food supplement, intended for infants, children and/or adults.
